(19) United States
(12) Patent Application Publication
(10) Pub. No.: US 2024/0424035 A1
(43) Pub. Date: Dec. 26, 2024

(54) BACTERIA PRODUCING GAMMA-AMINOBUTYRIC ACID (GABA) AND USES OF THE BACTERIA

(71) Applicants: Washington State University, Pullman, WA (US); Eastern Washington University, Cheney, WA (US)

(72) Inventors: Jean-Baptiste O. Roullet, Pullman, WA (US); Javier Ochoa-Reparaz, Cheney, WA (US); Kenneth Michael Gibson, Pullman, WA (US); Andrea Castillo, Cheney, WA (US)

(73) Assignees: Washington State University, Pullman, WA (US); Eastern Washington University, Cheney, WA (US)

(21) Appl. No.: 18/262,894

(22) PCT Filed: Jan. 26, 2022
(86) PCT No.: PCT/US2022/013881

§ 371 Date: Jul. 25, 2023

Related U.S. Application Data
(60) Provisional application No. 63/141,837, filed on Jan. 26, 2021.

(51) Int. Cl. A61K 35/744; A61P 1/00; A61P 37/02; C07K 14/245; C12N 9/88; C12N 15/74; C12P 13/00

(52) U.S. Cl. CPC A61K 35/744; A61P 1/00; A61P 37/02; C07K 14/245; C12N 9/88; C12N 15/746; C12P 13/005; C12Y 401/01015

(57) ABSTRACT

Probiotic bacteria are genetically modified to produce and excrete enhanced levels of the inhibitory neurotransmitter gamma-aminobutyric acid (GABA). Exemplary probiotic bacteria include Lactococcus lactis (L. lactis). The genetically modified probiotic bacteria can be prepared as an aqueous suspension and/or incorporated into food and nutraceuticals for oral administration to a subject. An oral formulation including the genetically modified probiotic bacteria can be used to treat inflammatory diseases and/or behavioral disorders caused by or associated with GABA-GABA signaling deficiency or by excess of excitatory neurotransmitters (such as glutamate). Inflammatory conditions that can be treated with the genetically modified probiotic bacteria include multiple sclerosis (MS) and irritable bowel disease (IBD).

### CROSS-REFERENCE TO RELATED APPLICATION

This application claims priority to U.S. Provisional Application No. 63/141,837, filed on Jan. 26, 2021, which is incorporated by reference herein in its entirety.

### STATEMENT REGARDING FEDERALLY SPONSORED RESEARCH OR DEVELOPMENT

This invention was made with government support under grant number NS107743 awarded by National Institutes of Health. The government has certain rights in the invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 2MR3308_ST25.txt. The text file is 32 KB, was created on Jan. 24, 2022, and is being submitted electronically via EFS-Web.

### FIELD OF THE DISCLOSURE

The present disclosure relates to probiotic bacteria engineered to enhance production of gamma-aminobutyric acid (GABA) and uses of these bacteria including methods of treating inflammatory diseases and/or behavioral disorders.

### BACKGROUND OF THE DISCLOSURE

Gamma-aminobutyric acid (GABA) is an amino acid chemical messenger in the brain that inhibits nerve transmission, thus slowing down brain activities. Low levels of GABA, known as an inhibitory neurotransmitter, may be responsible for stress disorders, anxiety disorders, and/or sleep disorders (e.g., insomnia). GABA is used as a supplement to reduce insomnia, reduce depression, enhance immunity, relieve anxiety, control hypertension, fight obesity, and improve visual cortical function.

GABA has a role in regulating the immune system, as receptors that bind GABA are found on the surface of immune cells. When GABA binds to these receptors, the receptors are activated and change the behavior of the immune cells. Thus, GABA may influence immune system function in an organism.

Low GABA levels or impaired GABA receptor mediated signaling are associated with numerous disorders, including anxiety, autism spectrum disorders, schizophrenia, Huntington's disease, epilepsy, and multiple sclerosis.

GABA is naturally present in foods such as tea, tomato, soybean, rice, and spinach, and in fermented foods such as kimchi, miso, and tempeh, but not at levels high enough to be therapeutic.

Subjects with neurological diseases characterized by low GABA levels and/or impaired GABA receptor mediated signaling or subjects having inflammatory diseases may benefit from administration of GABA.

### SUMMARY OF THE DISCLOSURE

The present disclosure provides for probiotic bacteria that have been genetically modified to produce and excrete enhanced levels of the inhibitory neurotransmitter gamma-aminobutyric acid (GABA), methods of making the genetically modified probiotic bacteria, and oral compositions including the genetically modified probiotic bacteria.

In embodiments, the probiotic bacteria include lactic acid bacteria. In embodiments, the lactic acid bacteria belong to a genus selected from the group consisting of Bifidobacterium, Carnobacterium, Enterococcus, Lactobacillus, Lactococcus, Leuconostoc, Oenococcus, Pediococcus, Propionibacterium, Streptococcus, Tetragenococcus, Vagococcus, and Weissella. In embodiments, the probiotic bacteria include Lactococcus lactis (L. lactis).

Embodiments provide for a genetically modified probiotic bacterium including a heterologous gene encoding a glutamic acid decarboxylase (EC 4.1.1.15) and a heterologous gene encoding a glutamate/GABA antiporter. In embodiments, the heterologous gene encoding the glutamic acid decarboxylase includes gadB. In embodiments, the gadB includes Lactococcus lactis gadB having at least 80% sequence identity to a sequence as set forth in SEQ ID NO: 1. In embodiments, the gadB includes Lactococcus lactis gadB having an amino acid sequence as set forth in SEQ ID NO: 1. In embodiments, the gadB includes Lactococcus lactis gadB having at least 80% sequence identity to a nucleic acid sequence encoding a sequence as set forth in SEQ ID NO: 1. In embodiments, the gadB includes Lactococcus lactis gadB having at least 80% sequence identity to a nucleic acid sequence as set forth in SEQ ID NO: 3. In embodiments, the gadB includes Lactococcus lactis gadB having a sequence as set forth in SEQ ID NO: 3.

In embodiments, the heterologous gene encoding the glutamate/GABA antiporter includes gadC. In embodiments, the gadC includes Lactococcus lactis gadC having at least 80% sequence identity to a sequence as set forth in SEQ ID NO: 2. In embodiments, the gadC includes Lactococcus lactis gadC having an amino acid sequence as set forth in SEQ ID NO: 2. In embodiments, the gadC includes Lactococcus lactis gadC having at least 80% sequence identity to a nucleic acid sequence encoding a sequence as set forth in SEQ ID NO: 2. In embodiments, the gadC includes Lactococcus lactis gadC having at least 80% sequence identity to a nucleic acid sequence as set forth in SEQ ID NO: 4. In embodiments, the gadC includes Lactococcus lactis gadC having a sequence as set forth in SEQ ID NO: 4.

In embodiments, both the heterologous gene encoding a glutamic acid decarboxylase and the heterologous gene encoding a glutamate/GABA antiporter are operably linked to a heterologous promoter. In embodiments, the heterologous promoter includes an inducible promoter. In embodiments, the inducible promoter includes PgroES, pL, pR, cspA, pLac, pBad, pTac, Ptrp, PhoA, recA, proU, sct, tetA, cadA, cadR, nar, p170, nisin-inducible promoter, or P_{aguB}. In embodiments, the heterologous promoter includes a constitutive promoter. In embodiments, the constitutive promoter includes P1, P2, P3, P4, P5, P6, P7, P8, P32, P45, LacA, PPepN, P6C, P13C, or PTS-IIC. In embodiments, the constitutive promoter includes a P2 promoter. In embodiments, the P2 promoter has a sequence as set forth in SEQ ID NO: 12. In embodiments, the constitutive promoter includes a P5 promoter. In embodiments, the P5 promoter has a sequence as set forth in SEQ ID NO: 13. In embodiments, the constitutive promoter includes a P8 promoter. In embodiments, the P8 promoter has a sequence as set forth in SEQ ID NO: 5 or SEQ ID NO: 6. In embodiments, the P8 promoter includes a short version of the promoter as set forth in SEQ ID NO: 5.

In embodiments, the genetically modified probiotic bacterium includes endogenous glutamic acid decarboxylase and glutamate/GABA antiporter genes. In embodiments, the heterologous gene encoding the glutamic acid decarboxylase and the heterologous gene encoding the glutamate/GABA antiporter is part of an expression cassette of a genetic construct. In embodiments, the genetic construct is not integrated in the genome of the genetically modified probiotic bacterium. In embodiments, the genetic construct is integrated into the genome of the genetically modified probiotic bacterium. In embodiments, the integrated genetic construct disrupts an endogenous gene. In embodiments, the endogenous gene is leuA.

In embodiments, the genetic construct further includes an upstream homology arm and a downstream homology arm. In embodiments, the upstream and downstream homology arms include sequences homologous to an endogenous gene of a probiotic bacterium. In embodiments, the probiotic bacterium is L. lactis and the endogenous gene is leuA. In embodiments, the upstream homology arm has a sequence having at least 90% sequence identity to the sequence as set forth in SEQ ID NO: 9. In embodiments, the upstream homology arm has a sequence as set forth in SEQ ID NO: 9. In embodiments, the downstream homology arm has a sequence having at least 90% sequence identity to the sequence as set forth in SEQ ID NO: 10.

In embodiments, the downstream homology arm has a sequence as set forth in SEQ ID NO: 10. In embodiments, the genetic construct includes a sequence as set forth in SEQ ID NO: 11. In embodiments, the genetic construct further includes a selectable marker. In embodiments, the selectable marker confers antibiotic resistance, provides an essential gene, confers chemical resistance, and/or includes a visual marker. In embodiments, the antibiotic resistance is erythromycin resistance.

Embodiments provide for genetically modified probiotic bacteria that produce an amount of gamma-aminobutyric acid (GABA) that is 2-fold to 200-fold greater as compared to an amount of GABA produced by a control. In embodiments, the control is probiotic bacteria of the same genus or species that has not been genetically modified. In embodiments, the control is probiotic bacteria of the same genus or species that has been genetically modified with a control plasmid. In embodiments, the genetically modified probiotic bacteria produce 500 ng/mL bacteria to 60,000 ng/ml bacteria.

Embodiments provide for a genetic construct including: a promoter operably linked to: a gene encoding a glutamic acid decarboxylase (EC 4.1.1.15); and a gene encoding a glutamate/GABA antiporter. In embodiments, the genetic construct includes 5′ to 3′: the promoter, the gene encoding a glutamate/GABA antiporter, and the gene encoding a glutamic acid decarboxylase. Embodiments provide for a method of preparing genetically modified probiotic bacteria, including introducing the genetic construct into probiotic bacteria to obtain genetically modified probiotic bacteria; and culturing the genetically modified probiotic bacteria in media. In embodiments, the culturing includes growing the genetically modified probiotic bacteria at 20° C. to 50° C. In embodiments, the culturing further includes adding glutamic acid HCl to the media. In embodiments, the culturing includes growing the genetically modified probiotic bacteria to an OD600 of 0.5 to 2. Embodiments provide for a method of producing gamma-aminobutyric acid (GABA), including culturing the genetically modified probiotic bacteria in media.

Embodiments provide for a composition including the genetically modified probiotic bacteria and a pharmaceutically acceptable carrier. In embodiments, the composition includes an oral composition. In embodiments, the composition includes a solid or a liquid. In embodiments, the solid includes a lyophilized powder. In embodiments, the composition includes a liquid suspension. In embodiments, the oral composition is part of a dairy product. In embodiments, the dairy product includes yogurt, milk, cheese, kefir, ice cream, or butter. In embodiments, the composition further includes a prebiotic. In embodiments, the prebiotic includes fiber, amino acids, oligosaccharides, and polyamines.

An oral composition including the genetically modified probiotic bacteria can be administered to a subject to treat a disease or disorder. In embodiments, the disease or disorder is associated with GABA deficiency, impaired GABA receptor mediated signaling, and/or an excess of excitatory neurotransmitters. In embodiments, the disease or disorder associated with GABA deficiency, impaired GABA receptor mediated signaling, and/or an excess of excitatory neurotransmitters includes alcoholism, depression, anxiety, autism, multiple sclerosis, schizophrenia, Parkinson's Disease, Huntington's disease, epilepsy, post-traumatic stress disorder (PTSD), and stroke and its complications. In embodiments, the disease or disorder is associated with inflammation. In embodiments, the disease or disorder is an inflammatory autoimmune disease. In embodiments, the inflammatory autoimmune disease includes cachexia, inflammatory bowel diseases (IBD), psoriatic arthritis, rheumatoid arthritis, Type 1 diabetes, Type 2 Diabetes, Sjögren's syndrome, systemic lupus erythematosus, celiac disease, Graves' disease, Hashimoto's thyroiditis, Addison's disease, dermatomyositis, psoriasis, chronic inflammatory demyelinating polyneuropathy, Guillain-Barre syndrome, myasthenia gravis, and vasculitis. In embodiments, the IBD is Crohn's disease or ulcerative colitis.

### BRIEF DESCRIPTION OF THE DRAWINGS

Some of the drawings submitted herein may be better understood in color. Applicants consider the color versions of the drawings as part of the original submission and reserve the right to present color images of the drawings in later proceedings.

FIGS. 1A, 1B. FIG. 1A. (i) The plasmid pGh9: ISS1, containing a temperature sensitive (Ts) origin of replication and the erm gene that confers erythromycin resistance, was used for initial cloning and characterization of Px-GAD constructs. (ii) The Px-GAD constructs (P2-GAD, P5-GAD, and P8-GAD) were synthesized to include the L. lactis gadC gadB gene operon regulated by the non-native constitutive promoters and ribosome binding sites of P2, P5 or P8s (Zhu et al. FEMS Microbiol Lett 2015, 362). The Px-GAD constructs were synthesized with regions of the L. lactis leuA gene upstream and downstream for future integration experiments. Pstl cloning sites were included at the 5′ and 3′ ends of leuA to facilitate cloning into the Pstl cloning site of pGh9: ISS1. (iii) pPx-GAD with Px-GAD cloned into the Pstl site of pGh9: ISS1. For the integration of Px-GAD into the L. lactis chromosome, Px-GAD was cloned into plasmid pBVGh, a modified version of pGh9: ISS1 that allows for targeted integration. FIG. 1B. Schematic for integration of pBVGh-Px-GAD into L. lactis. pBVGh-leuA: Px-gadCB was used to transform L. lactis and selected by erythromycin resistance (30° C.). Integration of pBVGh-Px-GAD into the leuA locus of L. lactis and subsequent removal of the pBVGh backbone was facilitated by incubating cells at 37° C. and 30° C. without plasmid selection (media lacks erythromycin) (Blancato and Magni. Lett. Appl. Microbiol. 2010, 50, 542-546).

FIG. 2. Genetically engineered L. lactis having one more copy of the gadB gene and one more copy of the gadC gene (GAD-L. lactis) produce enhanced levels of GABA. GABA levels were quantified in the supernatants of L. lactis cultures obtained at increasing absorbance (OD600 of 0.5, 1, 1.5, and 2): wild type (WT), L. lactis strain IL403; pGh9: ISS1, L. lactis with pGh9: ISS1 plasmid vector; P5, genetically engineered L. lactis having a construct with promoter P5 operably linked to gadB and gadC genes; P8s, genetically engineered L. lactis having a construct with promoter P8 short operably linked to gadB and gadC genes. Mean average of six assay replicates GABA levels compared by mixed-effects analysis followed by Tukey's multiple comparisons test.

FIG. 3. Genetically engineered GAD-L. lactis producing enhanced GABA levels protect against experimental autoimmune encephalomyelitis (EAE) in mice. EAE was induced on day 0 and clinical scores monitored daily. Oral treatments with bacteria were administered by gavages (5 days per week, at 5×10⁸ CFU/mouse, resuspended in M17 medium). Sham controls received sterile M17 medium. pGh9: ISS1, L. lactis with pGh9: ISS1 plasmid vector; P8s, genetically engineered L. lactis having a construct with promoter P8 short operably linked to gadB and gadC genes. n=10 mice per group. Medium Group: M17 medium only (0.1 mL, 5 days/week). pGh9: ISS1 Group: L. lactis with empty pGh9: ISS1 vector (5×10⁸ CFU/mouse in 0.1 mL, 5 days/week). P8 Group: GAD-L. lactis with P8 short promoter operably linked to gadB and gadC genes (5×10⁸ CFU/mouse in 0.1 mL, 5 days/week). Mean average of EAE clinical scores compared by repeated measures of ANOVA, followed by Tukey's multiple comparisons test. *** p<0.001.

FIG. 4. GAD-L. lactis producing enhanced GABA levels reduce the severity of EAE in mice. EAE was induced on day 0 and clinical scores monitored daily. Oral treatments with bacteria containing constructs were administered by gavages (5 days per week, at 5×10⁸ CFU/mouse, resuspended in M17 medium). Sham controls received sterile M17 medium. pGh9: ISS1, L. lactis with pGh9: ISS1 plasmid vector; P8s, genetically engineered L. lactis having a construct with promoter P8 short operably linked to gadB and gadC genes. n=10 mice per group. Distribution of EAE scores at days 19 and 25 after EAE induction. 0 is a healthy animal with no disease; 0.5, a distal limp tail; 1, completely limp tail or isolated weakness of gait without a limp tail; 1.5, a limp tail and hind limb weakness; 2, unilateral partial hind limb paralysis; 2.5, bilateral partial hind limb paralysis; 3, complete bilateral hind limb or partial hind and front limb paralysis; 3.5, complete bilateral hind limb paralysis and partial front limb paralysis. 5, moribund or dead animal.

FIG. 5. GAD L. lactis construct producing enhanced GABA levels prevent body loss during EAE. EAE was induced on day 0 and clinical scores monitored daily. Oral treatments with bacterial constructs were administered by gavages (5 days per week, at 5×10⁸ CFU/mouse, resuspended in M17 medium. Sham controls received sterile M17 medium. pGh9: ISS1, L. lactis with pGh9: ISS1 plasmid vector; P8s, L. lactis construct with promoter P8 short and GAD. n=10 mice per group. Mean average of % body weights versus initial weights, compared by repeated measures of ANOVA, followed by Tukey's multiple comparisons test. *, p<0.05; **, p<0.01.

FIG. 6. Expression of gadB conferred by constitutive expression. Total RNA was isolated from L lactis strains (L. lactis unmodified (WT), pGh9: ISS1-L. lactis (P), pGh-P2-GAD-L. lactis (P2), pGh-P5-GAD-L. lactis (P5) and pGh-P8s-GAD-L. lactis (P8s). Reverse transcription and qPCR were carried out using the ReverTra Ace™ qPCR RT Master Mix with gDNA Remover (Toyobo) and THUNDERBIRD™ Next SYBR™ qPCR Mix (Toyobo), respectively. Relative expression of gadB was determined using 16s rRNA as a reference gene. The ΔΔCt was calculated by comparing the ΔCt of L. lactis with P, P2, P5 or P8s to the ΔCt of the WT strain. The fold-change in expression was calculated as 2-44Ct. An ANOVA (P=0.031) and Dunnett's multiple comparison test were used to compare means. gadB expression is significantly higher in P8 than in the WT strain (P=0.0325).

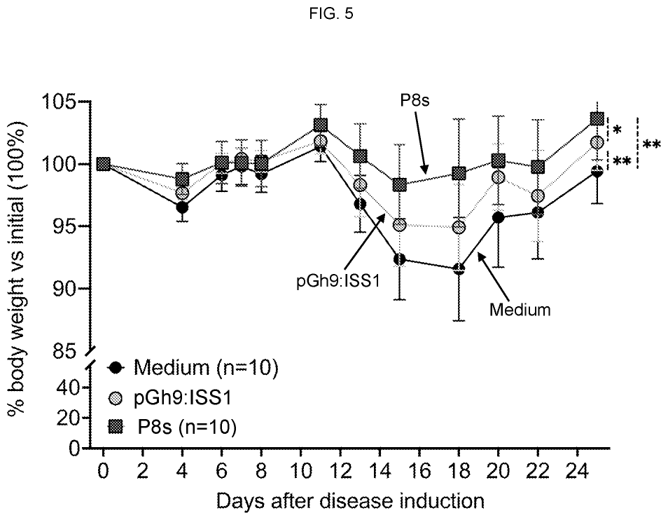

FIG. 7. GABA produced by L. lactis strains. A GABA ELISA (LDN®, Nordhorn, Germany) was used to measure GABA levels in L. lactis strain supernatants (pGh9: ISS1-L. lactis (P) and pGh-P8s-GAD-L. lactis (P8). Strains were cultured in GM17+erm alone (0) or with glutamic acid HCl (50 mM (1), 150 mM (2) or 200 mM (3)) and incubated at 30° C. for 3 hours or the indicated times. GABA concentration was expressed as GABA concentration of the sample minus the GABA concentration of the media control, normalized for CFU/mL. All P8 strains cultured with glutamic acid-HCl produced significantly more GABA than the P8 strain cultured in M17+erm (1, P=0.002; 2 and 3, P<0.001) or P strains cultured with or without glutamic acid-HCl (P_0 to P8_1, P=0.0017; P8_2 and 3, P<0.001; P_1 to P8_1, P=0.0042, P_1 to P8_2 and 3, P<0.001; P_2 to P8_1, P=0.0037, P_2 to P8_2 and 3, P<0.001; P_3 to P8_1, P=0.0038, P_3 to P8_2 and 3, P<0.001). The ANOVA was P<0.001 and the group means were analyzed by Tukey's multiple comparison. GABA production increased over time in P8 cultured in 200 mM glutamic acid-HCl. The table shows the strains analyzed and the GABA concentration at T=3 hrs. The GABA is presented as ng/ml, and adjusted for variations in CFU/mL to 10⁸ CFU/mL.

FIG. 8. GAD L. lactis construct producing enhanced GABA levels improved survival in a mouse model of irritable bowel disease (IBD). The 2,4,6-trinitrobenzenesulfonic acid (TNBS) model of IBD in BALB/c mice was used to compare survival rates and colon length in mice treated orally with P8s-GAD-L. lactis, L. lactis (pGh9: ISS1), and sterile water. Colitis was induced by administration of TNBS via rectum. Ten (10) female BALB/c mice at 8 weeks old were randomly divided into four groups: Group 1, Colitis group control (n=10), drinking normal water for two weeks before and after colitis induction; Group 2, The L. lactis (P) group (n=10), drinking L. lactis (pGh9: ISS1) drinking water for two weeks before and after the colitis induction. Group 3, The P8-GAD-L. lactis group (n=10) received P8s-GAD-L. lactis in their drinking water for two weeks before and after colitis induction. Group 4, Healthy control group (n=5). Disease was induced in two stages. On day-7, mice of groups 1, 2, and 3 were pre-sensitized by shaving a 1.5×1.5 cm square of skin on the back of the mouse and applying 150 μl of 5% TNBS emulsion (4 volumes of acetone/olive oil) to the shaved skin. On day 0, 100 μl of 5% TNBS solution (weight/volume) in autoclaved water and 1 volume of absolute ethanol was administered into the rectum using a 3.5 F catheter connected to a 1 ml syringe. Disease was monitored for five days, and survival rates and colon length determined.

FIG. 9. GAD L. lactis construct producing enhanced GABA levels improved colon length retention. The TNBS model of IBD as described in FIG. 8 was used.

### DETAILED DESCRIPTION

The inhibitory neurotransmitter gamma-aminobutyric acid (GABA) is an essential regulator of the central nervous system (Mazzoli and Pessione. Frontiers in Microbiology 2016, 7, 1934). GABA also regulates the function of the immune system by interacting with GABA-specific receptors expressed on immune cells (Bhat et al. Proceedings of the National Academy of Sciences of the United States of America 2010, 107, 2580-2585; Tian et al. Journal of Neuroimmunology 1999, 96, 21-28; Tian et al. The Journal of Immunology 2004, 173, 5298-5304; Tian et al. Autoimmunity 2011, 44, 465-470). As a result, GABAergic dysfunction could negatively affect immune-related diseases (such as multiple sclerosis (MS) and inflammatory bowel disease (IBD)), as well as behavioral disorders. Reduced serum and intestinal levels of GABA are found in multiple sclerosis (MS) patients (De Stefano and Giorgio. Brain 2015, 138, 2467-2468; Cao et al. European radiology 2018, 28, 1140-1148; Yalçinkaya et al. Multiple Sclerosis and Related Disorders 2016, 9, 60-61). Because of their role in modulating GABA/glutamate levels, intestinal microbes can be targeted for GABAergic, inhibitory, and immunomodulatory effects.

Intestinal barrier disruption has been associated with multiple immune-related diseases, such as irritable bowel disease (IBD) and MS. Some of those inflammatory mediators, such as tumor necrosis factor alpha (TNF-α) production is enhanced in the gut and brains of mice suffering from MS-like disease. Lactic acid bacteria (LABs) serve as principal agents of multiple probiotics and are currently being evaluated as efficient vectors for delivering therapeutics (Colombo et al. BMC Microbiol 2018, 18, 219). LABs impact immunological responses by either inducing or inhibiting responses and serve as normal resident species of the intestinal microbiome. Experimentally, probiotics based on L. lactis have been shown to reduce inflammatory mediators that serve as promoters of intestinal barrier permeability (Song et al. Microbial cell factories 2017, 16, 55). In embodiments, supplementing intestinal GABA, by introducing an engineered probiotic L. lactis designed to express additional copies of glutamic acid decarboxylase enzyme (GAD) and a GABA/glutamate antiporter (GadC) to produce high levels of GABA, can affect the progression of disease by reducing inflammation and intestinal permeability and affecting the structure of the microbiota. L. lactis genetically modified to produce GABA thus provides a new probiotic-based treatment strategy with enhanced protective effects.

In embodiments, increased intestinal and systemic GABA levels induced by treatment with a probiotic designed to enhance synthesis of GABA can decrease inflammation by promoting immunomodulatory T cell responses. In the current understanding of the pathogenesis of MS, intestinal dysbiosis contributes to CNS GABA deficiency and disease exacerbation. In embodiments, treating intestinal dysbiosis with a probiotic capable of delivering supplemental GABA to the host would provide significant clinical benefits.

Past studies show that the production of gamma-aminobutyric acid (GABA), an inhibitory neurotransmitter, by gut bacteria is decreased in patients with multiple sclerosis (MS). In EAE mice, the amount of intestinal GABA producers decreases in the early stages of the disease (Colpitts et al. Gut Microbes, 2017, 8, 561-573). Furthermore, the administration of GABAergic compounds ameliorates the progression of experimental autoimmune encephalomyelitis (EAE), a murine model of MS. The present disclosure describes that modifying the microbiota of mice with GABA-producing bacteria reduces the severity of EAE. Lactococcus lactis was genetically modified to include an extra copy of a gene encoding a glutamic acid decarboxylase enzyme (GAD), which synthesizes GABA, and an extra copy of a gene encoding the GABA/glutamate antiporter (GadC), both operably linked to a constitutive promoter. This modified L. lactis strain (GAD-L. lactis) produced significantly increased GABA, measured by ELISA, compared to a control L. lactis strain. In embodiments, the control L. lactis strain is an L. lactis strain that is not genetically modified or is an L. lactis strain with an empty control plasmid (P-L. lactis). These strains were compared with sham treatment as probiotic treatments against EAE. EAE-induced C57BL/6 mice were divided into three groups: sham group (EAE induced, treated with autoclaved media; n=10); EAE mice treated with P-L. lactis (n=10); and EAE mice treated with GAD-L. lactis (n=10). The treatments occurred five times a week via oral gavage (5×10⁸ CFU/mouse). The oral administration of GAD-L. lactis significantly reduced the severity of EAE and body weight loss during disease, compared to both the P-L. lactis and sham groups. The results indicate that oral treatment with a probiotic strain that produces enhanced GABA levels protects against the progression of CNS demyelination. In embodiments, immunophenotyping studies can elucidate the mechanism of action of GAD-L. lactis and its potential as a novel approach to treat autoimmune disorders.

In embodiments, use of a constitutive promoter for heterologous gene expression is better suited for in situ purposes where continuous expression at constant levels is desired. There is a relative paucity of constitutive promoters available for heterologous gene expression in Lactococcus lactis. Choosing the correct promoter to achieve therapeutic expression of a target gene in L. lactis remains a challenge and successful expression can be dependent upon the compound produced.

Aspects of the current disclosure are now described with additional details and options as follows: Gamma-aminobutyric acid (GABA) and its production by probiotic bacteria genetically modified to express heterologous genes that function in GABA production; Genetic modification of bacteria; Probiotic bacteria; Compositions; Methods of Use; Variants; Closing Paragraphs; Exemplary Embodiments; Experimental Examples; and References. These headings do not limit the interpretation of the disclosure and are provided for organizational purposes only.

Gamma-aminobutyric acid (GABA) and its production by probiotic bacteria genetically modified to express heterologous genes that function in GABA production. GABA, also known as 4-aminobutanoate, is the principal inhibitory neurotransmitter in the central nervous system (CNS) and has the following structure:

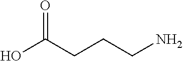

GABA is produced by neurons in the CNS and by intestinal bacteria such as Lactococcus lactis and Lactobacillus. It is also found in many fermented foods because lactic acid bacteria produce it. The bacteria produce GABA through the action of a glutamic acid decarboxylase (GAD) system, which includes a GAD enzyme encoded by gadA or gadB and a glutamate/GABA antiporter encoded by gadC.

Glutamate is transported into a cell through GadC antiporter. GAD enzyme decarboxylates glutamate to GABA, and the reaction can be depicted as follows:

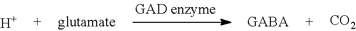

The decarboxylation of glutamate is catalyzed by GAD with cofactor pyridoxal-5′-phosphate (PLP), resulting in formation of GABA and CO₂ as byproduct. The produced GABA is exported outside a cell by GadC antiporter.

In embodiments, a GAD enzyme can be activated by cations including Ba²⁺, Ca²⁺, Co²⁺, Fe³⁺, Mg²⁺, Mn²⁺, Mo⁶⁺, Nat, NH⁴⁺, Zn²⁺. In embodiments, a GAD enzyme does not need cations for activation. In embodiments, a GAD enzyme functions in the cytoplasm of a cell. In embodiments, a GAD enzyme functions in a pH range of 4.0 to 8.0, or 6.0 to 8.0, or 6.5 to 7.5, or near neutral pH. In embodiments, a GAD enzyme functions at pH 4.0, pH 4.1, pH 4.2, pH 4.3, pH 4.4, pH 4.5, pH 4.6, pH 4.7, pH 4.8, pH 4.9, pH 5.0, pH 5.1, pH 5.2, pH 5.3, pH 5.4, pH 5.5, pH 5.6, pH 5.7, pH 5.8, pH 5.9, pH 6.0, pH 6.1, pH 6.2, pH 6.3, pH 6.4, pH 6.5, pH 6.6, pH 6.7, pH 6.8, pH 6.9, pH 7.0, pH 7.1, pH 7.2, pH 7.3, pH 7.4, pH 7.5, pH 7.6, pH 7.7, pH 7.8, pH 7.9, or pH 8.0. In embodiments, a GAD enzyme functions in a temperature range of 30° C. to 60° C., or 35° C. to 50° C., or 35° C. to 45° C.

In embodiments, a GAD enzyme expressed in a genetically modified probiotic bacterium of the disclosure includes L. lactis gadB Accession no. AAK05388.1 (SEQ ID NO: 1), L. lactis subsp. cremoris MG1363 (AAC46188.1), L. brevis CGMCC1306 (ADG02973.1), L. reuteri TD01 (AGR65020.1), and S. thermophilus Y2 (ABI31651.2). In embodiments, a GAD enzyme expressed in a genetically modified probiotic bacterium of the disclosure includes a GAD enzyme disclosed in US20190070225.

In embodiments, a GAD enzyme expressed in a genetically modified probiotic bacterium of the disclosure (e.g., L. lactis) includes an L. lactis GadB enzyme having an amino acid sequence as set forth in SEQ ID NO: 1.

In embodiments, a GAD enzyme expressed in a genetically modified probiotic bacterium of the disclosure (e.g., L. lactis) has a sequence having at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or at least 99.9% sequence identity to an L. lactis GadB amino acid sequence as set forth in SEQ ID NO: 1, provided the GAD enzyme retains functional activity. In embodiments, a GAD enzyme having functional activity can convert glutamate to GABA.

In embodiments, a GAD enzyme expressed in a genetically modified probiotic bacterium of the disclosure (e.g., L. lactis) is encoded by an L. lactis gadB gene having a nucleic acid sequence as set forth in SEQ ID NO: 3.

In embodiments, a gadB gene expressed in a genetically modified probiotic bacterium of the disclosure (e.g., L. lactis) has a sequence having at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or at least 99.9% sequence identity to a nucleic acid sequence encoding an L. lactis GadB enzyme as set forth in SEQ ID NO: 1, provided the gadB gene encodes a GAD enzyme that retains functional activity. In embodiments, a GAD enzyme having functional activity can convert glutamate to GABA.

In embodiments, a gadB gene expressed in a genetically modified probiotic bacterium of the disclosure (e.g., L. lactis) has a sequence having at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or at least 99.9% sequence identity to a nucleic acid sequence as set forth in SEQ ID NO: 3.

In embodiments, an L. lactis GadB enzyme having at least 80% sequence identity to an L. lactis GadB amino acid sequence as set forth in SEQ ID NO: 1 encodes a functional GAD enzyme. In embodiments, an L. lactis gadB nucleic acid sequence having at least 80% sequence identity to a nucleic acid encoding a sequence as set forth in SEQ ID NO: 1 encodes a functional GAD enzyme. In embodiments, an L. lactis gadB nucleic acid sequence having at least 80% sequence identity to a nucleic acid sequence as set forth in SEQ ID NO: 3 encodes a functional GAD enzyme. A functional GAD enzyme can convert glutamate to GABA.

In embodiments, a GadC glutamate/GABA antiporter expressed in a genetically modified probiotic bacterium of the disclosure includes L. lactis gadC Accession no. AAK05389.1 (SEQ ID NO: 2). In embodiments, a GadC glutamate/GABA antiporter expressed in a genetically modified probiotic bacterium of the disclosure includes an antiporter listed in Table 1.

TABLE 1
Accession numbers for GadC amino acid sequences
that can be used in the present disclosure.

| Genus/species | GenBank accession no. |
|---|---|
| Lactococcus lactis subsp. lactis CNCM I-1631 | EHE94112.1 |
| Lactococcus | WP_010905871.1 |
| Lactococcus lactis | WP_150891040.1 |
| Lactococcus | WP_023189201.1 |
| Lactococcus lactis | WP_081144310.1 |
| Lactococcus lactis | WP_058205725.1 |
| Lactococcus lactis | WP_085624903.1 |
| Lactococcus lactis | WP_032947764.1 |
| Lactococcus | WP_129881675.1 |
| Lactococcus cremoris | WP_153951670.1 |
| Lactococcus lactis | WP_038600763.1 |
| Lactococcus lactis | WP_033898929.1 |
| Lactococcus lactis subsp. lactis | QGA67144.1 |
| Lactococcus lactis | WP_017864551.1 |
| Lactococcus lactis | WP_058217062.1 |
| Lactococcus lactis | WP_095586285.1 |
| Lactococcus lactis | WP_058206447.1 |
| Lactococcus lactis | WP_058218915.1 |
| Lactococcus lactis | WP_058203111.1 |
| Lactococcus lactis | WP_143459851.1 |
| Lactococcus lactis | WP_143460981.1 |
| Lactococcus lactis subsp. lactis IO-1 | BAL51234.1 |
| Lactococcus lactis | WP_098393744.1 |
| Lactococcus lactis | WP_058223168.1 |
| Lactococcus lactis subsp. lactis | KRO22700.1 |
| Lactococcus lactis | WP_058212940.1 |
| Lactococcus lactis | WP_029343973.1 |
| Lactococcus lactis | WP_200984604.1 |
| Lactococcus lactis | HAF56738.1 |
| Lactococcus lactis | WP_168784534.1 |
| Lactococcus lactis | WP_195228743.1 |
| Lactococcus lactis | WP_103054789.1 |
| Lactococcus lactis | WP_058211961.1 |
| Lactococcus lactis | WP_146978054.1 |
| Lactococcus lactis | WP_201179739.1 |
| Lactococcus lactis | WP_195918821.1 |
| Lactococcus lactis | WP_042230406.1 |
| Lactococcus lactis | WP_058204664.1 |
| Lactococcus lactis | WP_212656429.1 |
| Lactococcus lactis | WP_060416597.1 |
| Lactococcus lactis | WP_012897892.1 |
| Lactococcus lactis | WP_153241637.1 |
| Lactococcus lactis | WP_098403370.1 |
| Lactococcus lactis | WP_057720633.1 |
| Lactococcus lactis | WP_031299517.1 |
| Lactococcus lactis subsp. lactis | KLK96690.1 |
| Lactococcus lactis | WP_195935290.1 |
| Lactococcus lactis | WP_129299998.1 |
| Lactococcus lactis | WP_191670602.1 |
| Lactococcus | WP_225513459.1 |
| Lactococcus cremoris | MRM56382.1 |
| Lactococcus cremoris | PCS20430.1 |
| Lactococcus | WP_014572691.1 |
| Lactococcus cremoris | WP_096815615.1 |
| Lactococcus cremoris | WP_043734430.1 |
| Lactococcus cremoris | WP_015082498.1 |
| Lactococcus | WP_063283126.1 |
| Lactococcus cremoris | WP_011676216.1 |
| Lactococcus lactis subsp. cremoris MG1363 | CAL97771.1 |
| Lactococcus cremoris | WP_021037314.1 |
| Lactococcus | WP_021165319.1 |
| Lactococcus cremoris | WP_046781322.1 |
| Lactococcus cremoris | WP_014735013.1 |
| Lactococcus lactis subsp. cremoris TIFN7 | EQC82582.1 |
| Lactococcus lactis subsp. cremoris TIFN6 | EQC54490.1 |
| Lactococcus cremoris | WP_217205714.1 |
| Lactococcus | WP_014570615.1 |
| Lactococcus cremoris | WP_228764149.1 |
| Lactococcus lactis | WP_133278196.1 |
| Lactococcus lactis subsp. lactis | GAM80333.1 |
| Lactococcus lactis subsp. lactis | PCS19230.1 |
| Enterococcus avium | OJG13825.1 |
| Enterococcus avium | WP_207486600.1 |
| bacterium CH2-D8-79 | NBK65171.1 |
| Enterococcus | WP_016179791.1 |
| Enterococcus avium | WP_102871747.1 |
| Enterococcus avium | WP_048720429.1 |
| Enterococcus avium | WP_123864246.1 |
| Lactococcus sp. NH2-7C | WP_225511441.1 |
| Enterococcus | WP_010746839.1 |
| Enterococcus raffinosus | WP_218256688.1 |
| Enterococcus devriesei | WP_071861505.1 |
| Enterococcus hulanensis | WP_206919212.1 |
| Enterococcus hulanensis | WP_207114528.1 |
| Enterococcus hulanensis | WP_137665739.1 |
| Enterococcus gilvus | WP_221676391.1 |
| Lactococcus lactis subsp. lactis | KST89195.1 |
| Lactococcus garvieae | WP_074751524.1 |
| Enterococcus viikkiensis | WP_137614205.1 |
| Enterococcus malodoratus | WP_010743124.1 |
| Enterococcus sp. | HCM84609.1 |
| Enterococcus raffinosus | WP_216408036.1 |
| Enterococcus | WP_090403742.1 |
| Lactococcus sp. DD01 | WP_061414724.1 |
| Lactococcus garvieae | WP_165705621.1 |
| Lactococcus garvieae | WP_019299630.1 |
| Lactococcus garvieae IPLA 31405 | EIT65846.1 |
| Lactococcus sp. LG592 | WP_206887855.1 |
| Lactococcus petauri | WP_165717197.1 |
| Lactococcus | WP_017370980.1 |
| Lactococcus garvieae | WP_019335802.1 |
| Lactococcus petauri | WP_165719915.1 |
| Lactococcus petauri | WP_165707062.1 |
| Lactococcus petauri | WP_200553948.1 |
| Enterococcus sp. MJM 16 | WP_207109874.1 |
| Lactococcus garvieae | WP_040087086.1 |
| Lactococcus garvieae | WP_213439418.1 |
| Lactococcus | WP_117517726.1 |
| Lactococcus sp. LG1267 | WP_206921817.1 |
| Lactococcus petauri | WP_173817259.1 |
| Enterococcus sp. 669A | WP_207672175.1 |
| Enterococcus | WP_002287851.1 |
| Enterococcus faecium | NTK03502.1 |
| Enterococcus faecium | WP_077149154.1 |
| Enterococcus | WP_070828483.1 |
| Enterococcus faecium | HBM6588866.1 |
| Lactococcus petauri | WP_213482149.1 |
| Enterococcus faecium | WP_049141904.1 |
| Enterococcus faecium | WP_072538828.1 |
| Enterococcus faecium | EGP4780386.1 |
| Enterococcus faecium | WP_002323137.1 |
| Enterococcus faecium | WP_071242958.1 |
| Enterococcus | WP_002309701.1 |
| Enterococcus | WP_016853129.1 |
| Enterococcus faecium | WP_223617373.1 |
| Lactococcus cremoris | KZK07847.1 |
| Enterococcus faecium | WP_152133493.1 |
| Enterococcus | WP_002338601.1 |
| Enterococcus faecium | WP_107601719.1 |
| Enterococcus pallens | WP_010755318.1 |
| Enterococcus faecium | WP_107532024.1 |
| Enterococcus faecium | WP_158182420.1 |
| Enterococcus faecium | WP_002328692.1 |
| Lactococcus sp. | HAP15015.1 |
| Enterococcus hirae | WP_128479507.1 |
| Bacteria | WP_070544312.1 |
| Enterococcus faecium | WP_024635956.1 |
| Enterococcus faecium | WP_200781749.1 |
| Enterococcus faecium | EGP4854449.1 |
| Enterococcus mundtii | WP_104776415.1 |
| Enterococcus faecium | NTQ68095.1 |
| Enterococcus faecium | WP_107572576.1 |
| Lactococcus garvieae | WP_176490340.1 |
| Lactococcus garvieae | WP_202230365.1 |
| Enterococcus faecium | HAP8143250.1 |
| Enterococcus sp. 665A | WP_207702080.1 |
| Enterococcus faecium | WP_061344081.1 |
| Enterococcus faecium | WP_159037555.1 |
| Enterococcus faecium | WP_156249593.1 |
| Enterococcus gallinarum | WP_228160064.1 |
| Enterococcus | WP_005472579.1 |
| Enterococcus faecium | WP_002312492.1 |
| Enterococcus gallinarum | WP_219645506.1 |
| Enterococcus faecium | NTR43260.1 |
| Enterococcus faecium | MBK4849383.1 |
| Enterococcus gallinarum | MBS5961617.1 |
| Enterococcus gallinarum | WP_195493436.1 |
| Enterococcus gallinarum | WP_155852787.1 |
| Enterococcus gallinarum | WP_003127279.1 |
| Enterococcus faecium | MBK4788989.1 |
| Enterococcus faecium | HAQ5649929.1 |
| Enterococcus faecium | HAP8910317.1 |
| Enterococcus gallinarum | WP_103300086.1 |
| Enterococcus gallinarum | WP_208775262.1 |
| Enterococcus gallinarum | HJE79226.1 |
| Enterococcus faecium | HAP8972929.1 |
| Enterococcus faecium | HBM6451841.1 |
| Enterococcus faecium | HAZ5368934.1 |
| Enterococcus faecium | HAP8483481.1 |
| Enterococcus faecium | WP_104771920.1 |
| Enterococcus faecium | HAP8530966.1 |
| Enterococcus faecium | HAP8051565.1 |
| Enterococcus faecium | HAP7802562.1 |
| Enterococcus faecium | WP_200770494.1 |
| Enterococcus faecium | HBM6594778.1 |
| Enterococcus faecium | WP_196848605.1 |
| Enterococcus faecium | MBK4854836.1 |
| Enterococcus faecium | WP_002294522.1 |
| Enterococcus faecium | MBK4759248.1 |
| Enterococcus faecium | HAQ4565693.1 |
| Enterococcus faecium | HAP9519878.1 |
| Enterococcus faecium | HAR1789897.1 |
| Lactococcus lactis subsp. cremoris GE214 | KEY62214.1 |
| Lactococcus lactis subsp. cremoris TIFN5 | EQC55186.1 |
| Enterococcus sp. 3H8_DIV0648 | OTO21912.1 |
| Enterococcus asini | WP_118341330.1 |
| Lactococcus cremoris | BBC76182.1 |
| Lactococcus cremoris | WP_061778065.1 |
| Enterococcus faecium | HBA0799696.1 |
| Enterococcus faecium | HAP8944680.1 |

In embodiments, a GadC glutamate/GABA antiporter expressed in a genetically modified probiotic bacterium of the disclosure (e.g., L. lactis) includes an L. lactis GadC having an amino acid sequence as set forth in SEQ ID NO: 2.

In embodiments, a GadC glutamate/GABA antiporter expressed in a genetically modified probiotic bacterium of the disclosure (e.g., L. lactis) has a sequence having at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or at least 99.9% sequence identity to an L. lactis GadC amino acid sequence as set forth in SEQ ID NO: 2, provided the glutamate/GABA antiporter retains functional activity. In embodiments, a glutamate/GABA antiporter having functional activity can import extracellular glutamate into a cell and/or export GABA from inside a cell to the extracellular space.

In embodiments, a GadC glutamate/GABA antiporter expressed in a genetically modified probiotic bacterium of the disclosure (e.g., L. lactis) is encoded by an L. lactis gadC gene having a nucleic acid sequence as set forth in SEQ ID NO: 4.

In embodiments, a gadC gene expressed in a genetically modified probiotic bacterium of the disclosure (e.g., L. lactis) has a sequence having at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or at least 99.9% sequence identity to a nucleic acid sequence encoding an L. lactis GadC glutamate/GABA antiporter as set forth in SEQ ID NO: 2, provided the gadC gene encodes a glutamate/GABA antiporter having functional activity. In embodiments, a glutamate/GABA antiporter having functional activity can import extracellular glutamate into a cell and/or export GABA from inside a cell to the extracellular space.

In embodiments, a gadC gene expressed in a genetically modified probiotic bacterium of the disclosure (e.g., L. lactis) has a sequence having at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or at least 99.9% sequence identity to a nucleic acid sequence as set forth in SEQ ID NO: 4, provided the gadC gene encodes a glutamate/GABA antiporter having functional activity. In embodiments, a glutamate/GABA antiporter having functional activity can import extracellular glutamate into a cell and/or export GABA from inside a cell to the extracellular space.

In embodiments, an L. lactis GadC glutamate/GABA antiporter having at least 80% sequence identity to an L. lactis GadC amino acid sequence as set forth in SEQ ID NO: 2 encodes a functional GadC glutamate/GABA antiporter. In embodiments, an L. lactis gadC nucleic acid sequence having at least 80% sequence identity to a nucleic acid encoding a sequence as set forth in SEQ ID NO: 2 encodes a functional GadC glutamate/GABA antiporter. In embodiments, an L. lactis gadC nucleic acid sequence having at least 80% sequence identity to a nucleic acid sequence as set forth in SEQ ID NO: 4 encodes a functional GadC glutamate/GABA antiporter. A functional GadC glutamate/GABA antiporter can import glutamate from outside of a cell into the cell and/or export GABA from inside a cell to outside the cell.

In embodiments, a genetically modified probiotic bacterium of the disclosure (e.g., L. lactis) includes a genetic construct including an expression cassette having a gadB gene and a gadC gene that allows expression of these genes to enhance GABA production. In embodiments, the gadB and gadC genes in the expression cassette are operably linked to a heterologous promoter. In embodiments, the heterologous promoter includes a constitutive promoter. In embodiments, the constitutive promoter includes a P8 short promoter from L. lactis (Zhu et al. FEMS Microbiol Lett 2015, 362) with a sequence as set forth in SEQ ID NO: 5.

In embodiments, the constitutive promoter includes a P8 promoter from L. lactis (Zhu et al. FEMS Microbiol Lett 2015, 362) with a sequence as set forth in SEQ ID NO: 6.

In embodiments, the heterologous promoter includes an inducible promoter.

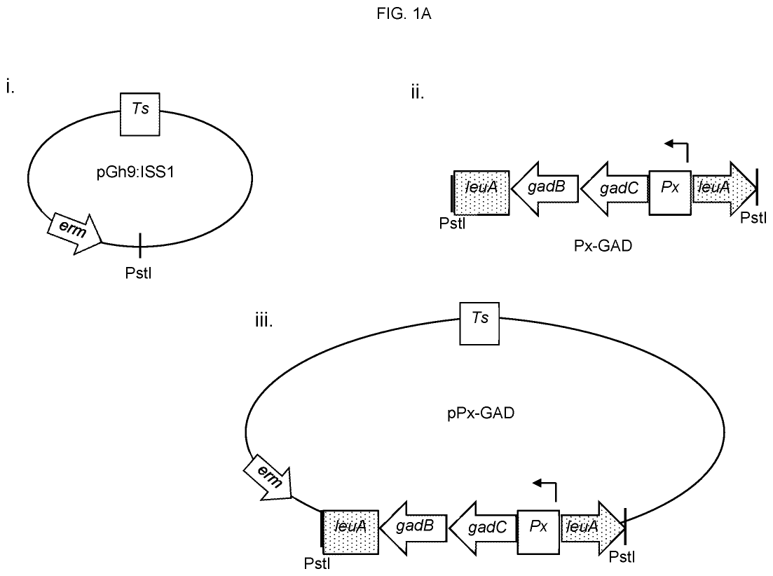

In embodiments, a genetic construct including an expression cassette having heterologous gadB and gadC genes includes homology arms 5′ and 3′ of the expression cassette for integration into the genome of a bacterium. In embodiments, the genetic construct is integrated at any location in the genome that does not affect viability of the bacterium. For example, a genetically modified probiotic bacterium (e.g., L. lactis) of the present disclosure can have its endogenous leuA gene, encoding 2-isopropylmalate synthase, disrupted and replaced with a genetic construct including a heterologous gadB gene and a heterologous gadC gene. See FIGS. 1A, 1B.

The leuA gene encodes an enzyme that catalyzes the condensation of the acetyl group of acetyl-CoA with 3-methyl-2-oxobutanoate (2-oxoisovalerate) to form 3-carboxy-3-hydroxy-4-methylpentanoate (2-isopropylmalate) as a first step to synthesizing leucine. In embodiments, the LeuA protein includes an L. lactis LeuA protein having an amino acid sequence as set forth in SEQ ID NO: 7 (UniProt ID Q02141).

In embodiments, the leuA gene includes an L. lactis leuA gene having a nucleic acid sequence as set forth in SEQ ID NO: 8.

In embodiments, the homology arm 5′ to the expression cassette having heterologous gadB and gadC genes includes a sequence as set forth in SEQ ID NO: 9, which is homologous to coding sequence of the endogenous L. lactis leuA gene.

In embodiments, the homology arm 5′ to the expression cassette having heterologous gadB and gadC genes includes a sequence having at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, or at least 99.9% sequence identity to a sequence as set forth in SEQ ID NO: 9, provided the homology arm retains its function. In embodiments, a homology arm 5′ to the expression cassette having heterologous gadB and gadC genes is functional when it, along with a functional 3′ homology arm, allows integration of the expression cassette into a target genome. In embodiments, the homology arm 5′ to the expression cassette having heterologous gadB and gadC genes includes a sequence as set forth in SEQ ID NO: 9.

In embodiments, the homology arm 3′ to the expression cassette having heterologous gadB and gadC genes includes a sequence as set forth in SEQ ID NO: 10, which is homologous to coding sequence of the endogenous L. lactis leuA gene.

In embodiments, the homology arm 3′ to the expression cassette having heterologous gadB and gadC genes includes a sequence having at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, or at least 99.9% sequence identity to a sequence as set forth in SEQ ID NO: 10, provided the homology arm retains its function. In embodiments, a homology arm 3′ to the expression cassette having heterologous gadB and gadC genes is functional when it, along with a functional 5′ homology arm, allows integration of the expression cassette into a target genome. In embodiments, the homology arm 3′ to the expression cassette having heterologous gadB and gadC genes includes a sequence as set forth in SEQ ID NO: 10.

In embodiments, the heterologous gadB and gadC genes of the genetic construct are oriented such that they are transcribed in the opposite direction from (anti-parallel to) transcription of the endogenous gene that is disrupted (see FIGS. 1A, 1B). In embodiments, the endogenous gene is leuA.

In embodiments, a genetic construct of the present disclosure includes an expression cassette, wherein the expression cassette includes a gene encoding a GAD enzyme and a gene encoding a glutamate/GABA antiporter operably linked to a promoter. In embodiments, a genetic construct of the present disclosure includes from 5′ to 3′: (a) a promoter; (b) a gadC gene encoding a glutamate/GABA antiporter; and (c) a gadB gene encoding a GAD enzyme. In embodiments, the promoter is a constitutive promoter. In embodiments, the promoter is an inducible promoter. In embodiments, the genetic construct further includes a selectable marker. In embodiments, the selectable marker encodes an erythromycin resistance gene. In embodiments, the genetic construct further includes a homology arm upstream (5′) of the expression cassette and a homology arm downstream (3′) of the expression cassette to enable integration into the genome of a probiotic bacterium at target sequences homologous to the homology arms.

In embodiments, a genetic construct of the present disclosure includes a sequence as set forth in SEQ ID NO: 11.

In embodiments, the genetically modified probiotic bacteria include endogenous glutamic acid decarboxylase and glutamate/GABA antiporter genes. Therefore, the heterologous gadB and gadC genes of the genetic construct add an extra copy of each gene in each genetically modified probiotic bacterium and confers enhanced GABA production as compared to probiotic bacteria of the same species that have not been genetically modified or as compared to probiotic bacteria of the same species that have been genetically modified to include an empty plasmid (i.e. the starting plasmid that is used to construct the genetic construct to express gadB and gadC but without the heterologous gadB and gadC genes).

The genetically modified probiotic bacteria have enhanced production of GABA as compared to a control. In embodiments, the genetically modified probiotic bacteria produce an amount of GABA that includes 2-fold to 200-fold greater, or 2-fold to 150-fold greater, or 2-fold to 120-fold greater, or 2-fold to 20-fold greater, or 5-fold to 20-fold greater, as compared to an amount of GABA produced by a control. In embodiments, the genetically modified probiotic bacteria produce an amount of GABA that includes 2-fold, 3-fold, 4-fold, 5-fold, 6-fold, 7-fold, 8-fold, 9-fold, 10-fold, 11-fold, 12-fold, 13-fold, 14-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 105-fold, 110-fold, 120-fold, 130-fold, 140-fold, 150-fold, 160-fold, 170-fold, 180-fold, 190-fold, 200-fold, or more as compared to an amount of GABA produced by a control. In embodiments, the genetically modified probiotic bacteria produce an amount of GABA that includes 500 ng/ml GABA to 60,000 ng/mL GABA, or 500 ng/ml GABA to 50,000 ng/ml GABA, or 500 ng/ml GABA to 20,000 ng/mL GABA. In embodiments, the genetically modified probiotic bacteria produce an amount of GABA that includes 500 ng/ml GABA, 550 ng/ml GABA, 600 ng/ml GABA, 650 ng/ml GABA, 700 ng/ml GABA, 750 ng/ml GABA, 800 ng/ml GABA, 850 ng/ml GABA, 900 ng/ml GABA, 950 ng/ml GABA, 1000 ng/mL GABA, 1100 ng/mL GABA, 1200 ng/ml GABA, 1300 ng/ml GABA, 1400 ng/ml GABA, 1500 ng/ml GABA, 1600 ng/mL GABA, 1700 ng/ml GABA, 1800 ng/ml GABA, 1900 ng/mL GABA, 2000 ng/ml GABA, 3000 ng/ml GABA, 4000 ng/ml GABA, 5000 ng/mL GABA, 6000 ng/ml GABA, 7000 ng/ml GABA, 8000 ng/ml GABA, 9000 ng/ml GABA, 10,000 ng/mL GABA, 11,000 ng/mL GABA, 12,000 ng/ml GABA, 13,000 ng/mL GABA, 14,000 ng/ml GABA, 12,000 ng/ml GABA, 13,000 ng/mL GABA, 14,000 ng/ml GABA, 15,000 ng/ml GABA, 16,000 ng/ml GABA, 17,000 ng/ml GABA, 18,000 ng/ml GABA, 19,000 ng/mL GABA, 20,000 ng/ml GABA, 30,000 ng/ml GABA, 40,000 ng/ml GABA, 50,000 ng/ml GABA, 60,000 ng/mL GABA, or more. In embodiments, a control includes probiotic bacteria of the same genus or species that have not been genetically modified. In embodiments, a control includes probiotic bacteria of the same genus or species that have been genetically modified to include a control plasmid (i.e. the same backbone plasmid used in the genetically modified probiotic bacteria producing GABA but not including the expression cassette having a gene encoding a GAD enzyme and a gene encoding a glutamate/GABA antiporter). In embodiments, a control includes a probiotic bacteria of the same genus or species that have been genetically modified to include an unrelated control genetic construct (i.e. a genetic construct that includes an expression cassette unrelated to GABA production). In embodiments, a genetically modified probiotic bacteria producing GABA can produce 2-fold to 30-fold more, or 10-fold to 24-fold more, or 2-fold to 20-fold more, or 2-fold to 10-fold more, or 2-fold to 8-fold more, or 2-fold to 4-fold more GABA when glutamic acid/glutamate is provided in culture as compared to genetically modified probiotic bacteria producing GABA cultured in media not supplemented with glutamic acid/glutamate. In embodiments, the glutamic acid/glutamate is provided in culture as glutamic acid HCl. In embodiments, the glutamic acid/glutamate (e.g., glutamic acid HCl) is provided in culture at a concentration of 50 mM, 100 mM, 150 mM, or 200 mM.

Genetic modification of bacteria. As used herein, the term “genetically modified” or “genetically engineered” refers to the addition of extra genetic material in the form of DNA or RNA into the total genetic material in a cell or modification of the genome of a cell. In embodiments, the extra genetic material remains separate from the genome of the cell (e.g., the extra genetic material resides on a plasmid or vector that exists in the cell as an entity separate from the cell's genome). In embodiments, the genetic modification results in the genome containing insertions, deletions, mutations, and/or rearrangements of the genomic DNA after introduction of extra genetic material as compared to a cell that is not genetically modified. For clarity the term “genetically modified” or “genetically engineered” also includes the removal of DNA from a genome without the insertion of extra genetic material. The term “genetically modified” or “genetically engineered” includes artificial manipulation of a cell to alter the genotype of that cell to modulate physiology or function of that cell, such as expressing a heterologous gene product, deleting endogenous genes, and/or altering regulation or expression of endogenous genes. The extra genetic material can be derived from the same organism as the genome it is inserted into or it can be derived from a different genome or be synthetic. The terms “genetically modified bacteria”, “genetically engineered bacteria”, “modified bacteria”, and “engineered bacteria” are used interchangeably. The term “genetically modified” or “genetically engineered” also refers to multiple genetic modifications, e.g. 2, 3, 4, 5, 6, 7, 8, 9, 10 or more genetic modifications, for example, a bacterium which has a heterologous gene introduced for expression of a glutamic acid decarboxylase (gadB), and another heterologous gene introduced for expression of a glutamate/GABA antiporter (gadC).

The term “bacterium” or “bacteria” refers to a single-cell microorganism or a class of single-cell microorganisms found almost everywhere on Earth and inside and outside of a body. In embodiments, bacteria include four common forms: Coccus form, which are spherical bacteria (e.g., Streptococcus pneumoniae); Bacillus form, which are rod-shaped bacteria (e.g., Lactobacillus acidophilus); Spirilla form, which are spiral-shaped bacteria (e.g., Spirillum volutans); and Vibrio form, which are comma-shaped bacteria (e.g., Vibrio cholerae). In embodiments, a bacteria can be beneficial or pathogenic to a host organism that the bacteria colonize. In embodiments, genetically modified probiotic bacteria described herein are beneficial to a host organism. In embodiments, a bacteria or bacterium can refer to a single bacterial cell (e.g., a L. lactis bacteria includes a single L. lactis cell). In embodiments, bacteria can refer to a population of bacterial cells. In embodiments, bacteria can refer to bacteria of a taxa, a class, a genus, a species, etc. (e.g. a Lactococcus bacteria includes a microorganism belonging to the Lactococcus genus).

The term “heterologous” refers to a molecule (e.g., nucleic acid, gene, RNA, protein) that originates outside bacteria and is introduced into bacteria by genetic engineering. In embodiments, a heterologous molecule can include sequences that are not native to bacteria to which the heterologous molecule is introduced; the heterologous molecule is synthesized outside the bacteria and introduced into the bacteria. For example, genetically modified L. lactis bacteria of the disclosure can include extra copies of gadB and/or gadC genes that are from an organism other than L. lactis. In embodiments, a heterologous molecule can include sequences that are native to a bacterium to which the heterologous molecule is introduced, but the heterologous molecule is synthesized outside the bacterium and introduced into the bacterium. For example, the disclosure includes a genetically modified L. lactis including a heterologous gene encoding L. lactis gadB and a heterologous gene encoding L. lactis gadC. The introduced gadB and gadC genes are native to L. lactis but are synthesized or cloned and introduced into L. lactis as part of an expression cassette such that the expression of the introduced L. lactis gadB and L. lactis gadC is driven by a heterologous promoter.

The term “endogenous” refers to a molecule (e.g., nucleic acid, gene, RNA, protein) that is naturally occurring or naturally produced in a given bacterium. For example, genes or proteins found naturally in bacteria are genes or proteins that are endogenous to the bacteria. A genetically modified L. lactis bacterium of the present disclosure includes endogenous gadB and gadC genes in addition to having an extra copy of the L. lactis gadB gene and an extra copy of the L. lactis gadC gene introduced on a plasmid. The term “native” can be used interchangeably with “endogenous”. In embodiments, the term “endogenous” can refer to a wild-type version of a molecule in a given bacterium.

In embodiments, the term “gene” refers to a nucleic acid sequence (used interchangeably with polynucleotide or nucleotide sequence) that encodes, e.g., a protein associated with production of GABA as described herein. This definition includes various sequence polymorphisms, mutations, and/or sequence variants wherein such alterations do not substantially affect the function of the encoded protein. The nucleic acid sequences can include both the full-length nucleic acid sequences as well as non-full-length sequences derived from a full-length protein coding sequence. The sequences can also include degenerate codons of the native sequence or sequences that can be introduced to provide codon preference in specific bacteria. In embodiments, the term “gene” can include not only coding sequences but also regulatory regions such as promoters, enhancers, 5′ UTR, 3′UTR, termination regions, and non-coding regions. Gene sequences encoding a molecule can be DNA or RNA that directs the expression of the molecule. These nucleic acid sequences can be a DNA strand sequence that is transcribed into RNA or an RNA sequence that is translated into protein. An essential gene is an endogenous (e.g., endogenous to a bacterium) or heterologous gene (e.g., a selectable marker or gene of interest) that produces a polypeptide (e.g., an essential protein) that is necessary for the growth and/or viability of a bacterium.

“Encoding” refers to the property of specific sequences of nucleotides in a gene, such as a complementary DNA (cDNA), or a messenger RNA (mRNA), to serve as templates for synthesis of other macromolecules such as a defined sequence of amino acids or a functional polynucleotide (e.g., siRNA). In embodiments, a gene encodes or codes for a protein if the gene is transcribed into mRNA and translation of the mRNA produces the protein in a cell or other biological system. A “gene sequence encoding a protein” includes all nucleotide sequences that are degenerate versions of each other and that code for the same amino acid sequence or amino acid sequences of substantially similar form and function.

A “gene deletion”, “gene disruption”, or “gene knockout” refers to a combination of genetic techniques that can render a specific gene inoperable or inactive. In particular embodiments, a gene deletion reduces or eliminates expression of a polypeptide encoded by the gene. In particular embodiments, the expression of the gene is substantially reduced or eliminated. Substantially reduced means that the expression of a gene is reduced by at least 80%, at least 90%, at least 95%, or at least 98% when compared to an endogenous level of expression of the gene. Expression of a gene can be determined by a suitable technique (e.g., by measuring transcript or expressed protein levels). In embodiments, a gene can be deleted by disabling an endogenous promoter, operon or regulatory element that is essential for transcription or translation of the gene. In embodiments, a gene is deleted by introducing one or more mutations that disable the function of a protein encoded by the gene. In embodiments, a gene is disrupted or deleted when a portion of the gene or the complete gene is removed from the genome of a bacterium. In embodiments, an endogenous gene is deleted by replacing a part of the gene or the complete gene with a different gene, with a genetic construct, and/or with a selectable marker (e.g., antibiotic selectable marker, auxotrophic selectable marker). For example, a genetically modified probiotic bacterium (e.g., L. lactis) of the present disclosure can have its endogenous leuA gene disrupted and replaced with a genetic construct including a gadB gene and a gadC gene. In embodiments, portions of the coding sequence of the leuA gene remain in the genome, but the disruption results in no or substantially reduced expression of the leuA protein.

Any suitable technique can be used to generate a gene deletion in a bacterium. In embodiments, a gene deletion in a bacterium can be mediated by a gene editing system including Clustered Regularly Interspaced Short Palindromic Repeats/CRISPR-associated (CRISPR/Cas), transcription activator-like effector nucleases (TALENs), zinc-finger nucleases (ZFNs), and meganucleases.

In embodiments, deleting an endogenous gene and replacing it with another gene or a genetic construct in a bacterium can occur by homologous recombination. Homologous recombination includes introducing a genetic construct into a bacterium, where the genetic construct includes homology arms having homology to target sequences of the endogenous gene to be deleted. In embodiments, the genetic construct includes a non-homologous polynucleotide flanked by two polynucleotide regions of homology (i.e., the upstream (5′) and downstream (3′) homology arms), such that homologous recombination between target sequences of the endogenous gene to be deleted and the two flanking homology arms results in insertion of the non-homologous polynucleotide at the target region (see FIGS. 1A, 1B). In embodiments, the target sequence homologous to the upstream homology arm includes sequence 5′ of the coding sequence and/or coding sequence of the endogenous gene to be deleted. In embodiments, the target sequence homologous to the downstream homology arm includes sequence 3′ of the coding sequence and/or coding sequence of the endogenous gene to be deleted. One of skill in the art will recognize that the upstream and downstream homology arms can have homology to target sequences such that less than the full-length coding sequence of a gene is deleted, a combination of a portion of the full-length coding sequence and sequences upstream (5′) and/or downstream (3′) of the coding sequence is deleted, a combination of the full-length coding sequence and sequences upstream (5′) and/or downstream (3′) of the coding sequence is deleted, or any other variation on deletion of a gene, as long as expression of the gene is reduced or eliminated. In embodiments, a homology arm includes sequence having at least 50% sequence identity to a target sequence with which homologous recombination is desired. In embodiments, a homology arm includes sequence having at least 60%, at least 70%, at least 80%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to a target sequence. In embodiments, each homology arm can include 100 to 1000 nucleotides (nt), 200 to 800 nt, or 200 to 500 nt. In embodiments, each homology arm can include 100 nt, 150 nt, 200 nt, 250 nt, 300 nt, 350 nt, 400 nt, 450 nt, 500 nt, 750 nt, 1000 nt, 1250 nt, 1500 nt, 2000 nt, 2500 nt, 3000 nt, or more. In embodiments, the non-homologous polynucleotide flanked by the upstream and downstream homology arms includes a promoter, a gene, a terminator, a selectable marker, a counter-selectable marker, or a combination thereof. In embodiments, disruption of an endogenous gene in a bacterium by homologous recombination includes deletion of the endogenous gene without any heterologous sequences inserted at the target sequences. In embodiments, disruption of an endogenous gene in a bacterium by homologous recombination includes deletion of the endogenous gene and concurrent insertion of heterologous sequences, such as heterologous expression cassettes including selectable or counter-selectable markers, at the target sequences. In embodiments, disruption of an endogenous gene in a bacterium by homologous recombination reduces or eliminates expression of the endogenous gene but portions of the endogenous gene remain in the genome while other portions of the endogenous gene are deleted.

The terms “peptide,” “oligopeptide,” “polypeptide,” “polyprotein,” and “protein” are used interchangeably herein and refer to a polymeric form of amino acids of any length, which can include coded and non-coded amino acids, chemically or biochemically modified or derivatized amino acids, and polypeptides having modified peptide backbones.

The term “recombinant” refers to a particular DNA or RNA sequence that is the product of various combinations of cloning, restriction, and/or ligation steps resulting in a construct having a structural coding or non-coding sequence distinguishable from homologous sequences found in natural systems. Generally, DNA sequences encoding the structural coding sequence can be assembled from cDNA fragments and short oligonucleotide linkers, or from a series of oligonucleotides, to provide a synthetic gene which is capable of being expressed in a recombinant transcriptional unit. Such sequences can be provided in the form of an open reading frame uninterrupted by internal non-translated sequences, or introns. Genomic DNA including the relevant sequences could also be used. Sequences of non-translated DNA can be present 5′ or 3′ from the open reading frame, where such sequences do not interfere with manipulation or expression of the coding regions. In embodiments, the term “recombinant” polynucleotide or nucleic acid refers to one which is not naturally occurring or is made by the artificial combination of two otherwise separated segments of sequence. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions.

Similarly, a “recombinant polypeptide” refers to a polypeptide or polyprotein which is not naturally occurring or is made by the artificial combination of two otherwise separated segments of amino acid sequences. This artificial combination can be accomplished by standard techniques of recombinant DNA technology, i.e., a recombinant polypeptide can be encoded by a recombinant polynucleotide. Thus, a recombinant polypeptide is an amino acid sequence encoded by all or a portion of a recombinant polynucleotide.

A “genetic construct” includes a recombinant nucleic acid, generally recombinant DNA, which has been generated for the purpose of the expression of a specific nucleotide sequence(s) or is to be used in the construction of other recombinant nucleotide sequences. In embodiments, the term genetic construct includes plasmids and vectors. In embodiments, a genetic construct can be circular or linear. Genetic constructs can include, for example, an origin of replication, a multicloning site, and/or a selectable marker. In embodiments, a genetic construct includes nucleic acid (e.g., homology arms) to enable deletion of an endogenous gene in a bacterium. In embodiments, a genetic construct includes an expression cassette. In embodiments, an expression cassette of the disclosure includes: (a) a heterologous promoter; and (b) a heterologous gene encoding a GAD enzyme and a heterologous gene encoding a glutamate/GABA antiporter. In embodiments, the genes in an expression cassette are in an operon. An operon refers to a functioning unit of nucleic acid including a cluster of genes that are operably linked to a single promoter and thus are transcribed together. In embodiments, a genetic construct of the disclosure can further include a selectable marker.

A genetic construct of the disclosure can include a gene encoding a selectable marker and/or counter-selectable marker. In embodiments, cells expressing a selectable marker can grow in the presence of a selective agent or under a selective growth condition. Examples of selectable markers include antibiotic resistance markers (e.g., erythromycin resistance, chloramphenicol resistance, ampicillin resistance, carbenicillin resistance, kanamycin resistance, spectinomycin resistance, streptomycin resistance, tetracycline resistance, bleomycin resistance, and polymyxin B resistance), markers that complement an essential gene (e.g., alanine auxotrophy (alr), diaminopimelic acid auxotrophy (dapD), thymidine auxotrophy (thyA), proline auxotrophy (proBA), glycine auxotrophy (glyA), carbon source auxotrophy (TpiA)), chemical resistance (e.g., tellurite resistance, Fabl for triclosan resistance, bialaphos herbicide resistance, mercury resistance, arsenic resistance), and visual markers (e.g., green fluorescent protein (GFP), luciferase, β-galactosidase (lacZ)). In embodiments, a genetic construct of the disclosure includes an erythromycin resistance erm gene encoding a methylase. In embodiments, cells can be positively selected that have lost expression of a counter-selectable marker (i.e. cells expressing a counter-selectable marker are selected against). Examples of genes encoding counter-selectable markers include: sacB (gene encoding levansucrase that converts sucrose to levans, which is harmful to bacteria); rpsL (strA) (encodes the ribosomal subunit protein (S12) target of streptomycin); tetAR (confers sensitivity to lipophilic compounds such as fusaric and quinalic acids); pheS (encodes the a subunits of Phe-tRNA synthetase, which renders bacteria sensitive to p-chlorophenylalanine, a phenylalanine analog); thyA (encodes thymidilate synthetase, which confers sensitivity to trimethoprim and related compounds); lacY (encodes lactose permease, which renders bacteria sensitive to t-o-nitrophenyl-β-D-galactopyranoside); gata-1 (encodes a zinc finger DNA-binding protein which inhibits the initiation of bacterial replication); and ccdB (encodes a cell-killing protein which is a potent poison of bacterial gyrase).

The term “expression cassette” includes a polynucleotide construct that is generated recombinantly or synthetically and includes regulatory sequences operably linked to a selected polynucleotide to facilitate expression of the selected polynucleotide in bacteria. For example, the regulatory sequences can facilitate transcription of the selected polynucleotide in bacteria, or transcription and translation of the selected polynucleotide in bacteria. In embodiments, the expression cassette includes an operon, a cluster of genes under the control of a common promoter. Therefore, genes within an operon are expressed together. In embodiments, the expression cassette is introduced as part of a genetic construct into probiotic bacteria. In embodiments, the expression cassette is subsequently integrated into the genome of a probiotic bacterium. A heterologous expression cassette can be integrated into the genome of the probiotic bacterium by any method known to one of skill in the art, including by homologous recombination.

The term “overexpression” refers to a greater expression level of a gene encoding a given polypeptide in genetically modified probiotic bacteria as compared to expression in wild type probiotic bacteria at any developmental or temporal stage for the gene. In embodiments, overexpression can occur when the gene is under the control of a strong promoter (e.g., the P8 promoter). Overexpression can also occur under the control of an inducible promoter. In particular embodiments, overexpression can occur in genetically modified probiotic bacteria where endogenous expression of a given polypeptide normally occurs, but such normal expression is at a lower level. In embodiments, overexpression can also occur in genetically modified probiotic bacteria lacking endogenous expression of a given polypeptide. Overexpression thus results in a greater than normal production or “overproduction” of a given polypeptide in genetically modified probiotic bacteria. Increased activity of a protein can result from overexpression or the modification of a peptide or a polypeptide such that it causes the peptide or polypeptide to have a higher activity. For example, in the case where a polypeptide is an enzyme, the enzyme can have an increased catalytic turnover rate.

In embodiments, a genetically modified probiotic bacterium includes a gene where expression of the gene is regulated by a promoter and/or regulatory elements. A promoter and/or regulatory elements are often introduced at a suitable location relative to a gene of interest. For example, a promoter (e.g., an inducible promoter) is often placed 5′ of a transcription start site of a gene of interest. In embodiments, a nucleic acid includes a promoter and/or regulatory elements necessary to drive the expression of a gene (e.g., a heterologous gene or an endogenous gene). A promoter can be an endogenous promoter, a heterologous promoter, or a combination thereof. In embodiments, a promoter includes a constitutive promoter.

In embodiments, a constitutive promoter includes L. lactis P8 promoter. In embodiments, a constitutive promoter includes L. lactis P8 promoter as set forth in SEQ ID NO: 5 or SEQ ID NO: 6. In embodiments, the P8 promoter includes the P8 short promoter as set forth in SEQ ID NO: 5. In embodiments, the P8 promoter includes the P8 promoter as set forth in SEQ ID NO: 6. In embodiments, a constitutive promoter includes L. lactis P2 promoter. In embodiments, a constitutive promoter includes L. lactis P2 promoter as set forth in SEQ ID NO: 12. In embodiments, a constitutive promoter includes L. lactis P5 promoter. In embodiments, a constitutive promoter includes L. lactis P5 promoter as set forth in SEQ ID NO: 13. In embodiments, a constitutive promoter includes L. lactis P1 promoter. In embodiments, a constitutive promoter includes L. lactis P1 promoter as set forth in SEQ ID NO: 23. In embodiments, a constitutive promoter includes L. lactis P3 promoter. In embodiments, a constitutive promoter includes L. lactis P3 promoter as set forth in SEQ ID NO: 24. In embodiments, a constitutive promoter includes L. lactis P4 promoter. In embodiments, a constitutive promoter includes L. lactis P4 promoter as set forth in SEQ ID NO: 25. In embodiments, a constitutive promoter includes L. lactis P6 promoter. In embodiments, a constitutive promoter includes L. lactis P6 promoter as set forth in SEQ ID NO: 26. In embodiments, a constitutive promoter includes L. lactis P7 promoter. In embodiments, a constitutive promoter includes L. lactis P7 promoter as set forth in SEQ ID NO: 27. In embodiments, a constitutive promoter includes L. lactis P32 and L. lactis P45 promoters (Vossen et al. Appl. Environ. Microbiol. 1987, 53, 2452-2457; MacCormick et al. FEMS Microbiol Lett 1995, 127, 105-109; Takala et al. Appl Microbiol Biotechnol 2002, 59, 467-471; Zhu et al. FEMS Microbiol Lett 2015, 362). In embodiments, a constitutive promoter includes LacA promoter and PPepN promoter (Wegkamp et al. Applied and Environmental Microbiology, 2007, 73, 2673-2681; Constitutive Gene Expression System for Lactococcus lactis and Other Lactic Acid Bacteria. Handbook October 2016. MoBiTec GmBH, Goettingen, Germany). In embodiments, a constitutive promoter includes P6C and P13C promoters (Jeong et al. Food Microbiology, 2006, 23 (1), 82-89). In embodiments, a constitutive promoter includes a PTS-IIC promoter (Ogaugwu et al. Biotechnology Reports, 2018, 17, 86-92). In embodiments, a constitutive promoter includes variants of an L. lactis noxE promoter (Guo et al. PLOS ONE, 2012 7, e36296). In embodiments, a constitutive promoter includes constitutive synthetic promoters (Jensen and Hammer. Biotechnol. Bioeng. 1998, 58, 191-195; Jensen and Hammer. Appl. Environ. Microbiol. 199864, 82-87; Lindholm and Palva. Biotechnol Lett 2009, 32, 131). In embodiments, the heterologous constitutive promoter is derived from Lactococcus genus. In embodiments, the heterologous constitutive promoter is derived from a lactic acid bacteria.

In embodiments, promoters described in U.S. Pat. No. 5,529,908A and Guo et al. PLOS ONE, 2012, 7 (4), e36296 can be used.

In embodiments, probiotic bacteria are genetically engineered to include a gene under the control of an inducible promoter. An inducible promoter is often a nucleic acid sequence that directs the conditional expression of a gene. An inducible promoter can be an endogenous promoter, a heterologous promoter, or a combination thereof. An inducible promoter can include an operon system. In embodiments, an inducible promoter requires the presence of a certain compound, nutrient, amino acid, sugar, peptide, protein or condition (e.g., light, oxygen, heat, cold) to induce gene activity (e.g., transcription). In embodiments, an inducible promoter includes one or more repressor elements. In embodiments, an inducible promoter including a repressor element requires the absence of a certain compound, nutrient, amino acid, sugar, peptide, protein or condition to induce gene activity (e.g., transcription). Any suitable inducible promoter, system, or operon can be used to regulate the expression of a gene. Non-limiting examples of inducible promoters include temperature inducible promoters (e.g., heat inducible PgroES promoter, heat inducible phage lambda pL promoter, heat inducible phage lambda pR promoter, cold inducible cspA promoter), lactose regulated systems (e.g., lactose operon systems), sugar regulated systems, metal regulated systems, steroid regulated systems, alcohol regulated systems, IPTG inducible systems (e.g., pLac promoter), arabinose regulated systems (e.g., arabinose operon systems, pBad promoter), synthetic amino acid regulated systems (e.g., see Rovner et al. Nature, 2015, 518 (7537), 89-93), fructose repressors, a tac promoter/operator (pTac), tryptophan promoters (e.g., Ptrp, induced by tryptophan depletion or by addition of β-indoleacrylic acid), alkaline phosphatase promoters (e.g., PhoA promoter induced by phosphate limitation), recA promoters (e.g., recA promoter induced by UV light), proU promoters (e.g., osmotically inducible proU promoter), cst promoters (e.g., cst promoter inducible by carbon starvation), tetA promoters (e.g., tetracycline inducible tetA promoter), cadA and cadR promoters (e.g., PcadA and PcadR induced by cadmium), nar promoters (e.g., nar promoter induced by oxygen), or combinations thereof.

In embodiments, a lactate-inducible p170 promoter can be used (Jorgensen et al. FEMS Microbiol Lett. 2014, 351 (2), 170-178). In embodiments, a nisin-inducible promoter can be used (Mierau and Kleerebezem. Applied Microbiology and Biotechnology, 2005, 9, 1-13; NICE® Expression System for Lactococcus lactis. The effective & easy-to-operate Nisin Controlled gene Expression system. Handbook November 2015. MoBiTec GmBH, Goettingen, Germany). In embodiments, an agmatine-controlled expression (ACE) system including a lactococcal agmatine-sensor/transcriptional activator AguR and its target promoter P_{aguB} can be used (Linares et al. Microb Cell Fact, 2015, 14, 208).

In embodiments, expression of a gene can be controlled in additional ways known to one of skill in the art including modifying: gene copy number, number of copies of transcription factors binding the promoter operably linked to the gene; transcription factor binding to the gene promoter; RNA polymerase binding affinity for the gene promoter; ribosome binding affinity for the RBS; mRNA decay rate; and protein decay rate (Brewster et al. (2012) PLOS Comput Biol 8 (12): e1002811). In embodiments, a promoter such as T7 can be regulated using a system with a temperature sensitive intein inserted in the protein sequence of T7 RNA polymerase (Korvin and Yadav (2018) Molecular Systems Design & Engineering 3 (3): 550-559). The polymerase is only active and able to drive gene expression when the intein is spliced out at the appropriate temperature.

The term “operably linked” refers to polynucleotide sequences or amino acid sequences placed into a functional relationship with one another. For instance, a promoter or enhancer is operably linked to a coding sequence if it regulates, or contributes to the modulation of, the transcription of the coding or non-coding sequence. In embodiments, regulatory sequences operably linked to a coding sequence are typically contiguous to the coding sequence. However, enhancers can function when separated from a promoter by up to several kilobases or more. Accordingly, some polynucleotide elements can be operably linked but not contiguous. In embodiments, a heterologous promoter or heterologous regulatory elements include promoters and regulatory elements that are not normally associated with a particular nucleic acid in nature.

A termination region can be provided by the naturally occurring or endogenous transcriptional termination region of the polynucleotide sequence encoding a protein of the disclosure. In embodiments, a genetic construct of the present disclosure includes, as a termination region, sequences downstream of the stop codon of the gadB gene. In embodiments, a genetic construct of the present disclosure includes, as a termination region, 177 base pairs downstream of the stop codon of the gadB gene. Alternatively, the termination region can be derived from a different source. For the most part, the source of the termination region is generally not considered to be critical to the expression of a recombinant protein and a wide variety of termination regions can be employed without adversely affecting expression.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, a process sometimes called “codon optimization” or “controlling for species codon bias.”

Optimized coding sequences containing codons preferred by a particular prokaryotic or eukaryotic host (see also, Murray et al. (1989) Nucl. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al. (1996) Nucl. Acids Res. 24:216-218).

In embodiments, a genetic construct of the disclosure can be propagated in vitro in a host cell suitable for replication of the genetic construct. Host cells can include bacterial cells, mammalian cells, yeast cells, insect cells, or plant cells. In embodiments, the host cell is a bacterium, e.g., E. coli or lactic acid bacteria. The selection of an appropriate host is deemed to be within the scope of those skilled in the art. A recombinant host cell includes a host cell into which has been introduced a genetic construct.

In embodiments, a genetic construct is introduced into bacteria using a suitable technique. In embodiments, bacteria is transformed with a genetic construct by a suitable technique. Non-limiting examples of suitable techniques for introducing a nucleic acid into bacteria include conjugation, electroporation, transduction (e.g., injection of a nucleic acid by a bacteriophage), microinjection, by inducing competence (e.g., by addition of alkali cations, cesium, lithium, polyethylene glycol or by osmotic shock), or combinations thereof.

As would be appreciated by one of ordinary skill in the art, the genetic engineering strategies described herein to enhance GABA production in bacteria can also be applied to one or more of the following bacteria: probiotic bacteria; bacteria that are capable of producing lactic acid; and bacteria belonging to a genus including Bifidobacterium, Carnobacterium, Enterococcus, Lactobacillus, Lactococcus, Leuconostoc, Oenococcus, Pediococcus, Propionibacterium, Streptococcus, Tetragenococcus, Vagococcus, and Weissella.

Probiotic bacteria. In embodiments, probiotic bacteria include bacteria which when administered in adequate amounts confers a health benefit on the host. “Probiotic” or “probiotics” is used interchangeably with “probiotic bacteria”. Probiotic cultures are intended to assist the body's naturally occurring gut flora, an ecology of microbes, to re-establish themselves. Members of the lactic acid bacterial group are generally considered probiotic organisms. The best-known probiotics include Lactobacillus and Bifidobacterium.

In embodiments, probiotic bacteria have probiotic characteristics including tolerance to acidic and bile salt conditions, adhesion capability, antipathogenic activity, autoaggregation, and/or coaggregation abilities. Tolerance to acidic and bile salt conditions can be assessed as described by Dowarah et al. and Nami et al. (Dowarah et al. PLOS ONE, 2018, 13: e0192978; Nami et al. Front Microbiol., 2019, 10:300). Adhesion capability can be measured by an adhesion assay (Li et al. Front. Vet. Sci. 2020, 7, 49; Dowdell et al. Probiotics and antimicrobial proteins, 2020, 12 (2), 641-648). Autoaggregation and coaggregation can be measured by assays described by Collado et al. and Li et al. (Collado et al. Curr Microbiol., 2007, 55, 260-265; Li et al. Front. Vet. Sci. 2020, 7, 49). Antipathogenic activity can be measured by an Oxford cup assay (Muhammad et al. Pathogens, 2019, 8: E71; Li et al. Front. Vet. Sci. 2020, 7, 49). In embodiments, probiotic bacteria have safety properties including no or reduced hemolytic activity, resistance to multiple antibiotics, no or very few virulence factors, and possessing in vivo safety. Hemolytic activity can be assessed as described in Li et al. Front. Vet. Sci. 2020, 7, 49. Antibiotic resistance can be measured by, for example, a Kirby-Bauer disk diffusion test (Adetoye et al. BMC Microbiol., 2018, 18:96). Detection of virulence factors can be assessed by PCR amplification of genes encoding the virulence factors (Li et al. Front. Vet. Sci. 2020, 7, 49). In vivo safety of bacteria can be assessed by administering the bacteria orally to mice and assessing parameters of general health status of the mice, including body weight gain and organ index. For organ index, the spleen, liver, and kidney of the mice are collected and the weight of organ/body weight of the mice is determined (Li et al. Microb Cell Fact., 2019, 18:112).

In embodiments, probiotic bacteria include lactic acid bacteria (LAB). LAB produce lactic acid as the major metabolic end product of carbohydrate fermentation. The accumulation of lactic acid in the extracellular environment lowers the pH and inhibits the growth of spoilage agents. In embodiments, LAB include bacteria in the order Lactobacillies. In embodiments, LAB include bacteria in the phylum Firmicutes. In embodiments, LAB are gram-positive, low-guanine-cytosine content, acid-tolerant, non-sporulating, and non-respiring rod-shaped or spherical bacteria. In embodiments, LAB are found in decomposing plant and milk products.

In embodiments, LAB includes bacteria belonging to a genus selected from the group consisting of Bifidobacterium, Carnobacterium, Enterococcus, Lactobacillus, Lactococcus, Leuconostoc, Oenococcus, Pediococcus, Propionibacterium, Streptococcus, Tetragenococcus, Vagococcus, and Weissella.

In embodiments, examples of species of the genus Bifidobacterium include Bifidobacterium breve, Bifidobacterium longum, Bifidobacterium pseudolongum, Bifidobacterium animalis, Bifidobacterium adolescentis, Bifidobacterium angulatum, Bifidobacterium dentium, Bifidobacterium bifidum, Bifidobacterium lactis, Bifidobacterium catenulatum, Bifidobacterium pseudocatenulatum, and Bifidobacterium magnum.

In embodiments, examples of species of the genus Carnobacterium include Carnobacterium alterfunditum, Carnobacterium divergens, Carnobacterium funditium, Carnobacterium gallinarum, Carnobacterium iners, Carnobacterium inhibens, Carnobacterium jeotgali, Carnobacterium maltaromaticum, Carnobacterium mobile, Carnobacterium piscicola, Carnobacterium pleistocenium, and Carnobacterium viridans.

In embodiments, examples of species of the genus Enterococcus include Enterococcus alcedinis, Enterococcus aquimarinus, Enterococcus asini, Enterococcus avium, Enterococcus bulliens, Enterococcus burkinafasonensis, Enterococcus caccae, Enterococcus camelliae, Enterococcus canintestini, Enterococcus canis, Enterococcus casseliflavus, Enterococcus cecorum, Enterococcus columbae, Enterococcus crotali, Enterococcus devriesei, Enterococcus diestrammenae, Enterococcus dispar, Enterococcus durans, Enterococcus eurekensis, Enterococcus faecalis, Enterococcus faecium, Enterococcus gallinarum, Enterococcus gilvus, Enterococcus haemoperoxidus, Enterococcus hermanniensis, Enterococcus hirae, Enterococcus hulanensis, Enterococcus italicus, Enterococcus lactis, Enterococcus lemanii, Enterococcus malodoratus, Enterococcus massiliensis, Enterococcus mediterraneensis, Enterococcus moraviensis, Enterococcus mundtii, Enterococcus olivae, Enterococcus pallens, Enterococcus phoeniculicola, Enterococcus plantarum, Enterococcus pseudoavium, Enterococcus quebecensis, Enterococcus raffinosus, Enterococcus ratti, Enterococcus rivorum, Enterococcus rotai, Enterococcus saccharolyticus, Enterococcus saigonensis, Enterococcus silesiacus, Enterococcus sulfureus, Enterococcus solitarius, Enterococcus songbeiensis, Enterococcus termitis, Enterococcus thailandicus, Enterococcus ureasiticus, Enterococcus ureilyticus, Enterococcus viikkiensis, Enterococcus villorum, Enterococcus wangshanyuanii, Enterococcus xiangfangensis, and Enterococcus xinjiangensis.

In embodiments, examples of species of the genus Lactobacillus include Lactobacillus acidophilus, Lactobacillus crispatus, Lactobacillus amylovorus, Lactobacillus brevis, Lactobacillus buchneri, Lactobacillus casei, Lactobacillus curvatus, Lactobacillus delbrueckii subsp. bulgaricus, Lactobacillus farciminis, Lactobacillus fermentum, Lactobacillus futsaii, Lactobacillus gallinarum, Lactobacillus gasseri, Lactobacillus graminis, Lactobacillus helveticus, Lactobacillus johnsonii, Lactobacillus mali, Lactobacillus mucosae, Lactobacillus namurensis, Lactobacillus otakiensis, Lactobacillus paracasei, Lactobacillus paralimentarius, Lactobacillus plantarum, Lactobacillus reuteri, Lactobacillus rhamnosus, Lactobacillus rossiae, Lactobacillus sakei, Lactobacillus salivarius, Lactobacillus viridescens, and Lactobacillus zeae.

In embodiments, examples of species of the genus Lactococcus include Lactococcus chungangensis, Lactococcus cremoris, Lactococcus formosensis, Lactococcus fujiensis, Lactococcus garvieae (including subsp. garvieae and subsp. Bovis), Lactococcus hircilactis, Lactococcus lactis (including subsp. cremoris, subsp. hordniae, subsp. lactis, and subsp. tructae), Lactococcus laudensis, Lactococcus nasutitermitis, Lactococcus petauri, Lactococcus piscium, Lactococcus plantarum, Lactococcus raffinolactis, and Lactococcus taiwanensis.

In embodiments, probiotic bacteria include Lactococcus lactis. L. lactis is a Gram-positive bacterium used in the production of food products such as buttermilk, cheese, pickled vegetables, beer or wine, breads, and soymilk kefir, due to the bacterium's ability to produce lactic acid, which can be used to aid in food fermentation. Genetically modified L. lactis has also been used for the treatment of human disease (Braat et al. Clinical Gastroenterology and Hepatology 2006, 4 (6), P754-759). L. lactis cells appear ovoid with a typical length of 0.5-1.5 μm, and cells group in pairs and short chains. L. lactis does not produce spores and are not motile. Because of its use in the food industry, L. lactis has generally recognized as safe (GRAS) status. L. lactis can be isolated from the dairy environment or from plant material.

In embodiments, examples of species of the genus Leuconostoc include Leuconostoc carnosum, Leuconostoc citreum, Leuconostoc falkenbergense, Leuconostoc fallax, Leuconostoc garlicum, Leuconostoc gelidum, Leuconostoc holzapfelii, Leuconostoc inhae, Leuconostoc kimchii, Leuconostoc lactis, Leuconostoc kitchii, Leuconostoc mesenteroides, Leuconostoc miyukkimchii, Leuconostoc palmae, Leuconostoc pseudomesenteroides, Leuconostoc rapi, and Leuconostoc suionicum.

In embodiments, examples of species of the genus Oenococcus include Oenococcus alcoholitolerans, Oenococcus kitaharae, Oenococcus oeni, and Oenococcus sicerae.

In embodiments, examples of species of the genus Pediococcus include Pediococcus acidilactici, Pediococcus argentinicus, Pediococcus cellicola, Pediococcus claussenii, Pediococcus damnosus, Pediococcus ethanolidurans, Pediococcus inopinatus, Pediococcus parvulus, Pediococcus pentosaceus, Pediococcus perniciosus, Pediococcus siamensis, and Pediococcus stilesii.

In embodiments, examples of species of the genus Propionibacterium include Propionibacterium freudenreichii.

In embodiments, examples of species of the genus Streptococcus include Streptococcus acidominimus, Streptococcus agalactiae, Streptococcus alactolyticus, Streptococcus anginosus, Streptococcus australis, Streptococcus caballi, Streptococcus cameli, Streptococcus canis, Streptococcus caprae, Streptococcus castoreus, Streptococcus cricetid, Streptococcus constellatus, Streptococcus cristatus, Streptococcus cuniculi, Streptococcus danieliae, Streptococcus dentasini, Streptococcus dentiloxodontae, Streptococcus dentirousetti, Streptococcus devriesei, Streptococcus didelphis, Streptococcus downei, Streptococcus dysgalactiae, Streptococcus entericus, Streptococcus equi, Streptococcus equinus, Streptococcus ferus, Streptococcus gallinaceus, Streptococcus gallolyticus, Streptococcus gordonii, Streptococcus halichoeri, Streptococcus halotolerans, Streptococcus henryi, Streptococcus himalayensis, Streptococcus hongkongensis, Streptococcus hyointestinalis, Streptococcus hyovaginalis, Streptococcus ictalurid, Streptococcus infantarius, Streptococcus infantis, Streptococcus iniae, Streptococcus intermedius, Streptococcus lactarius, Streptococcus loxodontisalivarius, Streptococcus lutetiensis, Streptococcus macacae, Streptococcus marimammalium, Streptococcus marmotae, Streptococcus massiliensis, Streptococcus merionis, Streptococcus minor, Streptococcus mitis, Streptococcus moroccensis, Streptococcus mutans, Streptococcus oralis, Streptococcus oricebi, Streptococcus oriloxodontae, Streptococcus orisasini, Streptococcus orisratti, Streptococcus orisuis, Streptococcus ovis, Streptococcus panodentis, Streptococcus pantholopis, Streptococcus parasanguinis, Streptococcus parasuis, Streptococcus parauberis, Streptococcus peroris, Streptococcus pharynges, Streptococcus phocae, Streptococcus pluranimalium, Streptococcus plurextorum, Streptococcus pneumoniae, Streptococcus porci, Streptococcus porcinus, Streptococcus porcorum, Streptococcus pseudopneumoniae, Streptococcus pseudoporcinus, Streptococcus pyogenes, Streptococcus ratti, Streptococcus rifensis, Streptococcus rubneri, Streptococcus rupicaprae, Streptococcus salivarius (include subsp. thermophilus), Streptococcus saliviloxodontae, Streptococcus sanguinis, Streptococcus sinensis, Streptococcus sobrinus, Streptococcus suis, Streptococcus tangierensis, Streptococcus thoraltensis, Streptococcus troglodytae, Streptococcus troglodytidis, Streptococcus tigurinus, Streptococcus thermophilus, Streptococcus uberis, Streptococcus urinalis, Streptococcus ursoris, Streptococcus vestibularis, and Streptococcus zooepidemicus.

In embodiments, examples of species of the genus Tetragenococcus include Tetragenococcus halophilus, Tetragenococcus koreensis, Tetragenococcus muriaticus, Tetragenococcus osmophilus, and Tetragenococcus solitarius.

In embodiments, examples of species of the genus Vagococcus include Vagococcus acidifermentans, Vagococcus bubulae, Vagococcus carniphilus, Vagococcus coleopterorum, Vagococcus elongatus, Vagococcus entomophilus, Vagococcus fessus, Vagococcus fluvialis, Vagococcus humatus, Vagococcus hydrophili, Vagococcus lutrae, Vagococcus martis, Vagococcus penaei, Vagococcus salmoninarum, Vagococcus silage, Vagococcus teuberi, Vagococcus vulneris, Vagococcus xieshaowenii, and Vagococcus zengguangii.

In embodiments, examples of species of the genus Weissella include Weissella cibaria, Weissella confusa, Weissella halotolerans, Weissella hellenica, Weissella kandleri, Weissella kimchii, Weissella koreensis, Weissella minor, Weissella paramesenteroides, Weissella soli, Weissella thailandensis, and Weissella viridescens.

Probiotic bacteria genetically modified as described herein can be propagated under conditions and in media known to one of skill in the art.

In embodiments, genetically modified probiotic bacteria can be prepared via culture under adequate conditions using a medium conventionally used for culture of probiotic bacteria. A natural medium or a synthetic medium can be used as a culture medium as long as it contains a carbon source, a nitrogen source, a mineral salt, an agent to select for the genetic construct (e.g., erythromycin), and other components, and it enables culture of genetically modified probiotic bacteria with efficiency. Those skilled in the art can adequately select a known medium appropriate for a bacterial strain to be used. Examples of a carbon source that can be used include lactose, glucose, sucrose, fructose, galactose, and blackstrap molasses. Examples of a nitrogen source that can be used include organic nitrogen-containing substances such as casein hydrolysate, whey protein hydrolysate, and soy protein hydrolysate. Examples of a mineral salt that can be used include phosphate, sodium, potassium, and magnesium. Examples of an appropriate medium for culture of probiotic bacteria include an MRS liquid medium, a GAM medium, a BL medium, Briggs Liver Broth, animal milk, skim milk, and milk-derived whey. Examples of a natural medium that can be used include tomato juice, carrot juice, other vegetable juice, apple juice, pineapple juice, and grape juice.

In addition, culture of genetically modified probiotic bacteria of the disclosure can be performed at 20° C. to 50° C., or 25° C. to 42° C., at 30° C., or at 37° C. In embodiments, culture of genetically modified probiotic bacteria of the disclosure can be under anaerobic conditions. Temperature conditions can be adjusted using a thermostatic bath, a mantle heater, a jacket, or the like. In addition, the term “anaerobic conditions” used herein refers to a low-oxygen environment in which probiotic bacteria can proliferate. For instance, in such environment, anaerobic conditions can be provided by using an anaerobic chamber, an anaerobic box, an airtight container or bag containing a deoxidizer, or the like, or by simply sealing a culture container in an airtight manner. The format of culture includes static culture, shake culture, and tank culture. In addition, the period of culture can be determined to be 3 hours to 96 hours. In embodiments, the pH of the medium can be maintained at 4.0 to 8.0 at the beginning of culture.

In embodiments, genetically modified probiotic bacteria producing GABA can be cultured and harvested at any stage of the culture, including pre-logarithmic, logarithmic, and stationary phase. In embodiments, genetically modified probiotic bacteria producing GABA can be cultured to an optical density (OD) 600 nm of 0.5 to 2. In embodiments, genetically modified probiotic bacteria producing GABA can be cultured to an OD 600 nm of 0.5, 0.6, 0.7, 0.8, 0.9, 1.0, 1.1, 1.2, 1.3, 1.4, 1.5, 1.6, 1.7, 1.8, 1.9, 2.0, or greater. In embodiments, genetically modified probiotic bacteria producing GABA can be cultured and harvested at OD 600 nm of 0.5 to 2. In embodiments, genetically modified probiotic bacteria producing GABA can be cultured and harvested at OD 600 nm of 0.5, 0.6, 0.7, 0.8, 0.9, 1.0, 1.1, 1.2, 1.3, 1.4, 1.5, 1.6, 1.7, 1.8, 1.9, 2.0, or greater.

In embodiments, L. lactis can be grown at 30° C. without shaking in M17 broth or on M17 agar plates (Difco Laboratories, Detroit, MI) supplemented with a carbon source (e.g. 0.5% glucose). M17 broth includes per 950 mL: 5.0 g pancreatic digest of casein, 5.0 g soy peptone, 5.0 g beef extract, 2.5 g yeast extract, 0.5 g ascorbic acid, 0.25 g magnesium sulfate, and 19.0 g disodium-β-glycerophosphate. M17 media supplemented with 0.5% glucose is known as GM17. To select for L. lactis containing a genetic construct with an erythromycin resistance gene (erm), GM17 media containing 5 μg/mL erythromycin (ERM GM17) can be used to select for bacteria that retain the genetic construct. In embodiments, L. lactis can be grown to an OD 600 nm of 0.5, 0.6, 0.7, 0.8, 0.9, 1.0, 1.1, 1.2, 1.3, 1.4, 1.5, 1.6, 1.7, 1.8, 1.9, 2.0, or greater. Genetically modified probiotic bacteria can be stored long term in GM17 media supplemented with 15% glycerol and frozen at −80° C.

In embodiments, genetically modified probiotic bacteria producing GABA can produce greater amounts of GABA when glutamic acid/glutamate is provided in culture as compared to genetically modified probiotic bacteria producing GABA cultured in media not supplemented with glutamic acid/glutamate. In embodiments, the glutamic acid/glutamate is provided in culture as glutamic acid HCl. In embodiments, the glutamic acid/glutamate (e.g., glutamic acid HCl) is provided in culture at a concentration of 50 mM or more. In embodiments, the glutamic acid/glutamate (e.g., glutamic acid HCl) is provided in culture at a concentration of 50 mM, 100 mM, 150 mM, or 200 mM.

In embodiments, the obtained culture of genetically modified probiotic bacteria producing GABA can be directly used. In embodiments, the culture of genetically modified probiotic bacteria can be further subjected to treatments including sterilization, crude purification via centrifugation, and/or solid-liquid separation via filtration, according to need. In addition, genetically modified probiotic bacteria of the present disclosure can be in the form of viable bacterial cells or dead bacterial cells and/or in the form of wet bacterial cells or dried bacterial cells.

In embodiments, a sterilized product can be prepared by sterilization treatment of the genetically modified probiotic bacteria. Sterilization treatment can include filtration sterilization, radiation disinfection, superheat disinfection, and pressure disinfection.

In embodiments, a heated product can be prepared by heat treatment of the genetically modified probiotic bacteria. Heat treatment can include high temperature treatment (e.g., 80° C. to 150° C.) of the genetically modified probiotic bacteria for a period of time (e.g., 10 minutes to 1 hour, or 10 to 20 minutes).

In embodiments, a disrupted product or a cell-free extract can be prepared by disrupting, fracturing, comminution, size reduction, crushing, pulverization, disintegration, or grinding the genetically modified probiotic bacteria. For example, physical disruption (e.g., agitation or filter filtration), enzymatic lysis treatment, chemical treatment, and/or autolysis induction treatment can be performed.

In embodiments, an extract can be obtained via extraction of the genetically modified probiotic bacteria with an adequate aqueous or organic solvent. For example, the genetically modified probiotic bacteria can be immersed in an aqueous or organic solvent (e.g., water, methanol, or ethanol), or can be agitated or refluxed in the solvent.

In embodiments, the genetically modified probiotic bacteria can be processed into a powdery product (powder) or granular product via drying. Drying methods include spray drying, drum drying, vacuum drying, and lyophilization, which can be used alone or in combination.

In embodiments, GABA can be purified from the genetically modified probiotic bacteria by a known separation/purification method. Examples of such separation/purification methods include: a method involving salt precipitation, or organic solvent precipitation in accordance with degrees of solubility; a method involving dialysis, ultrafiltration or gel filtration in accordance with molecular weight differences; a method involving ion-exchange chromatography in accordance with charge differences; a method involving affinity chromatography in accordance with degrees of specific binding; and a method involving hydrophobic chromatography, or reverse phase chromatography in accordance with degrees of hydrophobicity, or a combination thereof.

The genetically modified probiotic bacteria can include bacterial cells of a single species or genus of probiotic bacteria. Alternatively, genetically modified probiotic bacteria can include bacterial cells of two or more species or genus of probiotic bacteria. In embodiments, the genetically modified probiotic bacteria can contain a combination of genetically modified probiotic bacteria treated in different ways (i.e. a population that has been sterilized and a population that has been heat treated).

In embodiments, genetically modified probiotic bacteria can be further formulated for oral consumption as described herein.

Compositions. The present disclosure provides compositions and formulations including GABA-producing genetically modified probiotic bacteria. In embodiments, the probiotic bacteria is L. lactis. In embodiments, the compositions and formulations including GABA-producing genetically modified probiotic bacteria are in a form including lyophilizates, powders, granules, tablets, soft-gel capsules, and suspensions. In embodiments, the compositions and formulations including GABA-producing genetically modified probiotic bacteria can be prepared as a lyophilized powder. In embodiments, the lyophilized powder can be dispersed in water, juices, or any other liquids.

Suspensions of GABA-producing genetically modified probiotic bacteria can be prepared by suspending or diluting the bacterial cells in an adequate solvent. Examples of a solvent that can be used include water, physiological saline, and phosphate buffer saline (PBS).

The compositions and formulations of the present disclosure can be pharmaceutical, dietetic, nutritional, or nutraceutical compositions. Nutraceuticals include products derived from food sources that provide extra health benefits in addition to the basic nutritional value found in foods. In embodiments, nutraceuticals can be grouped into categories that include dietary supplements, functional food, medicinal food, and farmaceuticals. A dietary supplement includes a product that contains nutrients derived from food products and is often concentrated in liquid, capsule, powder, or pill form. Dietary supplements are regulated by the United States Food and Drug Administration (FDA). Functional food includes whole foods and enhanced dietary components that can reduce the risk of chronic disease and provide a health benefit beyond the traditional nutrients it contains. Medical food is formulated to be consumed or administered internally, under the supervision of a qualified physician, to manage a disease or condition for which distinctive nutritional requirements are established. Farmaceuticals include medically valuable components produced from modified agricultural crops or animals.

In embodiments, a composition including GABA-producing genetically modified probiotic bacteria can be mixed with minerals or vitamin supplements. In embodiments, minerals can include macrominerals and trace minerals. In embodiments, minerals can include: calcium, chloride, magnesium, phosphorus, potassium, sodium, sulfur, cobalt, copper, fluoride, iodine, iron, manganese, selenium, zinc, or a combination thereof. In embodiments, vitamins can include vitamin A, thiamine (vitamin B1), riboflavin (vitamin B2), niacin (vitamin B3), pantothenic acid (vitamin B5), biotin, vitamin B6, vitamin B12, folate, vitamin C (ascorbic acid), vitamin D, vitamin E, vitamin K, choline, carnitine, or a combination thereof.

In embodiments, the compositions and formulations including GABA-producing genetically modified probiotic bacteria can be incorporated in food such as dairy products (e.g., yogurt, milk, cheese, kefir, ice cream, butter).

The compositions and formulations of the present disclosure can include a prebiotic. Prebiotics include compounds that selectively stimulate the growth or activity of desirable microorganisms. In embodiments, prebiotics include a complex carbohydrate such as fiber. In embodiments, prebiotics include dietary fibers including soy fiber, sugarbeet fiber, pea fiber, corn bran, and oat fiber. In embodiments, prebiotics include components from fruits and vegetables.

Examples of prebiotics include chicory root, dandelion greens, Jerusalem artichoke, garlic, onions, leeks, asparagus, bananas, barley, oats, apples, konjac root, cocoa, burdock root, flaxseeds, jacon root, jicama root, wheat bran, and seaweed. In embodiments, prebiotics include amino acids (e.g., arginine, glutamate, ornithine). In embodiments, prebiotics include oligosaccharides. In embodiments, prebiotics include: fructo-oligosaccharides (FOS); galacto-oligosaccharides; xylo-oligosaccharides; hemicelluloses (e.g., arabinoxylan, xylan, xyloglucan, glucomannan); inulin, chitin; lactulose; mannan oligosaccharides; oligofructose-enriched inulin; gums (e.g., guar gum, gum arabic, carregenaan); oligofructose, oligodextrose; tagatose; resistant maltodextrins (e.g., resistant starch); trans-galacto-oligosaccharide; and pectins (e.g., xylogalactouronan, citrus pectin, apple pectin, rhamnogalacturonan-1). In embodiments, prebiotics include polyamines (e.g., spermidine, putrescine).

In embodiments, the compositions and formulations of the present disclosure can include an enteric coating or similar to survive the acidity of the stomach and enabled delivery into the small or large intestines.

Exemplary excipient classes for oral compositions and formulations include binders, buffers, chelators, coating agents, colorants, complexation agents, diluents (i.e., fillers), disintegrants, emulsifiers, flavoring agents, glidants, lubricants, preservatives, releasing agents, surfactants, stabilizing agents, solubilizing agents, sweeteners, thickening agents, wetting agents, and vehicles.

Binders are substances used to cause adhesion of powder particles in granulations. Exemplary binders include acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, and polyethylene glycol.

Colorants can be included in the oral compositions to impart color. Exemplary colorants include grape skin extract, beet red powder, beta carotene, annato, carmine, turmeric, and paprika. Additional colorants include FD&C Red No. 3, FD&C Red No. 20, FD&C Yellow No. 6, FD&C Blue No. 2, D&C Green No. 5, FD&C Orange No. 5, D&C Red No. 8, caramel, and ferric oxide.

Diluents can enhance the granulation of oral compositions. Exemplary diluents include microcrystalline cellulose, sucrose, dicalcium phosphate, starches, lactose and polyols of less than 13 carbon atoms, such as mannitol, xylitol, sorbitol, maltitol and pharmaceutically acceptable amino acids, such as glycin.

Disintegrants also can be included in the oral compositions in order to facilitate dissolution. Disintegrants, including permeabilizing and wicking agents, are capable of drawing water or saliva up into the oral compositions which promotes dissolution from the inside as well as the outside of the oral compositions. Such disintegrants, permeabilizing and/or wicking agents that can be used include: starches, such as corn starch, potato starch, pre-gelatinized and modified starches thereof; cellulosic agents, such as Ac-di-sol, microcrystalline cellulose, croscarmellose sodium, hydroxymethylcellulose, hydroxypropylcellulose, and hydroxyopropylmethylcellulose; montmorrilonite clays; cross-linked PVP; sweeteners; bentonite; alginates; sodium starch glycolate; gums, such as agar, guar, locust bean, karaya, pectin, Arabic, xanthan and tragacanth; silica with a high affinity for aqueous solvents, such as colloidal silica and precipitated silica; polysaccharides such as maltodextrins and beta-cyclodextrins; and polymers, such as carbopol. Dissolution of the oral compositions can be facilitated by including relatively small particles sizes of the ingredients used.

Exemplary dispersing or suspending agents include acacia, alginate, dextran, fragacanth, gelatin, hydrogenated edible fats, methylcellulose, polyvinylpyrrolidone, sodium carboxymethyl cellulose, sorbitol syrup, and synthetic natural gums.

Emulsifiers are surface active substances which promote the suspension of one liquid in another and promote the formation of a stable mixture, or emulsion, of oil and water. Exemplary emulsifiers include acacia, anionic emulsifying wax, calcium stearate, carbomers, ceto stearyl alcohol, cetyl alcohol, cholesterol, diethanolamine, ethylene glycol palmitostearate, glycerin monostearate, glyceryl monooleate, hydroxypropyl cellulose, hypromellose, lanolin hydrous, lanolin alcohols, lecithin, medium-chain triglycerides, methylcellulose, mineral oil, monobasic sodium phosphate, monoethanolamine, nonionic emulsifying wax, oleic acid, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum, and combinations thereof.

Flavorants are natural or artificial compounds used to impart a pleasant flavor and often odor to oral compositions. Exemplary flavorants include natural and synthetic flavor oils, flavoring aromatics, extracts from plants, leaves, flowers, and fruits and combinations thereof. Such flavorants include anise oil, cinnamon oil, vanilla, vanillin, cocoa, chocolate, natural chocolate flavor, menthol, grape, peppermint oil, oil of wintergreen, clove oil, bay oil, anise oil, eucalyptus, thyme oil, cedar leave oil, oil of nutmeg, oil of sage, oil of bitter almonds, cassia oil; citrus oils, such as lemon, orange, lime and grapefruit oils; and fruit essences, including apple, pear, peach, berry, wildberry, date, blueberry, kiwi, strawberry, raspberry, cherry, plum, pineapple, and apricot. In particular embodiments, flavorants that can be used include natural berry extracts and natural mixed berry flavor, as well as citric and malic acid.

Glidants improve the flow of powder blends during manufacturing and minimize oral composition weight variation. Exemplary glidants include silicon dioxide, colloidal or fumed silica, magnesium stearate, calcium stearate, stearic acid, cornstarch, and talc.

Lubricants are substances used in oral compositions that reduce friction during composition compression. Exemplary lubricants include stearic acid, calcium stearate, magnesium stearate, zinc stearate, talc, mineral and vegetable oils, benzoic acid, poly(ethylene glycol), glyceryl behenate, stearyl fumarate, and sodium lauryl sulfate.

Preservatives are substances used in compositions to prevent the growth of microorganisms and/or to prevent degradation of the active ingredient. Exemplary preservatives include parabens, chlorobutanol, phenol, thimerosal, methyl p-hydroxybenzoates, propyl p-hydroxybenzoates, and sorbic acid. In embodiments, the compositions and formulations of the present disclosure can include an effective amount of an anti-fungal agent, an anti-viral agent, an anti-parasitic agent, or a combination thereof.

Exemplary sweeteners include aspartame, dextrose, fructose, high fructose corn syrup, maltodextrin, monoammonium glycyrrhizinate, neohesperidin dihydrochalcone, potassium acesulfame, saccharin sodium, stevia, sucralose, and sucrose.

The compositions and formulations of the present disclosure can include additives. Examples of additives include taurine, glutathione, carnitine, creatine, coenzyme Q, glucuronic acid, glucuronolactone, Capsicum extract, ginger extract, cacao extract, guarana extract, garcinia extract, theanine, capsaicin, capsiate, organic acids, flavonoids, polyphenols, catechins, and xanthine derivatives.

Embodiments include swallowable compositions. Swallowable compositions are those that do not readily dissolve when placed in the mouth and can be swallowed whole without chewing or discomfort. U.S. Pat. Nos. 5,215,754 and 4,374,082 describe methods for preparing swallowable compositions. In particular embodiments, swallowable compositions can have a shape containing no sharp edges and a smooth, uniform and substantially bubble free outer coating.

To prepare swallowable compositions, GAD-L. lactis bacteria and other ingredients can be combined in an intimate admixture with a suitable carrier according to conventional compounding techniques. In embodiments of the swallowable compositions, the surface of the compositions can be coated with a polymeric film. Such a film coating has several beneficial effects. First, it reduces the adhesion of the compositions to the inner surface of the mouth, thereby increasing the subject's ability to swallow the compositions. Second, the film can aid in masking the unpleasant taste of certain ingredients. Third, the film coating can protect the compositions from atmospheric degradation. Polymeric films that can be used in preparing the swallowable compositions include vinyl polymers such as polyvinylpyrrolidone, polyvinyl alcohol and acetate, cellulosics such as methyl and ethyl cellulose, hydroxyethyl cellulose and hydroxylpropyl methylcellulose, acrylates and methacrylates, copolymers such as the vinyl-maleic acid and styrene-maleic acid types, and natural gums and resins such as zein, gelatin, shellac and acacia.

In addition to those described above, any appropriate fillers and excipients can be utilized in preparing the swallowable or any other oral composition described herein so long as they are consistent with the described objectives. Excipients are commercially available from companies such as Aldrich Chemical Co., FMC Corp, Bayer, BASF, Alexi Fres, Witco, Mallinckrodt, Rhodia, ISP, and others.

Oral compositions can be individually wrapped or packaged as multiple units in one or more packages, cans, vials, blister packs, or bottles of any size. Doses are sized to provide therapeutically effective amounts.

Additional information can be found in WADE & WALLER, HANDBOOK OF PHARMACEUTICAL EXCIPIENTS (2nd ed. 1994) and Remington's Pharmaceutical Sciences, 18th Ed. Mack Printing Company, 1990. Moreover, compositions or formulations can be prepared to meet sterility, pyrogenicity, general safety, and purity standards as required by U.S. FDA and/or other relevant foreign regulatory agencies.

Methods of Use. Compositions disclosed herein can be used to treat subjects (humans, veterinary animals (dogs, cats, reptiles, birds, etc.), livestock (horses, cattle, goats, pigs, chickens, etc.), and research animals (monkeys, rats, mice, fish, etc.)). Treating subjects includes providing therapeutically effective amounts. Therapeutically effective amounts include those that provide effective amounts, prophylactic treatments, and/or therapeutic treatments. A subject in need of treatment includes a subject having symptoms associated with and/or afflicted by a neurological disease, inflammatory disease, and/or behavioral and mood disorders described herein.

Two types of GABA receptors, GABA_{A} receptor (GABA_{A}R) and GABA_{B} receptor (GABA_{B}R), are activated when GABA binds. The GABA_{A}R is a ligand gated chloride ion (Cl—) channel found on the postsynaptic surface of neurons. Being a pentameric transmembrane receptor, GABA_{A}R is large. The GABA_{B}R is a metabotropic G protein binding receptor located on both pre- and postsynaptic membranes. Both types of receptors are widespread in the CNS. In addition to being expressed in cells of the neuronal system, GABAAR is also expressed in immune cells. Importantly, targeting GABAAR promotes significant functional changes in immune cells. For example, activation of GABAAR on T cells inhibits antigen specific T cell proliferation and decreases pro-inflammatory T cell responses. As T cells play a major role in MS, it may be possible to regulate the level of GABA to control MS severity.

The gut-brain axis refers to the bi-directional biochemical signaling that occurs between the gastrointestinal tract and the CNS. The vagus nerve is the main nerve of the parasympathetic branch of the autonomic nervous system (ANS) and regulates important bodily functions such as mood, immune responses, digestion, and heart rate. It establishes a connection between the gastrointestinal tract and the brain. Activation of GABAAR in the gut could affect vagus nerve function to modulate effects in the brain. For example, stimulation of the vagus nerve can increase production of GABA in the brain.

On the other hand, glutamate, the substrate that GAD uses to produce GABA is the primary fast excitatory neurotransmitter that functions in learning, memory, long-term potentiation, and synaptic plasticity. Excessive glutamate is implicated in neuronal cell death and neurodegenerative diseases such as Alzheimer's, Parkinson's disease, and MS.

In embodiments, the present disclosure provides for delivering a therapeutic composition of genetically modified probiotic bacteria that has enhanced GABA production to the intestinal tract to consistently deliver GABA into systemic circulation (e.g., into the nervous system).

In embodiments, GAD-L. lactis can be prepared as a lyophilized powder and administered orally either plain, incorporated in food such as yogurts, dairy products, or mixed with other mineral or vitamin supplements. Alternatively, the lyophilized powder can be dispersed in water, juices, or any other liquids.

GABA's effects on a number of conditions have been investigated. For example, GABA has been implicated in prevention of sleeplessness and depression and in alleviating or preventing hypertension, diabetes, cancer, inflammation, and allergy. GABA may protect the liver, the kidneys, and the intestine (Ngo and Vo. Molecules. 2019, 24 (15), 2678).

In embodiments, compositions or formulations including genetically modified GAD-L. lactis of the disclosure can be used to treat rare diseases caused by genetic disorders of the GABA metabolic pathway, including GABA-transaminase deficiency (GABA-T deficiency), succinic semialdehyde dehydrogenase (SSADH) deficiency, and homocarnosinosis, which are all diseases that involve the GABA catabolic pathway. Nonsyndromic cleft lip with or without cleft palate has been linked with specific glutamate decarboxylase (GAD67) haplotypes and may represent a disorder of GABA synthesis.

GABA-T deficiency is a rare autosomal recessive disorder characterized by seizures, abnormal development, and high levels of GABA in serum and cerebrospinal fluid (CSF). In GABA-T deficiency, GABA transaminase, an enzyme that metabolizes GABA to succinic semialdehyde, is deficient. Clinical manifestations of GABA-T deficiency include neonatal seizures, lethargy, decreased muscle tone, overactive reflexes, severely slowed psychomotor development, poor feeding, and high-pitched cries. Electroencephalograms (EEGs) and computed tomography (CT) can be used to assess brain function and anatomy. GABA-T deficiency can be diagnosed by: elevated levels of GABA and beta-alanine in CSF and/or plasma; aminoaciduria; and/or GABA-T activity in liver, lymphocytes from whole blood, and/or Epstein-Barr virus-transformed cultured lymphocytes.

SSADH deficiency is an autosomal recessive disorder where a deficiency of the enzyme leads to accumulation of γ-hydroxybutyrate (GHB) because GABA cannot be converted to succinic acid. SSADH deficiency is characterized by excessive levels of GHB in fluids, such as plasma, urine, and CSF. Subjects with SSADH deficiency can have neuropsychiatric problems, developmental delays, language impairment, seizures, decreased muscle tone, muscles less responsive to stimuli, and ataxia. Abnormalities in the brain such as cerebral and cerebellar atrophy can be detected by magnetic resonance imaging (MRI) and positron emission tomography. 3H-MRI and magnetic resonance scanning (MRS) can be used to assess neuronal markers (e.g., N-acetylasparatate, choline, creatine) and GABA. SSADH deficiency can be diagnosed by GHB levels in urine by gas chromatography/mass spectrometry. Therapies for this disease include Vigabatrin, an irreversible inhibitor of GABA-T, which is provided as an antiseizure medication. Seizures are also treated with carbamazepine and lamotrigine. Therapeutics for anxiety and behavioral problems include benzodiazepines, risperidal, fluoxetine, and methylphenidate.

Homocarnosinosis is considered to be a form of carnosinase deficiency. Serum carnosinase breaks down homocarnosine, a brain-specific dipeptide of GABA and histidine, while a carnosine synthetase synthesizes the dipeptide. The disease is characterized by progressive mental deterioration and retinal pigmentation. Subjects afflicted with homocarnosinosis can have elevated homocarnosine levels in CSF.

In embodiments, immune cells expressing receptors for GABA can respond to the presence of the neurotransmitter, altering their phenotype and/or function, resulting in tolerogenic or anti-inflammatory effects that might be protective against diseases with an inflammatory component. The diseases include conditions of the central nervous system, but also diseases targeting other tissues or organs, such as the gastrointestinal tract, as described herein. In embodiments, the GABA receptors are GABAAR. In embodiments, compositions or formulations including genetically modified GAD-L. lactis of the disclosure can be used to reduce or prevent symptoms associated with inflammatory diseases described herein. In embodiments, compositions or formulations including genetically modified GAD-L. lactis of the disclosure can be used to treat inflammatory diseases or disorders. In embodiments, inflammatory diseases or disorders include: MS; cachexia (e.g., secondary to chronic inflammation as seen in cancer); inflammatory bowel diseases (IBD; Crohn's disease, ulcerative colitis); psoriatic arthritis; rheumatoid arthritis (RA); Type 1 diabetes; Type 2 Diabetes; Sjögren's syndrome; systemic lupus erythematosus (lupus or SLE); celiac disease; Graves' disease; Hashimoto's thyroiditis; Addison's disease; dermatomyositis; psoriasis; chronic inflammatory demyelinating polyneuropathy (CIDP); Guillain-Barre syndrome; myasthenia gravis; and vasculitis.

MS is the most common reason for non-traumatic disability in adults from developed nations, affecting millions of people. MS is a neurodegenerative disease characterized by inflammatory lesions of the CNS that lead to demyelination of neurons. The inflammatory lesions are caused by immune cells infiltrating the CNS and attacking the myelin sheath. The myelin sheath is the insulating layer around nerves; the sheath's destruction can lead to development of sclerosed plaques. As the damage increases, neuronal signaling is disrupted, leading to neurological symptoms such as vision problems, depression, fatigue, dizziness, and paralysis. Ultimately, the disease can lead to death. Forms of MS include relapsing/remitting MS (RRMS), secondary progressive MS (SPMS), and primary progressive MS (PPMS). RRMS is the most common form. RRMS is characterized by periods of remission followed by relapses where neurological symptoms worsen. About 85% of newly diagnosed MS patients are diagnosed with RRMS. In 10-15 years around 50% of those patients will develop SPMS, where neurological symptoms gradually worsen with few if any remissions. Around 15% of newly diagnosed patients are diagnosed with PPMS where neurological symptoms gradually worsen with few remissions from the start. MRI scans of the brain at multiple locations and over time may help to diagnose MS. There is no cure for MS. Treatment can include: anti-inflammatory cytokines to decrease inflammation in the CNS (e.g., type I interferon beta-1a and 1b); biologics (e.g., antibodies that target particular immune cells; alemtuzumab, ocrelizumab, natalizumab); hematopoietic stem cell transplantation, where a patient's immune cells are eliminated/reduced and replaced with bone marrow hematopoietic stem cells.

One of the most common animal models for MS is experimental autoimmune encephalomyelitis (EAE). This model has been used to study MS for over 100 years. Due to its extensive use and ease, it works well for comparing treatments. EAE induction can be either active or passive. In passive EAE induction, encephalitogenic T cells are isolated from the lymphoid tissues of animals already immunized with myelin antigen. These T cells are specific to myelin antigen and when transferred to naive recipients attack the myelin sheath and start the disease. In active EAE induction, T-cell mediated immunity is induced by injection of myelin antigens (e.g., myelin oligodendrocyte glycoprotein peptide 35-55, or MOG35-55; proteolipid protein (PLP); myelin basic protein (MBP); peptides corresponding to the encephalitogenic portions of these proteins). Induction requires the emulsification of one of the self-antigens in complete Freund's adjuvant and additional intraperitoneal or intravenous administration of pertussis toxin. Induction can be performed using a commercial EAE induction kit (Hooke labs, kit EK-2110). In either passive or active EAE induction, antigen presenting cells activate CD4+ T cells by presenting myelin peptides, and myelin-specific CD4+ T cells cross the blood brain barrier into the CNS parenchyma. Activation of T cells lead to a pro-inflammatory cascade mediated by cytokines. Continuous inflammation results in myelin sheath damage and eventual paralysis. Spontaneous EAE can also be induced, which requires special transgenic animals (Gold et al., Brain, 2006, 129, 1953-1971; Burrows et al., Mult Scler, 2019, 25, 306-324). Active EAE induced in C57BL/6 mice (B6 mice) was the animal model selected for experiments in the Examples. A protocol for the induction and monitorization of the disease has been described (Stromnes and Goverman. Nature Protocols 2006, 1, 1810-1819). EAE mice are scored as described previously (Stromnes and Goverman. Nature Protocols 2006, 1, 1810-1819): 0—no detectable signs of EAE, 0.5—distal limp tail, 1.0—complete limp tail, 1.5—limp tail and hind limb weakness, 2.0—unilateral partial hind limb paralysis, 2.5—bilateral partial hind limb paralysis, 3.0—complete bilateral hind limb paralysis, 3.5—complete bilateral hind limb paralysis and partial front limb paralysis, 4.0—quadriplegia. Those mice that show clinical scores>3.0 for two consecutive days are euthanized due to humane reasons and per IACUC guidelines.

In embodiments, compositions or formulations including genetically modified GAD-L. lactis of the disclosure can be used to treat GABA-deficiency conditions, conditions in which GABA receptor mediated signaling is impaired, and/or conditions with an excess of excitatory neurotransmitters (such as glutamate) including: alcoholism, anxiety, autism, MS, schizophrenia, Parkinson's Disease, Huntington's disease, epilepsy, post-traumatic stress disorder (PTSD), and stroke and its complications.

GABA_{A}R undergo allosteric modulation by ethanol and are implicated in effects of ethanol, including tolerance, dependence, and withdrawal (Enoch. Pharmacol Biochem Behav. 2008, 90 (1), 95-104). GABA, GABA analogs that activate GABA_{A}R, or drugs that increase GABA tissue concentration by inhibiting GABA degradation pathways are used as antiseizure (antiepileptic) medications. In embodiments, oral compositions including genetically modified GAD-L. lactis of the disclosure can be used as an adjuvant in the treatment of epilepsy.

Epilepsy, or seizure disorder, involves disturbed nerve cell activity in the brain. A subject who has epilepsy can have the following symptoms: fainting or fatigue; rhythmic muscle contractions or muscle spasms; aura or pins and needles; seizures; amnesia; anxiety; depression; headache; sleepiness; staring spells; and temporary paralysis after a seizure. There is experimental and clinical evidence that GABA plays a role in epilepsy. Animal models of epilepsy demonstrate defective GABAergic function. Studies of human epileptic brain tissue show reductions in the following: GABA-mediated inhibition, activity of GAD, binding to GABA_{A}R and benzodiazepine sites, and GABA level in cerebrospinal fluid and brain tissue. GABA agonists suppress seizures, while GABA antagonists produce seizures. Drugs that inhibit GABA synthesis cause seizures. Benzodiazepines and barbiturates work by enhancing GABA-mediated inhibition. Additionally, drugs that increase synaptic GABA are potent anticonvulsants. Treatments for epilepsy include: medications; surgery; devices; and dietary changes. Medications include: nerve pain medication (e.g., topiramate, gabapentin, and pregabalin); sedatives (e.g., midazolam, phenobarbital, diazepam, and clonazepam); and anticonvulsants (e.g., carbamazepine, topiramate, phenytoin, felbamate, lamotrigine, valproic acid, primidone, levetiracetam, ethosuximide, gabapentin, and oxcarbazepine).

GABA antagonizes glutamatergic hyper-excitatory activity in the brain and excess glutamate is in part involved in brain cell death following ischemic stroke. In embodiments, oral compositions including genetically modified GAD-L. lactis of the disclosure can be used to treat post-stroke complications. In embodiments, oral compositions including genetically modified GAD-L. lactis of the disclosure can be used to treat stroke and complications.

A stroke occurs when blood supply to a part of the brain is interrupted or reduced, preventing brain tissue from receiving the needed oxygen and nutrients. Brain cells may die in minutes. Stroke may be caused by a blocked artery (ischemic stroke) or by leaking/bursting of a blood vessel (hemorrhagic stroke). Symptoms of a stroke include: trouble speaking and understanding what others are saying; paralysis or numbness of the face, arm, or leg; problems seeing in one or both eyes; headache; and trouble walking. Strokes may be prevented by generally following a healthy lifestyle including: controlling high blood pressure; lowering cholesterol and saturated fat in the diet; ceasing smoking; managing diabetes; maintaining a healthy weight; eating a diet rich in fruits and vegetables; exercising regularly; moderating alcohol consumption; treating obstructive sleep apnea; and avoiding illegal drugs. Preventive medications for stroke include anti-platelet drugs (e.g., aspirin, clopidogrel) and anti-coagulants (e.g., heparin, warfarin, dabagatrin, rivaroxaban, apixaban, edoxaban) to reduce blood clots and blood clotting. Complications from stroke include: brain edema, seizures, clinical depression, memory loss, changes in behavior and self-care ability, paralysis, difficulty talking or swallowing, pain, deep venous thrombosis, limb contractures, and urinary tract infection.

In embodiments, oral compositions including genetically modified GAD-L. lactis of the disclosure can be used to treat inflammatory autoimmune diseases including Inflammatory Bowel Diseases (IBD; Crohn's disease, ulcerative colitis); psoriatic arthritis; rheumatoid arthritis (RA); Type 1 diabetes; Type 2 Diabetes; Sjögren's syndrome; systemic lupus erythematosus (lupus or SLE); celiac disease; Graves' disease; Hashimoto's thyroiditis; Addison's disease; dermatomyositis; psoriasis; chronic inflammatory demyelinating polyneuropathy (CIDP); Guillain-Barre syndrome; myasthenia gravis; and vasculitis.

IBD encompasses disorders that involve chronic inflammation of the digestive tract. In particular embodiments, IBD includes ulcerative colitis (UC) and Crohn's disease. Symptoms common to both UC and Crohn's disease include: diarrhea, fever and fatigue, abdominal pain and cramping, bloody stools, reduced appetite, and weight loss. UC occurs in the large intestine (colon) and the rectum. Damage in UC is continuous (not patchy), and inflammation is present only in the innermost layer of the lining of the colon. Crohn's disease can affect any part of the GI tract, including from the mouth to the anus. In embodiments, Crohn's disease can affect the portion of the small intestine before the colon. Damaged areas in Crohn's disease are patchy and appear next to areas of healthy tissue, and inflammation can reach through multiple layers of the walls of the GI tract. IBD can be diagnosed using endoscopy and/or colonoscopy and imaging tools, including contrast radiography, magnetic resonance imaging (MRI), and/or computed tomography (CT). Stool samples can also be checked. IBD can currently be treated with medications including: aminosalicylates, corticosteroids (e.g., prednisone), immunomodulators, and biologics. Surgery can be used to remove damaged portions of the GI tract.

One animal model that can be used to study IBD is the 2,4,6-trinitrobenzenesulfonic acid (TNBS) model of IBD in mice. In this model, colitis is induced by the administration of TNBS via rectum. The colitis results in body weight loss and high mortality. The disease promotes intestinal epithelium disruption, inflammation, and shortness of colon length. Mice can be divided into groups to test the ability of genetically modified bacteria of the present disclosure to reduce or prevent body weight loss, reduce or prevent intestinal epithelium disruption, reduce or prevent inflammation, retain colon length, and/or improve survival. Control groups can include mice that drink normal water and/or mice that drink water containing genetically modified bacteria having a control plasmid (e.g., empty plasmid backbone) for a period of time (e.g., two weeks) before and after colitis induction. A control group of healthy mice can also be included in a study. A test group of mice can receive test genetically modified bacteria (e.g., bacteria genetically modified to produce GABA) in their drinking water for two weeks before and after colitis induction. The concentration of the genetically modified bacteria in the drinking water can be 5×10⁸ CFU/mouse. The drinking water can be deionized and autoclaved. Disease is induced in two stages: on day-7 (before TNBS application into the rectum), mice are pre-sensitized by shaving an area of skin on each mouse (e.g., a 1.5×1.5 cm square area) and applying TNBS to the shaved skin (e.g., 5% TNBS emulsion in acetone/olive oil). Sensitization causes mice to be more susceptible to disease. On day 0, the TNBS solution is administered into the rectum using a catheter/syringe set-up (e.g., 5% TNBS solution (weight/volume) in autoclaved water and 1 volume of absolute ethanol). Disease is monitored for five days, and survival rates and colon length can be determined.

Psoriatic arthritis is a form of inflammatory arthritis characterized by joint pain, swelling, and morning stiffness. It is associated with having psoriasis or a family history of psoriasis. Symptoms can include symptoms in joints, skin, and other symptoms. Joint symptoms include: pain or aching, tenderness, and/or swelling in one or more joints of, for example, hands, feet, wrists, ankles, knees; joint stiffness; reduced range of motion in affected joints; pain or stiffness in the lower back; tenderness, pain, or swelling where tendons and ligaments attach to the bone; and swelling of an entire finger or toe. Skin symptoms include: silver or gray scaly spots on the scalp, elbows, knees, and/or the lower spine; small, round spots (papules) that are raised and sometimes scaly on the arms, legs and torso; pitting of the nails; and detachment or lifting of fingernails or toenails. Other symptoms include: inflammation of the eye (iritis or uveitis); fatigue; and anemia. Psoriatic arthritis may be diagnosed based on a patient's medical history, physical exam, blood tests, and/or X-rays of the affected joints. In embodiments, blood tests include tests to detect rheumatoid factor antibody (anti-CCP), HLA-B27, sedimentation rate (ESR), and C-reactive protein (CRP). Treatments for psoriatic arthritis include nonsteroidal anti-inflammatory drugs (NSAIDs) such as aspirin, ibuprofen, or naproxen; corticosteroids (oral and injectable forms); disease modifying anti-rheumatic drugs (DMARDs); biologics; exercise; heat and cold therapy; joint protection and energy conservation; splinting of joints; surgery on damaged joints; or a combination thereof.

Rheumatoid arthritis (RA) involves chronic inflammation in joints on both sides of the body (e.g., both hands, wrists, and/or knees), which helps distinguish it from other types of arthritis. In embodiments, RA may occasionally affect other parts of the body, including the skin, eyes, lungs, heart, blood, nerves, or kidneys. In embodiments, cartilage may be destroyed and/or joints deformed. Symptoms can include joint pain and swelling; stiffness; and/or fatigue. RA may be diagnosed based on morning stiffness that lasts at least one hour and has been present for at least six weeks; swelling of three or more joints for at least six weeks; swelling of the wrist, hand, or finger joints for at least six weeks; swelling of the same joints on both sides of the body; changes in hand x-rays that are hallmarks of rheumatoid arthritis; rheumatoid nodules (lumps) of the skin; blood test that is positive for rheumatoid factor and/or anti-citrullinated peptide/protein antibodies;

or a combination thereof. Treatments for RA include medications, rest, exercise, physical therapy/occupational therapy, and surgery to correct damage to the joint. In embodiments, medications for RA include: NSAIDs, such as aspirin, ibuprofen, or naproxen; corticosteroids (oral and injectable forms); COX-2 inhibitor (celecoxib [Celebrex®]); DMARDs such as hydroxychloroquine (Plaquenil), methotrexate (Rheumatrex®, Trexall®), sulfasalazine (Azulfidine®), and leflunomide (Arava®); and biologic agents, such as infliximab (Remicade®), etanercept (Enbrel®), adalimumab (Humira®), certolizumab (Cimzia®), golimumab (Simponi®), tocilizumab (Actemra®), rituximab (Rituxan®), abatacept (Orencia®), anakinra (Kineret®), and tofacitinib (Xeljanz®).

Type 1 diabetes, also known as juvenile diabetes, is a genetic disorder where the immune system attacks and destroys beta cells in the pancreas that produce insulin. Insulin is a hormone that helps entry of glucose into cells for energy. Symptoms can include frequent urination, abnormal thirst, unexplained weight loss, frequent exhaustion, bedwetting, vaginal yeast infection, slowly healing sores, dry itchy skin, tingling sensation in the feet, and blurry eyesight. Type 1 diabetes may be diagnosed based on blood tests to assess glucose levels and/or autoantibodies, and/or a urine test to test for ketones. Treatments for Type 1 diabetes is lifelong and include: daily insulin injections; a healthy diet; regular exercise; and management of stress.

Type 2 diabetes is characterized by an inability of the body to produce enough insulin, or there is an increase in insulin resistance. The disorder is usually diet-related and develops over time. Symptoms can include frequent urination, hunger, fatigue, and blurred vision. Type 2 diabetes may be diagnosed based on a glycated hemoglobin (A1C) test, which indicates the average blood sugar level for the past 2-3 months. Treatments for Type 2 diabetes include insulin therapy, diet, exercise, and medication. Medications can include: an anti-diabetic medication to control blood sugar (e.g., saxagliptin, glyburide, metformin, glipizide, rosiglitazone, pioglitazone, and glimepiride); a statin to decrease the liver's production of harmful cholesterol; and insulin.

Sjögren's syndrome is an autoimmune disorder that reduces the amount of moisture produced by glands in the eyes and mouth. Symptoms can include extremely dry eyes and mouth; joint pain; muscle pain; abnormal sense of taste; burning or redness, and/or grittiness in eyes; blurry vision; difficulty chewing, swallowing or talking; dry cough or hoarseness; dry, itchy skin; enlarged salivary glands; fatigue; tooth decay or early tooth loss; and/or vaginal dryness. Sjögren's syndrome may be diagnosed based on: blood tests to detect anti-nuclear antibodies (ANA), anti-Sjögren's syndrome antibodies (anti-SSA, also called anti-Ro), anti-Sjögren's syndrome type B (anti-SSB, also called anti-La), and/or rheumatoid factor antibody; biopsy of a salivary gland or the inside of the lip to detect inflammation; eye exam to measure tear production; imaging tests including sialometry to measure how much saliva is produced by using X-rays that can see dye injected into salivary glands, and salivary scintigraphy, a way to track how long it takes for a radioactive isotope to travel from an injection point in the vein to the salivary glands; health history; or a combination thereof. Treatments for Sjögren's syndrome include ones to treat particular symptoms. Treatments for dry eyes include: artificial tears; prescription eye drops (e.g., cyclosporine (Restasis®) and lifitegrast (Xiidra®)); punctal plugs that block tear ducts so tears stay on the eyes; surgery to close tear ducts permanently; autologous serum drops, which involve mixing a subject's blood serum with a sterile liquid solution to create customized artificial tears; or a combination thereof. Treatment for dry mouth include: saliva producers such as gum and hard candies that contain sweeteners like sorbitol or xylitol, prescription sorbitol oral lozenges, and prescription sorbitol oromucosal solutions; prescription medications such as pilocarpine (Salagen®) and cevimeline (Evoxac®) pills to increase the natural production of saliva; dental care; or a combination thereof. Treatment for joint and organ problems include: over-the-counter pain relievers such as acetaminophen and NSAIDs (e.g., ibuprofen, naproxen); anti-rheumatics (e.g., hydroxychloroquine); immunosuppressants to lessen inflammation and prevent organ damage; steroids (e.g., prednisone); antifungals to treat yeast overgrowth. Treatment for vaginal dryness include: vaginal moisturizers or lubricants; using unscented soaps for cleansing; and/or vaginal estrogen therapy.

Systemic lupus erythematosus (lupus or SLE) is a chronic autoimmune disease that can cause inflammation and pain throughout the body. SLE may involve joint pain, skin sensitivities and rashes, and issues with internal organs (brain, lungs, kidneys and heart). In embodiments, subjects with SLE may have: dangerous reductions in red blood cells, white blood cells, and/or platelets; blood clots; arthritis; kidney disease; problems associated with the brain such as depression, confusion, seizures, or strokes; and/or inflammation of the pericardium or pleura. Symptoms can include joint pain; muscle pain; rashes; fever; sensitivity to sunlight; hair loss; mouth sores; dry eyes; fatigue; chest pain; stomach pain; shortness of breath; swollen glands; headaches; confusion; depression; issues with the kidneys, heart or lungs; seizures; blood clots; anemia; and/or Raynaud's phenomenon, where the small blood vessels in the fingers and toes constrict in response to temperature extremes, certain occupational exposures, or excitement. SLE may be diagnosed based on family history and blood tests to detect ANA, low blood cell counts, anemia, or other abnormalities. Medications to treat SLE include steroids (corticosteroids, including prednisone); hydroxychloroquine to control skin and joint disease, fatigue, and/or mouth sores; azathioprine (Imuran®); methotrexate (Rheumatrex®) to suppress the immune system; chemotherapy drugs such as cyclophosphamide (Cytoxan®) and mycophenolate mofetil (CellCept®); monoclonal antibodies that reduce the activity of white blood cells (lymphocytes) that make autoantibodies (belimumab (Benlysta®); rituximab (Rituxan®)); or a combination thereof.

Celiac disease is a digestive and autoimmune disorder that can damage the small intestine. In embodiments, Celiac disease may be triggered by the protein gluten found in grains like wheat, barley, and rye. Symptoms can include bloating, gas, diarrhea, anemia, depression, and growth issues in children. Celiac disease may be diagnosed based on medical history, a blood test to detect antibodies to gluten, tests to detect nutritional deficiencies (e.g., iron), and a biopsy of the small intestine. Treatment for Celiac disease include avoiding food containing gluten.

Graves' disease is an autoimmune disease characterized by an overactive thyroid gland. The immune system is triggered to overproduce an antibody called thyroid-stimulating immunoglobulin (TSI). The thyroid gland produces hormones that regulate metabolism. Too much thyroid hormone can damage the heart and other organs. Symptoms can include difficulty sleeping, enlarged thyroid, eye inflammation, heart arrhythmia, fatigue, hand tremors, heat intolerance, irritability, muscle weakness, and unexplained weight loss. Graves' disease may be diagnosed based on thyroid blood tests to measure TSI, radioactive iodine uptake test, and a thyroid scan with radioactive technetium to image the thyroid. Treatments for Graves' disease include: beta blockers to protect the heart; antithyroid medications (e.g., methimazole, propylthiouracil); radiation therapy to destroy thyroid gland cells; and surgery to remove all or part of the thyroid gland.

Hashimoto's thyroiditis is characterized by hypothyroidism, or insufficient production of thyroid hormones by the thyroid gland. The immune system makes antibodies that attack and damage thyroid tissue. Symptoms can include fatigue, weight gain, feeling cold, joint stiffness, muscle pain, constipation, depression, puffy eyes or face, dry skin, thinning hair, irregular periods, memory problems, and slow heartbeat. Hashimoto's thyroiditis may be diagnosed based on: a thyroid stimulating hormone test, a free T4 test, an antithyroid antibody test, and/or an ultrasound of the thyroid. Treatments for Hashimoto's thyroiditis include administering synthetic thyroid hormone (levothyroxine).

Addison's disease, also known as primary adrenal insufficiency, is characterized by insufficient production of the hormones cortisol and aldosterone by the adrenal glands that are located at the top of the kidneys. Cortisol helps the body respond to stress, and maintains blood pressure, heart function, the immune system, and blood glucose levels. Aldosterone regulates the balance of sodium and potassium in the blood, which influences the amount of fluid removed by the kidneys, and ultimately affect blood volume and blood pressure. In Addison's disease, the immune system attacks the cortex of the adrenal glands, where cortisol and aldosterone are produced. Symptoms can include: abdominal pain, abnormal menstrual periods, craving for salty food, dehydration, depression, diarrhea, irritability, dizziness, loss of appetite, low blood glucose, low blood pressure, muscle weakness, nausea, patches of dark skin, sensitivity to cold, unexplained weight loss, vomiting, and worsening fatigue. Addison's disease may be diagnosed based on medical history; physical exam; blood tests to measure levels of sodium, potassium, cortisol, and adrenocorticotropic hormone (ACTH); ACTH stimulation test; X-rays to detect calcium deposits on the adrenal glands; and computed tomography (CT) scan to evaluate the adrenal glands. Treatments for Addison's disease include prescription hormones to replace cortisol (e.g., hydrocortisone) and aldosterone (e.g., fludrocortisone acetate).

Dermatomyositis is an inflammatory muscle disease that affects the skin. Symptoms can include: reddish or bluish-purple patches; purple spots on bony areas like knuckles; discoloration with swelling around the eyes; ragged cuticles; and a red rash on the face, neck, shoulders, upper chest, and/or elbows. Dermatomyositis may be diagnosed based on: blood tests to detect increased amounts of muscle enzymes such as creatine kinase and lactate dehydrogenase; blood tests to detect autoantibodies; skin biopsy of the rash; biopsy of an affected muscle; electromyography testing; and magnetic resonance imaging (MRI) scan of muscles. Treatments for dermatomyositis include: steroids (e.g., prednisone); immunosuppressants (e.g., methotrexate, azathioprine, cyclophosphamide, chlorambucil, cyclosporine, tacrolimus, mycophenolate, and rituximab); intravenous immunoglobulins (IVIG) to slow down the autoimmune process; and physical therapy to preserve muscle function.

Psoriasis is a skin disorder characterized by itchiness and thick, scaly patches of skin (plaques). The patches can occur anywhere on the body but tend to affect: elbows and knees; face, scalp, and inside the mouth, fingernails and toenails; genitals; lower back; and palms and feet. The immune system overreacts, causing inflammation and abnormal growth of new skin cells. Symptoms can include itchiness, cracked dry skin, scaly scalp, skin pain, pitted nails, and joint pain. Psoriasis may be diagnosed based on microscopy of a skin sample. Treatments for psoriasis include steroid creams, moisturizers, anthralin to slow skin cell production, medicated lotions and/or UV light therapy for scalp psoriasis, vitamin D3 ointment, and vitamin A or retinoid creams.

Chronic inflammatory demyelinating polyneuropathy (CIDP) is a neurological disorder that is characterized by inflammation of nerves and nerve roots. In some instances, myelin, the protective covering of nerves, may be damaged. CIDP is distinct from Guillain-Barre syndrome because it is not brought on by an infection. Symptoms can include numbness and pain; slow reflexes; fatigue; and weakness in arms and legs. There is no test for CIDP, so health care providers rely on physical examination and tests to rule out other diseases. Treatments for CIDP include corticosteroids, IVIG, plasma exchange, immunotherapy to reduce immune system attacks on myelin, and stem cell transplant to “reset” the immune system.

Guillain-Barre syndrome (GBS) involves the immune system attacking the body's nerves so that a person suffering from GBS has challenges controlling their muscles or sensing the environment. GBS is usually triggered by a viral or bacterial infection. Symptoms can include numbness or tingling in the hands and feet; back pain; muscle weakness; difficulty breathing; difficulty swallowing; and heart rate or blood pressure problems. GBS may be diagnosed based on a lumbar puncture to sample the cerebrospinal fluid and electromyography to test the function of nerves and muscles. Treatments for GBS include IVIG to slow down the autoimmune process and plasma exchange to eliminate/reduce the autoantibodies attacking the nerves.

Myasthenia gravis affects the neuromuscular system. The autoimmune form is the most common form of the disease. The immune system develops antibodies that destroy acetylcholine receptors present in muscles, leading to blocked nerve-muscle communication. Symptoms can include double vision, drooping eyelids, difficulty speaking, chewing, or swallowing, limb weakness, and trouble walking. Myasthenia gravis may be diagnosed based on: ice pack test on drooping eyelids; antibody tests to detect high levels of acetylcholine receptor antibodies or muscle-specific kinase antibodies; MRI and/or CT scans to check for thymus gland problems; and electromyograms. Treatments for myasthenia gravis include: medications (e.g., cholinesterase inhibitors to boost nerve-muscle signaling; immunosuppressants to decrease inflammation); immunosuppressing monoclonal antibodies; IVIG; plasma exchange; and surgery to remove the thymus gland.

Vasculitis is characterized by inflammation of the body's blood vessels, including capillaries, medium-size blood vessels, and large blood vessels like the aorta. Inflamed blood vessels become weakened and stretch in size, which can lead to aneurysms or rupture. Symptoms can include skin rashes, fatigue, weakness, fever, joint pain, abdominal pain, kidney problems, nerve problems, and cough/shortness of breath. Vasculitis may be diagnosed based on: blood tests to detect low red blood cell count, high white blood cell count, high platelet count, and signs of kidney or liver problems; blood tests to detect antibodies associated with vasculitis; X-rays; tissue biopsies; and scans of blood vessels and heart. Treatments for vasculitis include corticosteroids and immunosuppressants.

In embodiments, oral compositions including genetically modified GAD-L. lactis of the disclosure can be used to treat behavioral and mood disorders, such as anxiety, post-traumatic stress disorder, and autism spectrum disorders (ASD); and cachexia secondary to chronic inflammation (e.g., as seen in cancer). For example, the number of GABA_{A} receptors is reduced in the temporal lobe of patients with generalized anxiety disorder.

Autism, or autism spectrum disorder (ASD), refers to a developmental disability that involves significant social, communication, and behavioral challenges. A subject having ASD may have the following symptoms: does not point at objects to show interest, does not look at objects when another person points at the objects, has trouble relating to others, avoids eye contact, has trouble understanding others' feelings, appear unaware of others, prefers to not cuddle, repeat or echo words or phrases, does not “pretend” play, has trouble expressing needs, repeats actions, has trouble adapting to changes, has unusual reactions to sensory properties of objects, and/or lose skills they once had. Defects in glutamate and GABA signaling may underlie ASD symptoms. Diagnosis of ASD can be challenging, as there is no medical test. Health care providers look at a subject's behavior and development to make a diagnosis. There is currently no cure for ASD. Treatment services are directed to improving development and/or social skills.

Post-traumatic stress disorder (PTSD) develops in an individual who has experienced a shocking, scary, or dangerous event. People suffering from PTSD may continue to feel stressed or frightened after the trauma even when they are not in danger. Symptoms may include re-experiencing symptoms (e.g., flashbacks, racing heart, sweating, bad dreams, frightening thoughts), avoidance symptoms (e.g., staying away from places, events, or objects that remind them of the traumatic event; avoiding thoughts related to the traumatic event), reactivity symptoms (e.g., easily startled, feeling tense, difficulty sleeping, having angry outbursts), and cognition and mood symptoms (e.g., trouble remembering features of the traumatic event; negative thoughts; feeling guilt or blame; loss of interest in enjoyable activities). A diagnosis of PTSD may be given if symptoms last more than one month and are severe enough to interfere with relationships or work. Treatments for PTSD include: medication (selective serotonin re-uptake inhibitors such as fluoxetine, sertraline, and paroxetine) and/or psychotherapy. Patients with PTSD have lower levels of GABA in the medial prefrontal cortex.

Cachexia refers to the progressive loss of skeletal muscle mass and adipose tissue. Criteria to diagnose cachexia include weight loss in the presence of underlying illness of >5% in ≤12 months, or weight loss>2% in individuals with a low body-mass index (<20 kg/m²) or low muscle mass, and the presence of decreased muscle strength, fatigue or anorexia, and elevated inflammatory serum markers. This syndrome is seen in patients with cancer and with chronic illnesses including chronic heart failure, chronic kidney disease, chronic obstructive pulmonary disease, and rheumatoid arthritis. Cachexia is associated with decreased survival and quality of life in these diseases. Cachexia is characterized by systemic inflammation and immune cell infiltration in tissues. Existing treatments include appetite stimulants and focus on alleviation of symptoms rather than prolongation of life. Recent therapies for cachexia involve combination therapy that involves diet modification and/or exercise and pharmaceutical agents, such as megestrol acetate, medroxyprogesterone, ghrelin, and omega-3-fatty acid.

An “effective amount” is the amount of a composition necessary to result in a desired physiological change in a subject. Effective amounts are often administered for research purposes. Representative effective amounts disclosed herein can reduce clinical symptoms or reduce weight loss in an EAE mouse model.

A “prophylactic treatment” includes a treatment administered to a subject who does not display signs or symptoms of a disease or nutritional deficiency or displays only early signs or symptoms of a disease or nutritional deficiency, such that treatment is administered for the purpose of diminishing, preventing, or decreasing the risk of developing the disease or nutritional deficiency further. Thus, a prophylactic treatment functions as a preventative treatment against the development of diseases or nutritional deficiencies.

As an example of a prophylactic treatment, a composition disclosed herein can be administered to a subject who is at risk of developing an anxiety disorder. An effective prophylactic treatment of an anxiety disorder occurs when symptoms such as restlessness; fatigue; difficulty concentrating; irritability or explosive anger; muscle tension; sleep disturbances; and/or personality changes are prevented or reduced in frequency or duration as measured by a standard subjective or objective anxiety disorder assessment. Anxiety disorder can be assessed by a number of tools including: the Subjective Units of Distress Scale (SUDs), a self-assessment tool that measures the intensity of distress or nervousness in people with social anxiety on a scale from 0 to 100 (Benjamin et al. Behav Cogn Psychother. 2010; 38 (4): 497-504); the Beck Anxiety Inventory (BAI; Beck et al. J Consult Clin Psychol. 1988; 56 (6): 893-897), focusing on somatic and panic-like symptoms of anxiety; the State-Trait Anxiety Inventory (STAI-T; Julian L J. Arthritis Care Res (Hoboken) 2011; 63 (011): 10.1002/acr.20561), focusing on two subscales, current state of anxiety and anxiety proneness; the Hospital Anxiety and Depression Scale-Anxiety (HADS-A; Zigmond and Snaith. Acta Psychiatr Scand. 1983; 67 (6): 361-370), involving a 4-point Likert scale ranging from 0 to 3 to evaluate common dimensions of anxiety; and the Generalized Anxiety Disorder 7-item scale (GAD-7; Spitzer et al. Arch Intern Med. 2006; 166 (10): 1092-1097), which includes a short questionnaire that is useful for screening, with a score of 0-4 indicating minimal anxiety, a score of 5-9 indicating mild anxiety, a score of 10-14 indicating moderate anxiety, and a score greater than 15 indicating severe anxiety.

As another example of a prophylactic treatment, a composition disclosed herein can be administered to a subject who is at risk of having epilepsy including epileptic seizures. An effective prophylactic treatment of epileptic seizures occurs when the number or severity of seizures per month is reduced by at least 10% or in embodiments, by 25%, as measured by tests, such as ictal scalp electroencephalogram (EEG) or ambulatory EEG monitoring and home video recording. A neurological exam, a blood test, a lumbar puncture, and/or neuroimaging tests (e.g., MRI, CT, positron emission tomography (PET), and/or single-photon emission computerized tomography (SPECT)) can be used to confirm or rule out causes of epileptic seizure such as hemorrhage, infection, tumor and disorders related to cerebrospinal fluid hypertension or hypotension.

A “therapeutic treatment” includes a treatment administered to a subject who has a disease or nutritional deficiency and is administered to the subject for the purpose of curing or reducing the severity of the disease or nutritional deficiency.

As one example of a therapeutic treatment, a composition disclosed herein can be administered to a subject who has multiple sclerosis (MS). An effective therapeutic treatment of MS occurs when the score in a standard walk test improves by 10% and in particular embodiments, by 25%. Rating scales, performance tests, and patient self-report questionnaires can be used to evaluate walking in MS. Rating scales include: the Kurtzke expanded disability status scale (Kurtzke EDSS; Kurtzke J F. Neurology. 1983; 33 (11): 1444-1452), which involves a numerical score from 0 to 10, with walking assessed in the middle range of the scale from 4.7 to 7.5, and consideration of maximum distance walked and use of an assistive device; the Hauser Ambulation Index (Hauser et al. N Engl J Med. 1983; 308 (4): 173-180), which involves a scale from 0 to 9, with scoring dependent on the need for an assistive mobility device and on the ability and time required to walk 25 feet; the Dynamic Gait Index (DGI; Shumway-Cook and Wollacott. Motor Control: Theory and Practical Applications. Baltimore, MD: Williams and Wilkins; 1995) incorporates walking, stair climbing, and balance; and the Rivermead Visual Gait Assessment (RVGA; Lord et al. Clin Rehabil. 1998; 12 (2): 107-119), a qualitative gait analysis tool that considers deviations during the stance and swing phases of gait, including a total score that can range from 0 to 59, with higher scores indicating more severe deviations from normal. Timed walking tests are objective assessments of MS and can include: a timed 25-foot walk (or a timed walk of another specified distance; Fischer et al. Mult Scler. 1999; 5 (4): 244-250); the 6-minute walk test, which records the maximum distance walked in 6 minutes (Goldman et al. Mult Scler. 2008; 14 (3): 383-390); the timed Up and Go test (TUG; Podsiadlo and Richardson. J Am Geriatr Soc. 1991; 39 (2): 142-148) considers the time required for a person to rise from a chair, walk 3 m, turn around, walk back to the chair, and sit down; and the Six Spot Step Test (SSST; Nieuwenhuis et al. Mult Scler. 2006; 12 (4): 495-500), which considers lower extremity function.

Another example of a therapeutic treatment includes administration of a composition disclosed herein to a subject who has anxiety. An effective therapeutic treatment of anxiety occurs when the severity of the anxiety is reduced or relieved completely and/or more quickly measured by a standard subjective or objective anxiety assessment. Assessments of anxiety can be conducted as described herein.

Another example of a therapeutic treatment includes administration of a composition disclosed herein to a subject experiencing epilepsy including epileptic seizures. An effective therapeutic treatment of epilepsy occurs when the severity or number of epileptic seizures is reduced or relieved completely and/or more quickly as measured by a standard subjective or objective epileptic seizure assessment. Assessments of epileptic seizures can be conducted as described herein.

Another example of a therapeutic treatment includes administration of a composition disclosed herein to a subject who has autism spectrum disorder (ASD). An effective therapeutic treatment of ASD occurs when social communication and interaction are improved and/or repetitive patterns of behavior are reduced or eliminated. As examples, symptoms such as: limited or no verbal or facial communication; no interest in others; repetitive behaviors; echolalia; and extreme sensitivity to noise are improved as measured by a standard subjective or objective ASD assessment. Assessments of ASD include: ages and stages questionnaire that parents can complete, which include questions on gross motor, fine motor, problem-solving, and personal adaptive skills and provides a pass/fail score; Communication and Symbolic Behavior Scales (CSBS), which assesses children up to the 24-month level; the Parents' Evaluation of Developmental Status (PEDS), which is a parent interview form that can be used as a screening and surveillance tool; the Modified Checklist for Autism in Toddlers (MCHAT), a parent-completed questionnaire that identifies children at risk for autism; and the Screening Tool for Autism in Toddlers and Young Children (STAT), an interactive tool including 12 activities that assess play, communication, and imitation skills.

Therapeutic treatments can be distinguished from effective amounts based on the presence or absence of a research component to the administration. As will be understood by one of ordinary skill in the art, however, in human clinical trials effective amounts, prophylactic treatments and therapeutic treatments can overlap.

For administration, therapeutically effective amounts (also referred to herein as doses) can be initially estimated based on results from in vitro assays and/or animal model studies. Such information can be used to more accurately determine useful doses in subjects of interest.

The actual dose amount administered to a particular subject can be determined by the subject, a physician, veterinarian, or researcher taking into account parameters such as physical, physiological and psychological factors including target, body weight, condition, previous or concurrent therapeutic interventions, and/or idiopathy of the subject.

In embodiments, therapeutically effective amounts include 1×10⁷ colony forming units (CFU), 1.5×10⁷ CFU, 2×10⁷ CFU, 2.5×10⁷ CFU, 3×10⁷ CFU, 3.5×10⁷ CFU, 4×10⁷ CFU, 4.5×10⁷ CFU, 5×10⁷ CFU, 5.5×10⁷ CFU, 6×10⁷ CFU, 6.5×10⁷ CFU, 7×10⁷ CFU, 7.5×10⁷ CFU, 8×10⁷ CFU, 8.5×10⁷ CFU, 9×10⁷ CFU, 9.5×10⁷ CFU, 1×10⁸ CFU, 1.5×10⁸ CFU, 2×10⁸ CFU, 2.5×10⁸ CFU, 3×10⁸ CFU, 3.5×10⁸ CFU, 4×10⁸ CFU, 4.5×10⁸ CFU, 5×10⁸ CFU, 5.5×10⁸ CFU, 6×10⁸ CFU, 6.5×10⁸ CFU, 7×10⁸ CFU, 7.5×10⁸ CFU, 8×10⁸ CFU, 8.5×10⁸ CFU, 9×10⁸ CFU, 9.5×10⁸ CFU, 1×10⁹ CFU, 1.5×10⁹ CFU, 2×10⁹ CFU, 2.5×10⁹ CFU, 3×10⁹ CFU, 3.5×10⁹ CFU, 4×10⁹ CFU, 4.5×10⁹ CFU, 5×10⁹ CFU, 5.5×10⁹ CFU, 6×10⁹ CFU, 6.5×10⁹ CFU, 7×10⁹ CFU, 7.5×10⁹ CFU, 8×10⁹ CFU, 8.5×10⁹ CFU, 9×10⁹ CFU, 9.5×10⁹ CFU, 1×10¹⁰ CFU, or more.

In embodiments, therapeutically effective amounts include 1×10⁷ CFU/mL, 1.5×10⁷ CFU/mL, 2×10⁷ CFU/mL, 2.5×10⁷ CFU/mL, 3×10⁷ CFU/mL, 3.5×10⁷ CFU/mL, 4×10⁷ CFU/mL, 4.5×10⁷ CFU/mL, 5×10⁷ CFU/mL, 5.5×10⁷ CFU/mL, 6×10⁷ CFU/mL, 6.5×10⁷ CFU/mL, 7×10⁷ CFU/mL, 7.5×10⁷ CFU/mL, 8×10⁷ CFU/mL, 8.5×10⁷ CFU/mL, 9×10⁷ CFU/mL, 9.5×10⁷ CFU/mL, 1×10⁸ CFU/mL, 1.5×10⁸ CFU/mL, 2×10⁸ CFU/mL, 2.5×10⁸ CFU/mL, 3×10⁸ CFU/mL, 3.5×10⁸ CFU/mL, 4×10⁸ CFU/mL, 4.5×10⁸ CFU/mL, 5×10⁸ CFU/mL, 5.5×10⁸ CFU/mL, 6×10⁸ CFU/mL, 6.5×10⁸ CFU/mL, 7×10⁸ CFU/mL, 7.5×10⁸ CFU/mL, 8×10⁸ CFU/mL, 8.5×10⁸ CFU/mL, 9×10⁸ CFU/mL, 9.5×10⁸ CFU/mL, 1×10⁹ CFU/mL, 1.5×10⁹ CFU/mL, 2×10⁹ CFU/mL, 2.5×10⁹ CFU/mL, 3×10⁹ CFU/mL, 3.5×10⁹ CFU/mL, 4×10⁹ CFU/mL, 4.5×10⁹ CFU/mL, 5×10⁹ CFU/mL, 5.5×10⁹ CFU/mL, 6×10⁹ CFU/mL, 6.5×10⁹ CFU/mL, 7×10⁹ CFU/mL, 7.5×10⁹ CFU/mL, 8×10⁹ CFU/mL, 8.5×10⁹ CFU/mL, 9×10⁹ CFU/mL, 9.5×10⁹ CFU/mL, 1×10¹⁰ CFU/mL, or more.

Therapeutically effective amounts can be achieved by administering single or multiple doses during the course of a treatment regimen (e.g., hourly, every 2 hours, every 3 hours, every 4 hours, every 6 hours, every 9 hours, every 12 hours, every 18 hours, daily, every other day, every 3 days, every 4 days, every 5 days, every 6 days, weekly, every 2 weeks, every 3 weeks, or monthly).

One or more active agent(s) can be administered simultaneously or within a selected time window, such as within 10 minutes, 1 hour, 3 hour, 10 hour, 15 hour, 24 hour, or 48 hour time windows or when the complementary active agent(s) is within a clinically-relevant therapeutic window.

A composition including genetically modified probiotic bacteria producing GABA can be administered orally. In embodiments, oral administration includes providing an oral dosage form that can be swallowed and the active ingredient absorbed through the gastrointestinal tract.

Variants. Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

Functional variants include one or more residue additions or substitutions that do not substantially impact the physiological effects of the protein. Functional fragments include one or more deletions or truncations that do not substantially impact the physiological effects of the protein. A lack of substantial impact can be confirmed by observing experimentally comparable results in an activation study or a binding study. Functional variants and functional fragments of intracellular domains (e.g., intracellular signaling domains) transmit activation or inhibition signals comparable to a wild-type reference when in the activated state of the current disclosure. Functional variants and functional fragments of binding domains bind their cognate antigen or ligand at a level comparable to a wild-type reference.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157 (1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within +2 is preferred, those within +1 are particularly preferred, and those within +0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within +2 is preferred, those within +1 are particularly preferred, and those within +0.5 are even more particularly preferred.

As outlined above, amino acid substitutions may be based on the relative similarity of the amino acid side-chain substituents, for example, their hydrophobicity, hydrophilicity, charge, size, and the like.

As indicated elsewhere, variants of gene sequences can include codon optimized variants, sequence polymorphisms, splice variants, and/or mutations that do not affect the function of an encoded product to a statistically significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including (but not limited to) those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Closing Paragraphs. Each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability of genetically modified probiotic bacteria described herein to produce GABA.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006).

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### EXEMPLARY EMBODIMENTS

1. A genetically modified probiotic bacterium including a heterologous gene encoding a glutamic acid decarboxylase and a heterologous gene encoding a glutamate/GABA antiporter.

2. The genetically modified probiotic bacterium of embodiment 1, wherein the probiotic bacterium includes a lactic acid bacterium.

3. The genetically modified probiotic bacterium of embodiment 2, wherein the lactic acid bacterium belongs to a genus selected from the group consisting of Bifidobacterium, Carnobacterium, Enterococcus, Lactobacillus, Lactococcus, Leuconostoc, Oenococcus, Pediococcus, Propionibacterium, Streptococcus, Tetragenococcus, Vagococcus, and Weissella.

4. The genetically modified probiotic bacterium of embodiment 2 or 3, wherein the lactic acid bacterium belongs to the genus Bifidobacterium and include Bifidobacterium breve, Bifidobacterium longum, Bifidobacterium pseudolongum, Bifidobacterium animalis, Bifidobacterium adolescentis, Bifidobacterium angulatum, Bifidobacterium dentium, Bifidobacterium bifidum, Bifidobacterium lactis, Bifidobacterium catenulatum, Bifidobacterium pseudocatenulatum, Bifidobacterium magnum, or a combination thereof.

5. The genetically modified probiotic bacterium of any of embodiments 2-4, wherein the lactic acid bacterium belongs to the genus Carnobacterium and include Carnobacterium alterfunditum, Carnobacterium divergens, Carnobacterium funditium, Carnobacterium gallinarum, Carnobacterium iners, Carnobacterium inhibens, Carnobacterium jeotgali, Carnobacterium maltaromaticum, Carnobacterium mobile, Carnobacterium piscicola, Carnobacterium pleistocenium, Carnobacterium viridans, or a combination thereof.

6. The genetically modified probiotic bacterium of any of embodiments 2-5, wherein the lactic acid bacterium belongs to the genus Enterococcus and include Enterococcus alcedinis, Enterococcus aquimarinus, Enterococcus asini, Enterococcus avium, Enterococcus bulliens, Enterococcus burkinafasonensis, Enterococcus caccae, Enterococcus camelliae, Enterococcus canintestini, Enterococcus canis, Enterococcus casseliflavus, Enterococcus cecorum, Enterococcus columbae, Enterococcus crotali, Enterococcus devriesei, Enterococcus diestrammenae, Enterococcus dispar, Enterococcus durans, Enterococcus eurekensis, Enterococcus faecalis, Enterococcus faecium, Enterococcus gallinarum, Enterococcus gilvus, Enterococcus haemoperoxidus, Enterococcus hermanniensis, Enterococcus hirae, Enterococcus hulanensis, Enterococcus italicus, Enterococcus lactis, Enterococcus lemanii, Enterococcus malodoratus, Enterococcus massiliensis, Enterococcus mediterraneensis, Enterococcus moraviensis, Enterococcus mundtii, Enterococcus olivae, Enterococcus pallens, Enterococcus phoeniculicola, Enterococcus plantarum, Enterococcus pseudoavium, Enterococcus quebecensis, Enterococcus raffinosus, Enterococcus ratti, Enterococcus rivorum, Enterococcus rotai, Enterococcus saccharolyticus, Enterococcus saigonensis, Enterococcus silesiacus, Enterococcus sulfureus, Enterococcus solitarius, Enterococcus songbeiensis, Enterococcus termitis, Enterococcus thailandicus, Enterococcus ureasiticus, Enterococcus ureilyticus, Enterococcus viikkiensis, Enterococcus villorum, Enterococcus wangshanyuanii, Enterococcus xiangfangensis, Enterococcus xinjiangensis, or a combination thereof.

7. The genetically modified probiotic bacterium of any of embodiments 2-6, wherein the lactic acid bacterium belongs to the genus Lactobacillus and include Lactobacillus acidophilus, Lactobacillus crispatus, Lactobacillus amylovorus, Lactobacillus brevis, Lactobacillus buchneri, Lactobacillus casei, Lactobacillus curvatus, Lactobacillus delbrueckii subsp. bulgaricus, Lactobacillus farciminis, Lactobacillus fermentum, Lactobacillus futsaii, Lactobacillus gallinarum, Lactobacillus gasseri, Lactobacillus graminis, Lactobacillus helveticus, Lactobacillus johnsonii, Lactobacillus mali, Lactobacillus mucosae, Lactobacillus namurensis, Lactobacillus otakiensis, Lactobacillus paracasei, Lactobacillus paralimentarius, Lactobacillus plantarum, Lactobacillus reuteri, Lactobacillus rhamnosus, Lactobacillus rossiae, Lactobacillus sakei, Lactobacillus salivarius, Lactobacillus viridescens, Lactobacillus zeae, or a combination thereof.

8. The genetically modified probiotic bacterium of any of embodiments 2-7, wherein the lactic acid bacterium belongs to the genus Lactococcus and include Lactococcus chungangensis, Lactococcus cremoris, Lactococcus formosensis, Lactococcus fujiensis, Lactococcus garvieae, Lactococcus garvieae subsp. garvieae, Lactococcus garvieae subsp. Bovis, Lactococcus hircilactis, Lactococcus lactis, Lactococcus lactis subsp. cremoris, Lactococcus lactis subsp. hordniae, Lactococcus lactis subsp. lactis, Lactococcus lactis subsp. tructae, Lactococcus laudensis, Lactococcus nasutitermitis, Lactococcus petauri, Lactococcus piscium, Lactococcus plantarum, Lactococcus raffinolactis, Lactococcus taiwanensis, or a combination thereof.

9. The genetically modified probiotic bacterium of any of embodiments 2-8, wherein the lactic acid bacterium belongs to the genus Leuconostoc and include Leuconostoc carnosum, Leuconostoc citreum, Leuconostoc falkenbergense, Leuconostoc fallax, Leuconostoc garlicum, Leuconostoc gelidum, Leuconostoc holzapfelii, Leuconostoc inhae, Leuconostoc kimchii, Leuconostoc lactis, Leuconostoc kitchii, Leuconostoc mesenteroides, Leuconostoc miyukkimchii, Leuconostoc palmae, Leuconostoc pseudomesenteroides, Leuconostoc rapi, Leuconostoc suionicum, or a combination thereof.

10. The genetically modified probiotic bacterium of any of embodiments 2-9, wherein the lactic acid bacterium belongs to the genus Oenococcus and include Oenococcus alcoholitolerans, Oenococcus kitaharae, Oenococcus oeni, Oenococcus sicerae, or a combination thereof. 11. The genetically modified probiotic bacterium of any of embodiments 2-10, wherein the lactic acid bacterium belongs to the genus Pediococcus and include Pediococcus acidilactici, Pediococcus argentinicus, Pediococcus cellicola, Pediococcus claussenii, Pediococcus damnosus, Pediococcus ethanolidurans, Pediococcus inopinatus, Pediococcus parvulus, Pediococcus pentosaceus, Pediococcus perniciosus, Pediococcus siamensis, Pediococcus stilesii, or a combination thereof.

12. The genetically modified probiotic bacterium of any of embodiments 2-11, wherein the lactic acid bacterium belongs to the genus Propionibacterium and include Propionibacterium freudenreichii.

13. The genetically modified probiotic bacterium of any of embodiments 2-12, wherein the lactic acid bacterium belongs to the genus Streptococcus and include Streptococcus acidominimus, Streptococcus agalactiae, Streptococcus alactolyticus, Streptococcus anginosus, Streptococcus australis, Streptococcus caballi, Streptococcus cameli, Streptococcus canis, Streptococcus caprae, Streptococcus castoreus, Streptococcus cricetid, Streptococcus constellatus, Streptococcus cristatus, Streptococcus cuniculi, Streptococcus danieliae, Streptococcus dentasini, Streptococcus dentiloxodontae, Streptococcus dentirousetti, Streptococcus devriesei, Streptococcus didelphis, Streptococcus downei, Streptococcus dysgalactiae, Streptococcus entericus, Streptococcus equi, Streptococcus equinus, Streptococcus ferus, Streptococcus gallinaceus, Streptococcus gallolyticus, Streptococcus gordonii, Streptococcus halichoeri, Streptococcus halotolerans, Streptococcus henryi, Streptococcus himalayensis, Streptococcus hongkongensis, Streptococcus hyointestinalis, Streptococcus hyovaginalis, Streptococcus ictalurid, Streptococcus infantarius, Streptococcus infantis, Streptococcus iniae, Streptococcus intermedius, Streptococcus lactarius, Streptococcus loxodontisalivarius, Streptococcus lutetiensis, Streptococcus macacae, Streptococcus marimammalium, Streptococcus marmotae, Streptococcus massiliensis, Streptococcus merionis, Streptococcus minor, Streptococcus mitis, Streptococcus moroccensis, Streptococcus mutans, Streptococcus oralis, Streptococcus oricebi, Streptococcus oriloxodontae, Streptococcus orisasini, Streptococcus orisratti, Streptococcus orisuis, Streptococcus ovis, Streptococcus panodentis, Streptococcus pantholopis, Streptococcus parasanguinis, Streptococcus parasuis, Streptococcus parauberis, Streptococcus peroris, Streptococcus pharynges, Streptococcus phocae, Streptococcus pluranimalium, Streptococcus plurextorum, Streptococcus pneumoniae, Streptococcus porci, Streptococcus porcinus, Streptococcus porcorum, Streptococcus pseudopneumoniae, Streptococcus pseudoporcinus, Streptococcus pyogenes, Streptococcus ratti, Streptococcus rifensis, Streptococcus rubneri, Streptococcus rupicaprae, Streptococcus salivarius, Streptococcus salivarius subsp. thermophilus, Streptococcus saliviloxodontae, Streptococcus sanguinis, Streptococcus sinensis, Streptococcus sobrinus, Streptococcus suis, Streptococcus tangierensis, Streptococcus thoraltensis, Streptococcus troglodytae, Streptococcus troglodytidis, Streptococcus tigurinus, Streptococcus thermophilus, Streptococcus uberis, Streptococcus urinalis, Streptococcus ursoris, Streptococcus vestibularis, Streptococcus zooepidemicus, or a combination thereof.

14. The genetically modified probiotic bacterium of any of embodiments 2-13, wherein the lactic acid bacterium belongs to the genus Tetragenococcus and include Tetragenococcus halophilus, Tetragenococcus koreensis, Tetragenococcus muriaticus, Tetragenococcus osmophilus, Tetragenococcus solitarius, or a combination thereof.

15. The genetically modified probiotic bacterium of any of embodiments 2-14, wherein the lactic acid bacterium belongs to the genus Vagococcus and include Vagococcus acidifermentans, Vagococcus bubulae, Vagococcus carniphilus, Vagococcus coleopterorum, Vagococcus elongatus, Vagococcus entomophilus, Vagococcus fessus, Vagococcus fluvialis, Vagococcus humatus, Vagococcus hydrophili, Vagococcus lutrae, Vagococcus martis, Vagococcus penaei, Vagococcus salmoninarum, Vagococcus silage, Vagococcus teuberi, Vagococcus vulneris, Vagococcus xieshaowenii, Vagococcus zengguangii, or a combination thereof.

16. The genetically modified probiotic bacterium of any of embodiments 2-15, wherein the lactic acid bacterium belongs to the genus Weissella and include Weissella cibaria, Weissella confusa, Weissella halotolerans, Weissella hellenica, Weissella kandleri, Weissella kimchii, Weissella koreensis, Weissella minor, Weissella paramesenteroides, Weissella soli, Weissella thailandensis, and Weissella viridescens, or a combination thereof.

17. The genetically modified probiotic bacterium of any of embodiments 2-16, wherein the lactic acid bacterium includes Lactococcus lactis.

18. The genetically modified probiotic bacterium of any of embodiments 1-17, wherein the heterologous gene encoding the glutamic acid decarboxylase includes gadB.

19. The genetically modified probiotic bacterium of embodiment 18, wherein the gadB includes Lactococcus lactis gadB having at least 80% sequence identity to a sequence as set forth in SEQ ID NO: 1.

20. The genetically modified probiotic bacterium of embodiment 18 or 19, wherein the gadB includes Lactococcus lactis gadB having an amino acid sequence as set forth in SEQ ID NO: 1.

21. The genetically modified probiotic bacterium of any of embodiments 18-20, wherein the gadB includes Lactococcus lactis gadB having at least 80% sequence identity to a nucleic acid sequence encoding a sequence as set forth in SEQ ID NO: 1.

22. The genetically modified probiotic bacterium of any of embodiments 18-21, wherein the gadB includes Lactococcus lactis gadB having at least 80% sequence identity to a nucleic acid sequence as set forth in SEQ ID NO: 3.

23. The genetically modified probiotic bacterium of any of embodiments 18-22, wherein the gadB includes Lactococcus lactis gadB having a sequence as set forth in SEQ ID NO: 3.

24. The genetically modified probiotic bacterium of any of embodiments 1-23, wherein the heterologous gene encoding the glutamate/GABA antiporter includes gadC.

25. The genetically modified probiotic bacterium of embodiment 24, wherein the gadC includes Lactococcus lactis gadC having at least 80% sequence identity to a sequence as set forth in SEQ ID NO: 2.

26. The genetically modified probiotic bacterium of embodiment 24 or 25, wherein the gadC includes Lactococcus lactis gadC having an amino acid sequence as set forth in SEQ ID NO: 2.

27. The genetically modified probiotic bacterium of any of embodiments 24-26, wherein the gadC includes Lactococcus lactis gadC having at least 80% sequence identity to a nucleic acid sequence encoding a sequence as set forth in SEQ ID NO: 2.

28. The genetically modified probiotic bacterium of any of embodiments 24-27, wherein the gadC includes Lactococcus lactis gadC having at least 80% sequence identity to a nucleic acid sequence as set forth in SEQ ID NO: 4.

29. The genetically modified probiotic bacterium of any of embodiments 24-28, wherein the gadC includes Lactococcus lactis gadC having a sequence as set forth in SEQ ID NO: 4.

30. The genetically modified probiotic bacterium of any of embodiments 1-29, wherein both the heterologous gene encoding a glutamic acid decarboxylase and the heterologous gene encoding a glutamate/GABA antiporter are operably linked to a heterologous promoter.

31. The genetically modified probiotic bacterium of embodiment 30, wherein the heterologous promoter includes an inducible promoter.

32. The genetically modified probiotic bacterium of embodiment 31, wherein the inducible promoter includes PgroES, pL, pR, cspA, pLac, pBad, pTac, Ptrp, PhoA, recA, proU, sct, tetA, cadA, cadR, nar, p170, nisin-inducible promoter, or P_{aguB}.

33. The genetically modified probiotic bacterium of embodiment 30, wherein the heterologous promoter includes a constitutive promoter.

34. The genetically modified probiotic bacterium of embodiment 33, wherein the constitutive promoter includes P1, P2, P3, P4, P5, P6, P7, P8, P32, P45, LacA, PPepN, P6C, P13C, or PTS-IIC.

35. The genetically modified probiotic bacterium of embodiment 33 or 34, wherein the constitutive promoter includes a P2, a P5, or a P8 promoter.

36. The genetically modified probiotic bacterium of embodiment 34 or 35, wherein the P2 promoter has a sequence as set forth in SEQ ID NO: 12; the P5 promoter has a sequence as set forth in SEQ ID NO: 13; and/or the P8 promoter has a sequence as set forth in SEQ ID NO: 5 or SEQ ID NO: 6.

37. The genetically modified probiotic bacterium of any of embodiments 1-29, wherein the heterologous gene encoding a glutamic acid decarboxylase is operably linked to a first heterologous promoter and the heterologous gene encoding a glutamate/GABA antiporter is operably linked to a second heterologous promoter.

38. The genetically modified probiotic bacterium of embodiment 37, wherein the first heterologous promoter and/or the second heterologous promoter includes an inducible promoter.

39. The genetically modified probiotic bacterium of embodiment 38, wherein the inducible promoter includes PgroES, pL, pR, cspA, pLac, pBad, pTac, Ptrp, PhoA, recA, proU, sct, tetA, cadA, cadR, nar, p170, nisin-inducible promoter, or P_{aguB}.

40. The genetically modified probiotic bacterium of embodiment 37, wherein the first heterologous promoter and/or the second heterologous promoter includes a constitutive promoter.

41. The genetically modified probiotic bacterium of embodiment 40, wherein the constitutive promoter includes P1, P2, P3, P4, P5, P6, P7, P8, P32, P45, LacA, PPepN, P6C, P13C, or PTS-IIC.

42. The genetically modified probiotic bacterium of embodiment 40, wherein the constitutive promoter includes a P2, a P5, or a P8 promoter.

43. The genetically modified probiotic bacterium of embodiment 41 or 42, wherein the P2 promoter has a sequence as set forth in SEQ ID NO: 12; the P5 promoter has a sequence as set forth in SEQ ID NO: 13; and/or the P8 promoter has a sequence as set forth in SEQ ID NO: 5 or SEQ ID NO: 6.

44. The genetically modified probiotic bacterium of any of embodiments 1-43, wherein the genetically modified probiotic bacterium includes endogenous glutamic acid decarboxylase and glutamate/GABA antiporter genes.

45. The genetically modified probiotic bacterium of any of embodiments 1-44, wherein the heterologous gene encoding the glutamic acid decarboxylase and the heterologous gene encoding the glutamate/GABA antiporter are part of an expression cassette of a genetic construct.

46. The genetically modified probiotic bacterium of embodiment 45, wherein the genetic construct is not integrated in the genome of the genetically modified probiotic bacterium.

47. The genetically modified probiotic bacterium of embodiment 45, wherein the genetic construct is integrated into the genome of the genetically modified probiotic bacterium.

48. The genetically modified probiotic bacterium of embodiment 47, wherein the integrated genetic construct disrupts an endogenous gene.

49. The genetically modified probiotic bacterium of embodiment 48, wherein the endogenous gene is leuA.

50. The genetically modified probiotic bacterium of any of embodiments 45-49, wherein the genetic construct further includes an upstream homology arm and a downstream homology arm.

51. The genetically modified probiotic bacterium of embodiment 50, wherein the upstream and downstream homology arms include sequences homologous to an endogenous gene.

52. The genetically modified probiotic bacterium of embodiment 51, wherein the probiotic bacteria is L. lactis and the endogenous gene is leuA.

53. The genetically modified probiotic bacterium of any of embodiments 50-52, wherein the upstream homology arm has a sequence having at least 90% sequence identity to the sequence as set forth in SEQ ID NO: 9.

54. The genetically modified probiotic bacterium of any of embodiments 50-53, wherein the upstream homology arm has a sequence as set forth in SEQ ID NO: 9.

55. The genetically modified probiotic bacterium of any of embodiments 50-54, wherein the downstream homology arm has a sequence having at least 90% sequence identity to the sequence as set forth in SEQ ID NO: 10.

56. The genetically modified probiotic bacterium of any of embodiments 50-55, wherein the downstream homology arm has a sequence as set forth in SEQ ID NO: 10.

57. The genetically modified probiotic bacterium of any of embodiments 51-56, wherein transcription of the heterologous gene encoding the glutamic acid decarboxylase and transcription of the heterologous gene encoding the glutamate/GABA antiporter is in the opposite direction from transcription of the endogenous gene.

58. The genetically modified probiotic bacterium of any of embodiments 45-57, wherein the genetic construct includes a sequence as set forth in SEQ ID NO: 11.

59. The genetically modified probiotic bacterium of any of embodiments 45-58, wherein the genetic construct further includes a selectable marker.

60. The genetically modified probiotic bacterium of embodiment 59, wherein the selectable marker confers antibiotic resistance, complements an essential gene, confers chemical resistance, and/or includes a visual marker.

61. The genetically modified probiotic bacterium of embodiment 60, wherein the antibiotic resistance includes erythromycin resistance.

62. The genetically modified probiotic bacterium of any of embodiments 1-61, wherein the genetically modified probiotic bacterium produces an amount of gamma-aminobutyric acid (GABA) that is 2-fold to 200-fold greater as compared to an amount of GABA produced by a control.

63. The genetically modified probiotic bacterium of embodiment 62, wherein the control is probiotic bacteria of the same genus or species that have not been genetically modified.

64. The genetically modified probiotic bacterium of embodiment 62, wherein the control is probiotic bacteria of the same genus or species that have been genetically modified with a control plasmid.

65. The genetically modified probiotic bacterium of any of embodiments 1-64, wherein the genetically modified probiotic bacterium produces 500 ng/ml GABA to 60,000 ng/mL GABA.

66. A genetic construct including:
a promoter operably linked to:
   a gene encoding a glutamic acid decarboxylase; and
   a gene encoding a glutamate/GABA antiporter.

67. The genetic construct of embodiment 66, wherein the promoter is a constitutive promoter or an inducible promoter.

68. The genetic construct of embodiment 67, wherein the constitutive promoter includes P1, P2, P3, P4, P5, P6, P7, P8, P32, P45, LacA, PPepN, P6C, P13C, or PTS-IIC.

69. The genetic construct of embodiment 67, wherein the constitutive promoter includes a P2, a P5, or a P8 promoter.

70. The genetic construct of embodiment 68 or 69, wherein the P2 promoter has a sequence as set forth in SEQ ID NO: 12; the P5 promoter has a sequence as set forth in SEQ ID NO: 13; and/or the P8 promoter has a sequence as set forth in SEQ ID NO: 5 or SEQ ID NO: 6.

71. The genetic construct of embodiment 67, wherein the inducible promoter includes PgroES, pL, pR, cspA, pLac, pBad, pTac, Ptrp, PhoA, recA, proU, sct, tetA, cadA, cadR, nar, p170, nisin-inducible promoter, or P_{aguB}.

72. The genetic construct of any of embodiments 66-71, wherein the gene encoding the glutamic acid decarboxylase includes gadB.

73. The genetic construct of embodiment 72, wherein the gadB includes Lactococcus lactis gadB having at least 80% sequence identity to a sequence as set forth in SEQ ID NO: 1.

74. The genetic construct of embodiment 72 or 73, wherein the gadB includes Lactococcus lactis gadB having an amino acid sequence as set forth in SEQ ID NO: 1.

75. The genetic construct of any of embodiments 72-74, wherein the gadB includes Lactococcus lactis gadB having at least 80% sequence identity to a nucleic acid sequence encoding a sequence as set forth in SEQ ID NO: 1.

76. The genetic construct of any of embodiments 72-75, wherein the gadB includes Lactococcus lactis gadB having at least 80% sequence identity to a nucleic acid sequence as set forth in SEQ ID NO: 3.

77. The genetic construct of any of embodiments 72-76, wherein the gadB includes Lactococcus lactis gadB having a sequence as set forth in SEQ ID NO: 3.

78. The genetic construct of any of embodiments 72-77, wherein the gene encoding the glutamate/GABA antiporter includes gadC.

79. The genetic construct of embodiment 78, wherein the gadC includes Lactococcus lactis gadC having at least 80% sequence identity to a sequence as set forth in SEQ ID NO: 2.

80. The genetic construct of embodiment 78 or 79, wherein the gadC includes Lactococcus lactis gadC having an amino acid sequence as set forth in SEQ ID NO: 2.

81. The genetic construct of any of embodiments 78-80, wherein the gadC includes Lactococcus lactis gadC having at least 80% sequence identity to a nucleic acid sequence encoding a sequence as set forth in SEQ ID NO: 2.

82. The genetic construct of any of embodiments 78-81, wherein the gadC includes Lactococcus lactis gadC having at least 80% sequence identity to a nucleic acid sequence as set forth in SEQ ID NO: 4.

83. The genetic construct of any of embodiments 78-82, wherein the gadC includes Lactococcus lactis gadC having a sequence as set forth in SEQ ID NO: 4.

84. The genetic construct of any of embodiments 66-83, wherein the genetic construct further includes a selectable marker.

85. The genetic construct of embodiment 84, wherein the selectable marker confers antibiotic resistance, complements an essential gene, confers chemical resistance, and/or includes a visual marker.

86. The genetic construct of embodiment 85, wherein the antibiotic resistance includes erythromycin resistance.

87. The genetic construct of any of embodiments 66-86, wherein the promoter and the genes are part of an expression cassette.

88. The genetic construct of embodiment 87, wherein the genetic construct further includes an upstream homology arm and a downstream homology arm flanking the expression cassette that are homologous to a gene in a probiotic bacterium.

89. The genetic construct of embodiment 88, wherein the probiotic bacterium is L. lactis.

90. The genetic construct of embodiment 88 or 89, wherein the gene in the probiotic bacterium includes leuA.

91. The genetic construct of any of embodiments 88-90, wherein the upstream homology arm has a sequence having at least 90% sequence identity to the sequence as set forth in SEQ ID NO: 9.

92. The genetic construct of any of embodiments 88-91, wherein the upstream homology arm has a sequence as set forth in SEQ ID NO: 9.

93. The genetic construct of any of embodiments 88-92, wherein the downstream homology arm has a sequence having at least 90% sequence identity to the sequence as set forth in SEQ ID NO: 10.

94. The genetic construct of any of embodiments 88-93, wherein the downstream homology arm has a sequence as set forth in SEQ ID NO: 10.

95. The genetic construct of any of embodiments 88-94, wherein the gene encoding the glutamic acid decarboxylase and the gene encoding the glutamate/GABA antiporter are transcribed in an opposite direction from transcription of the gene in the probiotic bacterium.

96. The genetic construct of any of embodiments 66-95, wherein the genetic construct includes a sequence as set forth in SEQ ID NO: 11.

97. The genetic construct of any of embodiments 66-96, wherein the genetic construct includes 5′ to 3′: the promoter, the gene encoding a glutamate/GABA antiporter, and the gene encoding a glutamic acid decarboxylase.

98. A method of preparing genetically modified probiotic bacteria, including introducing a genetic construct of any of embodiments 66-97 into probiotic bacteria to obtain genetically modified probiotic bacteria; and culturing the genetically modified probiotic bacteria in media. 99. The method of embodiment 98, wherein the culturing includes growing the genetically modified probiotic bacteria at 20° C. to 50° C.

100. The method of embodiment 98 or 99, wherein the culturing further includes adding glutamic acid HCl to the media.

101. The method of any of embodiments 98-100, wherein the culturing includes growing the genetically modified probiotic bacteria to an OD600 of 0.5 to 2.

102. A method of producing gamma-aminobutyric acid (GABA), including culturing the genetically modified probiotic bacterium of any of embodiments 1-65 in media.

103. A composition including the genetically modified probiotic bacterium of any of embodiments 1-65 and a pharmaceutically acceptable carrier.

104. The composition of embodiment 103, wherein the composition includes an oral composition.

105. The composition of embodiment 103 or 104, wherein the composition includes a solid or a liquid.

106. The composition of embodiment 105, wherein the solid includes a lyophilized powder.

107. The composition of embodiment 106, wherein the lyophilized powder is dispersed in a liquid.

108. The composition of any of embodiments 105-107, wherein the liquid includes a liquid suspension.

109. The composition of any of embodiments 105-108, wherein the liquid includes water or juice.

110. The composition of any of embodiments 103-109, wherein the composition is part of a dairy product.

111. The composition of embodiment 110, wherein the dairy product includes yogurt, milk, cheese, kefir, ice cream, and butter.

112. The composition of any of embodiments 103-111, wherein the composition further includes a prebiotic.

113. The composition of embodiment 112, wherein the prebiotic includes fiber, amino acids, oligosaccharides, or polyamines.

114. A method of treating a disease or disorder in a subject in need thereof including administering a therapeutically effective amount of the composition of any of embodiments 103-113.

115. The method of embodiment 114, wherein the disease or disorder is associated with GABA deficiency, impaired GABA receptor mediated signaling, and/or an excess of excitatory neurotransmitters.

116. The method of embodiment 115, wherein the disease or disorder associated with GABA deficiency, impaired GABA receptor mediated signaling, and/or an excess of excitatory neurotransmitters includes anxiety, autism, multiple sclerosis, schizophrenia, Parkinson's Disease, Huntington's disease, epilepsy, post-traumatic stress disorder (PTSD), and stroke and its complications.

117. The method of any of embodiments 114-116, wherein the disease or disorder is an inflammatory autoimmune disease.

118. The method of embodiment 117, wherein the inflammatory autoimmune disease includes cachexia, inflammatory bowel diseases (IBD), psoriatic arthritis, rheumatoid arthritis, Type 1 diabetes, Type 2 Diabetes, Sjögren's syndrome, systemic lupus erythematosus, celiac disease, Graves' disease, Hashimoto's thyroiditis, Addison's disease, dermatomyositis, psoriasis, chronic inflammatory demyelinating polyneuropathy, Guillain-Barre syndrome, myasthenia gravis, and vasculitis.

119. The method of embodiment 118, wherein the IBD is Crohn's disease or ulcerative colitis.

120. A genetic construct including:
a constitutive promoter operably linked to:
   a gene encoding a glutamic acid decarboxylase; and
   a gene encoding a glutamate/GABA antiporter.

121. The genetic construct of embodiment 120, wherein the constitutive promoter includes a P2, a P5, or a P8 promoter.

122. The genetic construct of embodiment 121, wherein the P2 promoter has a sequence as set forth in SEQ ID NO: 12; the P5 promoter has a sequence as set forth in SEQ ID NO: 13; or the P8 promoter has a sequence as set forth in SEQ ID NO: 5 or SEQ ID NO: 6.

123. The genetic construct of any of embodiments 120-122, wherein the gene encoding the glutamic acid decarboxylase includes gadB.

124. The genetic construct of embodiment 123, wherein the gadB includes Lactococcus lactis gadB having at least 80% sequence identity to a sequence as set forth in SEQ ID NO: 1.

125. The genetic construct of any of embodiments 120-124, wherein the gene encoding the glutamate/GABA antiporter includes gadC.

126. The genetic construct of embodiment 125, wherein the gadC includes Lactococcus lactis gadC having at least 80% sequence identity to a sequence as set forth in SEQ ID NO: 2.

127. The genetic construct of any of embodiments 120-126, wherein the genetic construct further includes a selectable marker.

128. The genetic construct of embodiment 127, wherein the selectable marker confers erythromycin resistance.

129. The genetic construct of any of embodiments 120-128, wherein the promoter and the genes are part of an expression cassette.

130. The genetic construct of any of embodiments 120-129, wherein the genetic construct further includes an upstream homology arm and a downstream homology arm flanking the expression cassette that are homologous to a gene in a probiotic bacterium.

131. The genetic construct of embodiment 130, wherein the upstream homology arm has a sequence having at least 90% sequence identity to the sequence as set forth in SEQ ID NO: 9.

132. The genetic construct of embodiment 130 or 131, wherein the downstream homology arm has a sequence having at least 90% sequence identity to the sequence as set forth in SEQ ID NO: 10.

133. The genetic construct of any of embodiments 120-132, wherein the genetic construct includes a sequence as set forth in SEQ ID NO: 11.

134. The genetic construct of any of embodiments 120-133, wherein the genetic construct includes 5′ to 3′: the promoter, the gene encoding a glutamate/GABA antiporter, and the gene encoding a glutamic acid decarboxylase.

135. A cell including a genetic construct of any one of embodiments 120-134.

136. The cell of embodiment 135, wherein the cell includes a probiotic bacterium. 137. The cell of embodiment 136, wherein the probiotic bacterium includes Lactococcus lactis. 138. A method of producing gamma-aminobutyric acid (GABA), including culturing the probiotic bacterium of embodiment 136 or 137 in media.

139. A method of preparing genetically modified probiotic bacteria that produce gamma-aminobutyric acid (GABA), including introducing a genetic construct of any one of embodiments 120-134 into probiotic bacteria to obtain genetically modified probiotic bacteria; and culturing the genetically modified probiotic bacteria in media.

140. The method of embodiment 139, wherein the culturing includes growing the genetically modified probiotic bacteria at 20° C. to 50° C.

141. The method of embodiment 139 or 140, wherein the culturing further includes adding glutamic acid HCl to the media.

142. The method of any of embodiments 138-141, wherein the culturing includes growing the genetically modified probiotic bacteria to an OD600 of 0.5 to 2.

143. The method of any of embodiments 138-142, wherein the genetically modified probiotic bacteria produces an amount of GABA that is 2-fold to 200-fold greater as compared to an amount of GABA produced by a control.

144. The method of embodiment 143, wherein the control is probiotic bacteria of the same genus or species that have not been genetically modified or that have been genetically modified with a control plasmid.

145. The method of any of embodiments 138-144, wherein the genetically modified probiotic bacteria produce 500 ng/mL GABA to 60,000 ng/ml GABA.

146. A composition including the probiotic bacteria of embodiment 136 or 137 and a pharmaceutically acceptable carrier.

147. The composition of embodiment 146, wherein the composition includes an oral composition.

148. The composition of embodiment 146 or 147, wherein the composition includes a solid or a liquid.

149. The composition of embodiment 148, wherein the solid includes a lyophilized powder. 150. The composition of any of embodiments 146-149, wherein the composition is part of a dairy product.

151. The composition of embodiment 150, wherein the dairy product includes yogurt, milk, cheese, kefir, ice cream, or butter.

152. A method of treating a disease or disorder in a subject in need thereof including administering a therapeutically effective amount of the composition of any of embodiments 146-151.

153. The method of embodiment 152, wherein the disease or disorder is associated with GABA deficiency, impaired GABA receptor mediated signaling, and/or an excess of excitatory neurotransmitters.

154. The method of embodiment 153, wherein the disease or disorder associated with GABA deficiency, impaired GABA receptor mediated signaling, and/or an excess of excitatory neurotransmitters includes alcoholism, depression, anxiety, autism, multiple sclerosis, schizophrenia, Parkinson's Disease, Huntington's disease, epilepsy, post-traumatic stress disorder (PTSD), or stroke and its complications.

155. The method of any of embodiments 152-154, wherein the disease or disorder is associated with inflammation.

156. The method of any of embodiments 152-155, wherein the disease or disorder is an inflammatory autoimmune disease.

157. The method of embodiment 156, wherein the inflammatory autoimmune disease includes cachexia, inflammatory bowel diseases (IBD), psoriatic arthritis, rheumatoid arthritis, Type 1 diabetes, Type 2 Diabetes, Sjögren's syndrome, systemic lupus erythematosus, celiac disease, Graves' disease, Hashimoto's thyroiditis, Addison's disease, dermatomyositis, psoriasis, chronic inflammatory demyelinating polyneuropathy, Guillain-Barre syndrome, myasthenia gravis, or vasculitis.

158. The method of embodiment 157, wherein the IBD is Crohn's disease or ulcerative colitis.

### EXPERIMENTAL EXAMPLES

### Example 1

Constructs of GAD L. Lactis produce enhanced levels of GABA. This example demonstrated that L. Lactis having an additional copy of each of the gadB gene and gadC gene produced enhanced levels of GABA as compared to a control.

The L. lactis gadB and gadC genes encode glutamic acid decarboxylase (GAD) and a glutamate-GABA antiporter (GadC), respectively. To maximize GABA production, an additional copy of each of the gadB gene and gadC gene from L. lactis strain IL1403 were added to an L. lactis strain IL 1403 that naturally has an endogenous copy of the gadB gene and an endogenous copy of the gadC gene and produces GABA. The complete genome of Lactococcus lactis subsp. Lactis IL 1403 is found at GenBank accession number AE005176.1. The additional copies of gadB and gadC (genomic position 1326770-1323662, 3109 bp) were cloned downstream of shortened versions of constitutive promoters, P2, P5 and P8 (Zhu et al. FEMS Microbiol Lett 2015, 362). A short version of the P8 promoter (SEQ ID NO: 5), P8s, was used for the P8-gadCB construct. The ribosome binding sites of the promoters were included. The P2-gadCB, P5-gadCB, and P8s-gadCB constructs were then cloned antisense and within the upstream (genomic position 1239855-1240223, 369 bp) and downstream (genomic position 1241340-1241054, 287 bp) regions of the L. lactis strain IL1403 leuA gene. Each construct was then cloned into the Pstl site of an L. lactis plasmid, pGh9: ISS1 (Thibessard et al. Can. J. Microbiol. 2002, 48:473-478) or pBVGh (Blancato and Magni. Lett. Appl. Microbiol. 2010, 50, 542-546) and transformed into L. lactis strain IL1403 (Blancato and Magni. Lett. Appl. Microbiol. 2010, 50, 542-546). The resulting strains were named: P2-GAD-L. lactis, P5-GAD-L. lactis and P8s-GAD-L. lactis.

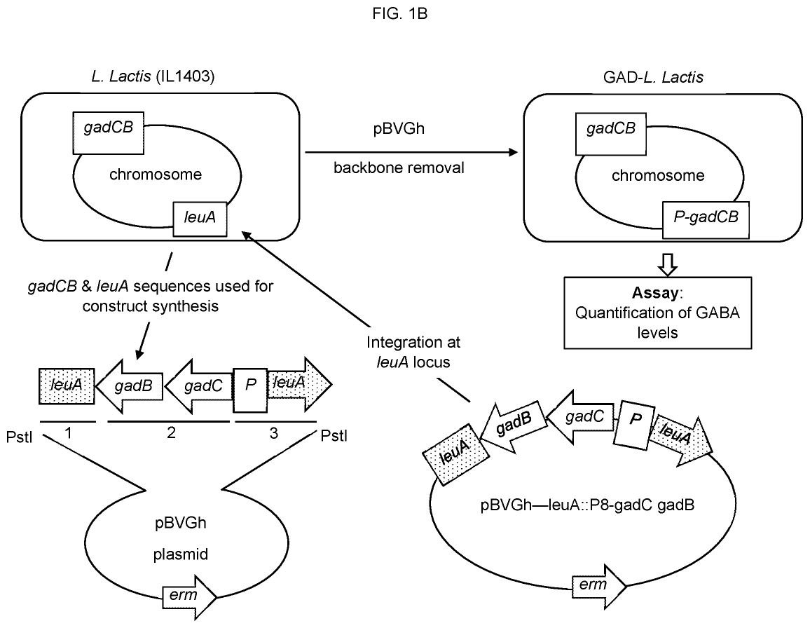

To test whether the newly constructed L. lactis strains produced increased levels of GABA, P2-GAD-L. lactis, P5-GAD-L. lactis and P8s-GAD-L. lactis (constructs in pGh9: ISS1 vector) were cultured in GM17+erythromycin medium until the cultures reached absorbances (OD600) of 0.5, 1, 1.5 and 2. After culture, samples were centrifuged and supernatants were transferred to new tubes and kept at −80° C. The levels of GABA released by the bacteria to the supernatants were analyzed by enzyme-linked immunosorbent assay (ELISA) (Rocky Mountain Diagnostics; Catalog #: BA E-2500R) and corrected to the colony forming units present in each culture (FIG. 2).

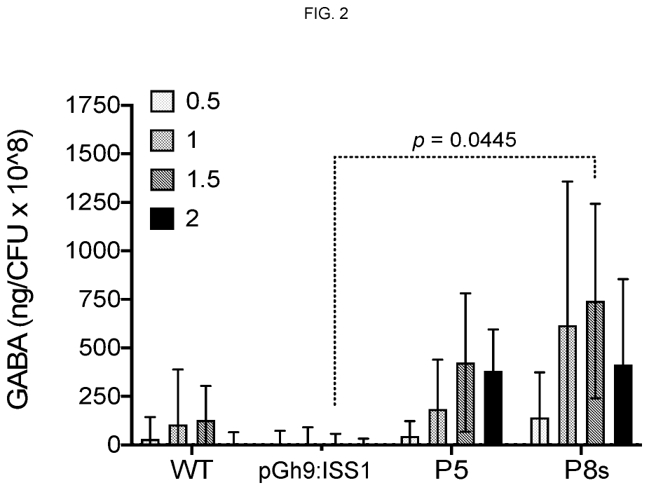

As shown in FIG. 2, L. lactis with the integrated P8 construct produced GABA levels that were significantly higher than those produced by L. lactis with pGh9: ISS1, an unmodified plasmid vector control (plasmid that was used to generate constructs P5 and P8s when inserting GAD genes). Although P5 also produced increased levels of GABA as compared to the plasmid vector control or wild type (WT), the statistical analysis did not show any significance when compared to the pGh9: ISS1 control. GABA production was highest during mid-log phase, OD600 1.5. Based on these results, the P8s construct was selected for further analysis in vivo (FIGS. 3-5).

### Example 2

Oral treatment with GAD L. Lactis reduces the severity of experimental autoimmune encephalomyelitis (EAE), an animal model of Multiple Sclerosis (MS). This Example demonstrated that treatment of mice with P8-GAD-L. lactis, a genetically engineered L. lactis with enhanced GABA production, is protective against EAE induced in the mice.

MS is a disease that involves a complex interaction between the central nervous system and the peripheral immune system. In order to study the physiological relevance of different therapies that will help understand and treat this disease, the use of a mouse model (EAE) is desirable, as in vitro experiments can only give a limited and partial understanding of the immune response. Mice are used because they are genetically in-bred and for the studies described herein, genetic identity between mice is essential. EAE can be induced passively by adoptive transfer of autoreactive T cells or actively by subcutaneous injection of self-antigens obtained from neuronal myelin homogenates: proteolipid protein (PLP), myelin basic protein (MBP), myelin oligodendrocyte glycoprotein (MOG), or peptides corresponding to the encephalitogenic portions of these proteins. In mice, induction requires the emulsification of one of the self-antigens in complete Freund's adjuvant and additional intraperitoneal or intravenous administration of pertussis toxin. C57BL/6 mice were used to study EAE using a commercial EAE induction kit (Hooke labs, kit EK-2110). The protocol for the induction and monitorization of the disease has been described (Stromnes and Goverman. Nature Protocols 2006, 1, 1810-1819). Mice are induced with EAE by subcutaneous injection of an emulsion of 250 μg of MOG in complete Freund's adjuvant on the flanks at 2 sites. The same day and 24 hours later the mice receive an IP injection of 200 ng of Pertussis Toxin (200 μL). Immunization with synthetic MOG35-55, minor components of CNS myelin, produce a progressive neurological disease with extensive plaque-like demyelination, common to the manifestations of multiple sclerosis. EAE mice are scored as described previously (Stromnes and Goverman. Nature Protocols 2006, 1, 1810-1819): 0—no detectable signs of EAE, 0.5—distal limp tail, 1.0—complete limp tail, 1.5—limp tail and hind limb weakness, 2.0—unilateral partial hind limb paralysis, 2.5—bilateral partial hind limb paralysis, 3.0—complete bilateral hind limb paralysis, 3.5—complete bilateral hind limb paralysis and partial front limb paralysis, 4.0—quadriplegia. Those mice that show clinical scores>3.0 for two consecutive days are euthanized due to humane reasons and per IACUC guidelines.

The C57BL/6 EAE model was used to test whether the oral administration of a L. lactis strain capable of producing enhanced levels of GABA would protect mice from a severe form of disease (FIG. 3). The mice were treated by oral gavages with 5×10⁸ bacterial colony forming units (CFU)/day from day 0 to the end of the experiment (day 25), five times per week (Robert et al. Diabetes 2014, 63, 2876-2887). The effects that P8s-GAD-L. lactis had on the progression and severity of EAE and body weight changes were compared with those of the treatment with medium alone (sham) and pGh9: ISS1 empty vector control. The P8s construct was selected based on the statistically significant increase in GABA production observed in in vitro cultures of different constructs at different bacterial growth stages (FIG. 2). pGh9: ISS1-L. lactis was used as a control treatment (L. lactis containing an unmodified pGh9: ISS1 plasmid) as it is unable to produce more GABA than under its genetic and physiological threshold. As a sham control, mice received sterile M17 medium (used to grow L. lactis). The results shown in FIG. 3 demonstrate that 5 treatments per week with P8s-GAD-L. lactis reduced significantly the severity of the disease, as observed by the analysis performed in the disease progression curve (FIG. 3). Mice treated with P8s-GAD-L. lactis had a significantly reduced EAE clinical score averages profile than those treated with medium alone (p<0.001), or treated with pGh9: ISS1-L. lactis (L. lactis having pGh9: ISS1 empty vector) (p<0.001). By contrast, the treatment with pGh9: ISS1-L. lactis did not affect the progression of EAE when compared with the treatment with medium alone. No significant effect of the treatment on EAE disease onset was observed. Thus, the results indicate that treatment with P8s-GAD-L. lactis, a genetically engineered L. lactis with enhanced GABA production, is protective against EAE induced in mice.

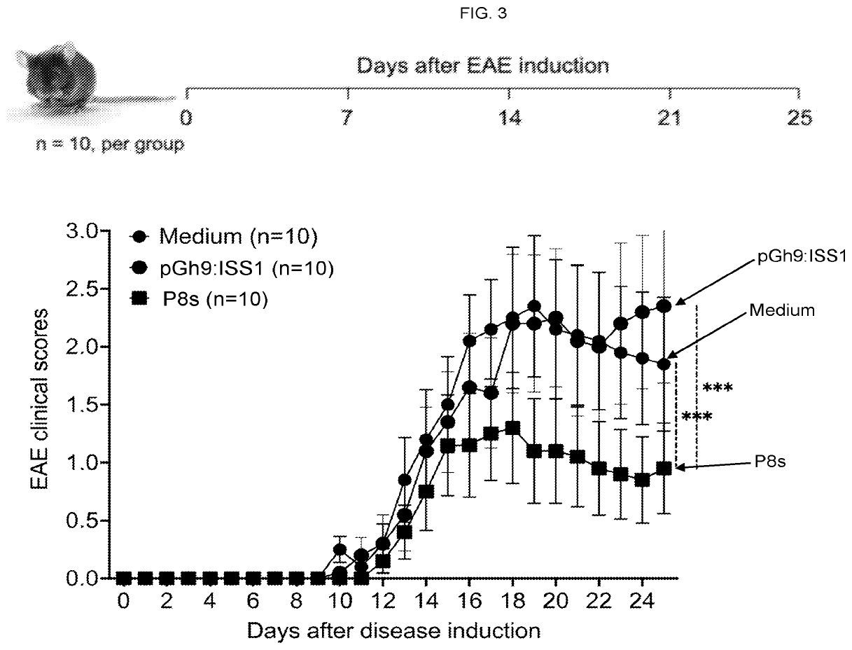

In addition, the treatment with P8s-GAD-L. lactis resulted in differences in the distribution of clinical scores when compared with the treatment with medium alone or treatment with pGh9: ISS1-L. lactis (FIG. 4). P8s-GAD-L. lactis-treated mice developed a milder form of the disease. More than 50% of P8s-GAD-L. lactis-treated mice suffered a mild form of EAE (0-1 in FIG. 4), observed at the peak of the disease (day 19) and at the end of the experiment (day 25). By contrast, those mice treated with medium or pGh9: ISS1-L. lactis suffered a more severe form of disease, with >50% mice suffering severe clinical scores (1.5-2, 2.5-3, and 3.5-5 in FIG. 4).

### Example 3

Oral treatment with GAD L. Lactis prevents body weight loss during EAE, and animal model of Multiple Sclerosis. This Example demonstrated that treatment of mice with P8-GAD-L. lactis, a genetically engineered L. lactis with enhanced GABA production, reduced body weight loss associated with EAE induced in mice.

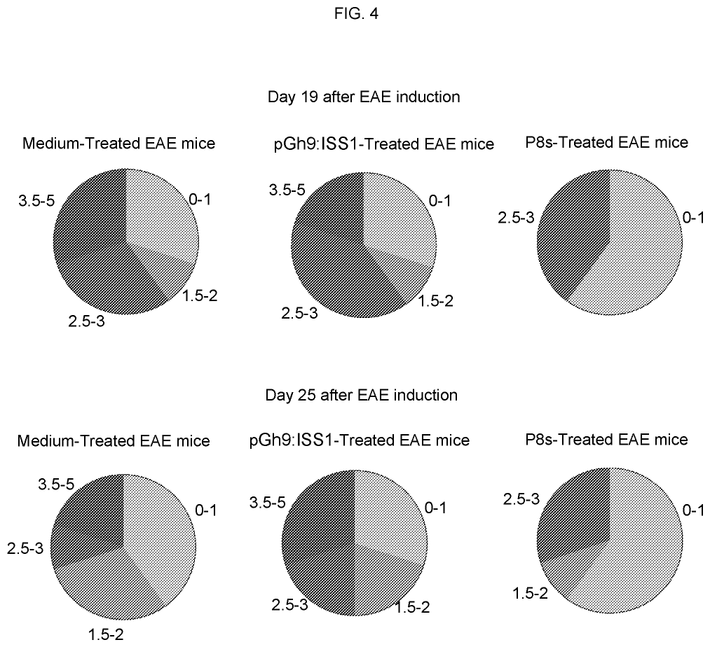

Loss of body weight is directly associated with the progression of EAE disease in mice. The effects of the treatment with medium, pGh9: ISS1-L. lactis, and P8-GAD-L. lactis on body weight changes were compared during the experiment. The body weights of all mice were taken every 3-4 days and compared statistically (FIG. 5). The changes of the body weights observed (versus initial weights taken on day 0) indicated a correlation with disease progression. Those animals with highest clinical scores suffered a more profound loss of body weight. As a result, the animals treated with P8-GAD-L. lactis lost less body weight than those mice treated with pGh9: ISS1-L. lactis (p<0.05) and medium (p<0.01). pGh9: ISS1-L. lactis treatment also reduced the loss of body weights when compared with medium (p<0.01). In conclusion, the results indicate that the oral treatment with L. lactis reduced the effects that EAE disease has on body weight loss, and that the overproduction of GABA prevents further loss of body weight during disease.

### Example 4

Expression of gadB conferred by constitutive expression. This Example demonstrated that gadB expression is significantly higher in an L. lactis bacteria genetically modified to have a construct including a gadB gene and a gadC gene operably linked to a constitutive P8 promoter.

Total RNA was isolated from L lactis strains (L. lactis unmodified (WT), pGh9: ISS1-L. lactis (P), pGh-P2-pGh-GAD-L. lactis (P2), pGh-P5-GAD-L. lactis (P5) and pGh-P8s-GAD-L. lactis (P8). Reverse transcription and qPCR were carried out using the ReverTra Ace™ qPCR RT Master Mix with gDNA Remover (Toyobo) and THUNDERBIRD™ Next SYBR™ qPCR Mix (Toyobo), respectively. Relative expression of gadB was determined using 16s rRNA as a reference gene. The ΔΔCt was calculated by comparing the ΔCt of L. lactis with P, P2, P5 or P8 to the ΔCt of the WT strain. The fold-change in expression was calculated as 2^{-ΔΔCt}. An ANOVA (P=0.031) and Dunnett's multiple comparison test were used to compare means. gadB expression is significantly higher in P8s than in the WT strain (P=0.0325).

### Example 5

GABA produced by L. lactis strains. This Example demonstrated that GABA production increased over time in P8s-GAD-L. lactis cultured in glutamic acid-HCl.

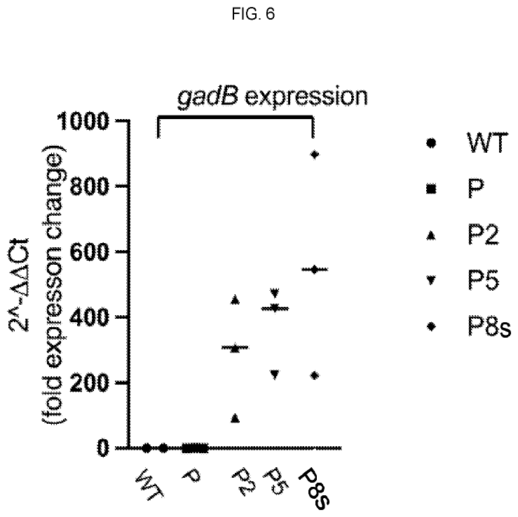

A GABA ELISA (LDN®, Nordhorn, Germany) was used to measure GABA levels in L. lactis strain supernatants (pGh9: ISS1-L. lactis (P) and pGh-P8s-GAD-L. lactis (P8). Strains were cultured in GM17+erm alone (0) or with glutamic acid HCl (50 mM (1), 150 mM (2) or 200 mM (3)) and incubated at 30° C. for 3 hours or the indicated times. GABA concentration was expressed as GABA concentration of the sample minus the GABA concentration of the media control, normalized for CFU/mL. All P8 strains cultured with glutamic acid-HCl produced significantly more GABA than the P8 strain cultured in M17+erm (1, P=0.002; 2 and 3, P<0.001) or P strains cultured with or without glutamic acid-HCl (P_0 to P8_1, P=0.0017; P8_2 and 3, P<0.001; P_1 to P8_1, P=0.0042, P_1 to P8_2 and 3, P<0.001; P_2 to P8_1, P=0.0037, P_2 to P8_2 and 3, P<0.001; P_3 to P8_1, P=0.0038, P_3 to P8_2 and 3, P<0.001) (FIG. 7). The ANOVA was P<0.001 and the group means were analyzed by Tukey's multiple comparison. GABA production increased over time in P8 cultured in 200 mM glutamic acid-HCl.

### Example 6. Materials and Methods

Plasmids, strains, media and culture conditions. Plasmid pGh9: ISS1 (Maguin et al. J. Bacteriol. 1996, 178, 931-935) harbors the pG+ (host) (derivative of pWV01) thermosensitive replicon and the erythromycin (erm) resistance gene. This plasmid replicates in Escherichia coli and Lactococcus lactis at the permissive temperature (30° C.) but is unstable above 37° C. Escherichia coli strain MC1061 (Lucigen), a recA positive strain, was used for propagating all pGh9: ISS1 plasmid constructs. Luria burtani (LB) broth and agar with 200 μg/mL erm was used to culture E. coli containing plasmids used in this study. All culturing of E. coli was done with aeration (200 rpm). The Lactococcus lactis strain IL1403 (Bolotin et al. Genome Res. 2001, 11, 731-753) was manipulated in this study and cultured in GM17 (M17 with 0.5% glucose) broth or agar media with 5 μg/mL erm for plasmid selection. Culture of L. lactis was done without aeration at 30° C. unless otherwise specified. The plasmid pBVGh (Blancato and Magni, Appl. Microbiol. 2010, 50, 542-546), a modified version of pGh9: ISS1, is used to facilitate integration of the Px-GAD constructs into the leuA locus of the L. lactis chromosome. This plasmid lacks the ISS1 transposon for random chromosomal integration and contains the bfaB gene (encodes β-galactosidase) that allows for blue-white screening.

Engineering pPx-GAD plasmids. Constructs with the following sequences were synthesized and sequence verified (Biomatik). The L. lactis strain IL1403 genes, gadB and gadC (genomic position 1326770-1323662, 3109 bp) were placed downstream of constitutive promoters, P2, P5 and shortened version of P8 (P8s) (Zhu et al. FEMS Microbiol Lett 2015, 362) (Table 2, FIGS. 1A, 1B) The ribosome binding sites associated with P2, P5 and P8s were fused upstream of the gadC start codon. The P2-gadCB, P5-gadCB and P8s-gadCB sequences were placed antisense and within the upstream, leuA fragment 1, (genomic position 1239855-1240223, 369 bp) and downstream, leuA fragment 2 (genomic position 1241340-1241054, 287 bp) regions of the L. lactis strain IL1403 leuA gene. Pstl restrictions sites incorporated at the ends of these constructs were used for cloning into the L. lactis plasmid, pGh9: ISS1 ((Maguin et al. J. Bacteriol. 1996, 178, 931-935). Recombinant plasmids, pGh9: ISS1-P2, pGh9: ISS1-P5 and pGh9: ISS1-P8s were transformed into L. lactis strain IL1403 and the resulting L. lactis strains were named, pGh-P2-GAD-L. lactis, pGh-P5-GAD-L. lactis and pGh-P8s-GAD-L. lactis. These strains were used for RTqPCR experiments to determine expression of gadB and for quantitating GABA production.

Bacterial transformation. Escherichia coli MC1061 electrocompetent cells (Lucigen) were transformed using standard electroporation conditions (BioRad Pulser) and recovered at the pGh9: ISS1 replication permissive temperature of 30° C. for 1 hr in super optimal broth (SOB) before culturing on LB agar+erm for plasmid selection. Lactococcus lactis electrocompetent cells (Intact Genomics) were transformed (BioRad Pulser) using the following settings, 2,500 V, 25 uFD, and 400 ohms, and recovered at 30° C. for 1.5 hrs in GM17 broth prior to culturing on GM17+erm for plasmid selection.

Integration of Px-GAD constructs into the L. lactis chromosome. Integration of the Px (2, 5, or 8s)-GAD constructs at the L. lactis leuA locus requires use of a plasmid that allows for locus specific integration. pGh9: ISS1 contains the transposon ISS1 that integrates randomly into the genome. The Pstl Px-GAD fragment (3.9 kb) from the pGh9: ISS1-Px plasmids can be cloned into the Pstl site of pBVGh ((Blancato and Magni, Appl. Microbiol. 2010, 50, 542-546) to generate pBVGh-Px-GAD recombinant plasmids. The plasmids can be used to transform L. lactis to erythromycin resistance. Integration can be accomplished by culturing the transformed L. lactis strains at 37° C. in GM17+erm. Excision of the pBVGh backbone can be stimulated by three cycles of dilution and growth at 30° C. and 37° C. under nonselective culture conditions (GM17) as described in Blancato and Magni, 2010 ((Blancato and Magni, Appl. Microbiol. 2010, 50, 542-546). Recombination events that remove pBVGh and leave Px-GAD constructs at the leuA locus can be identified by PCR analysis of the genomic DNA of erms, white L. lactis colonies using oligos, leuA upstream and leuA downstream (Table 3).

Reverse transcription-quantitative PCR (RTqPCR). Fresh overnight cultures of L lactis strains (L. lactis unmodified, pGh9: ISS1-L. lactis, pGh-P2-GAD-L. lactis, pGh-P5-GAD-L. lactis and pGh-P8s-GAD-L. lactis) were diluted to OD600 0.2 and incubated until they reached mid-log phase, OD600 1.5. Total RNA was isolated from L. lactis pelleted from 5 mL culture using a Fungal/Bacterial RNA miniprep kit (Zymo Research). Reverse transcription reactions were carried out on 100 ng RNA using the ReverTra Ace™ qPCR RT Master Mix with gDNA Remover (Toyobo). The qPCR reactions were carried out on 1 μl of a 1:10 dilution of the RT reaction with THUNDERBIRD™ Next SYBR™ qPCR Mix (Toyobo). Relative expression of gadB (gadB-RT3F and gadB-RT3R, Table 3) was determined using 16s rRNA (16s RT2F and 16s RT2R, Table 3) as a reference gene. The ΔΔCt was calculated by comparing the ΔCt of strains with plasmids to the ΔCt of the L. lactis IL1403 strain without a plasmid. The fold-change in expression was calculated as 2-44Ct. A Kruskal-Wallis (ANOVA) test was used determine the difference among means and a Dunnett's multiple comparison test was used to compare means between groups.

GABA ELISA. A GABA ELISA (LDN®, GABA ELISA) was used to analyze supernatants collected from L. lactis strains (pGh9: ISS-L. lactis and pGh-P8-GAD-L. lactis). Strains were cultured in GM17+erm overnight and then diluted to OD600 0.2 in GM17+erm alone or GM17 with glutamic acid HCl (50 mM, 150 mM or 200 mM) and incubated at 30° C. for the indicated times. Colony forming units per mL (CFU/mL) at the time of sample collection was determined using a spectrophotometer (\600 nm). One mL samples were collected, centrifuged (3 minutes, 10,000 rpms) to remove cells, and stored at −80° C. until analyzed for GABA. GABA concentrations of experimental samples and media only controls (GM17+erm or GM17+erm and glutamic acid, 50 mM, 150 mM or 200 mM) were determined using a standard curve generated with each ELISA. GABA concentration is reported as GABA concentration of the sample minus the GABA concentration of the media control, and normalized for CFU/mL.

Table 2. Promoters identified by Zhu et al. FEMS Microbiol Lett 2015, 362 were used or modified for use in this study. The exact Zhu et al. 2015 P2, P5 and P8 sequences are included below. A shortened version of P8 used in this study is indicated by lowercase text and the proposed −10 and −35 promoter and ribosome binding site consensus sequences are indicated in bold and underlined text, respectively.

| Promoter | sequence |
|---|---|
| P2 | TTTCTCCTATCTTTTCTATTTGACATCTAAATCCATTATAAGGCAAAGT |
| | GTATTAAAAAGACAGCTTCACTATGATTAGTACACAAATAATCATTTA |
| | AAAAGTGTAAATAATTTTTTATAAATAACGAATCAAAAAGTTTGACCA |
| | TTACTGACCAAAGTATTATAATTAGAATGTAGTGAGAAAAAGATAATA |
| | ATTACTACTAACTATGAAATCGAAATGAGGTGTTTTT (SEQ ID NO: |
| | 12) |
| P5 | GAAAAAGAAAATGTTTTTGTATTTTTAGAATCCCTTTTCTATAAATCAA |
| | TTCTAATTATAAGGACCTGATGATTGAGTGATAATGCTAGTTTGAAGC |
| | ATTCTTAGTAAGAAAGTGATTTTTTATAAATGGTTTATAGAATAAATT |
| | GTACAGCGTTTAATTGGACTTGCTCTCTGAAATAACGTAAAATTGTA |
| | GTGAGGAGGACGGTTACA (SEQ ID NO: 13) |
| P8 | GATAAAATTTCTAATGAtttttttaggacaattatttctcataaaaagcagattttagaaaga |
| | aaattgtatttttttaacagctttgactgccctttttggaagagtttatgtataatagaattagttagttttgctat |
| | tgatatagcagcagaaatggagagatatA (SEQ ID NO: 6) |
| P8s | tttttttaggacaattatttctcataaaaagcagattttagaaagaaaattgtatttttttaacagctttgact |
| | gccctttttggaagagtttatgtataatagaattagttagttttgctattgatatagcagcagaaatggag |
| | agatat (SEQ ID NO: 5) |

Biomatik promoter clone sequences (promoter sequence is bolded and tails are unbolded):

| | |
|---|---|
| P2 w/tails | AGATCTGCATCCAGTGAACCTGCCCCCTATTC |
| | TTTCTCCTATCTTTTCTATTTTGACATCTAAATCCATTATAAGGCAAAGTGT |
| | ATTAAAAAGACAGCTTCACTATGATTAGTACACAAATAATTCATTTAAAAA |
| | GTGTAAATAATTTTTTATAAATAACGAATCAAAAAGTTTGACCATTACTGA |
| | CCAAAGTATTATAATTAGAATGTAGTGAGAAAAAGATAATAATTACTACTA |
| | ACTATGAAATCGAAATGAGGTGTTTTT |
| | GATGAATCAAAAAAAAATATCATTATTCGGAGATCT (SEQ ID NO: 14) |
| P5 w/tails | AGATCTGCATCCAGTGAACCTGCCCCCTATTC |
| | GAAAAAGAAAATGTTTTTGTATTTTTAGAATCCCTTTTCTATAAATCAATTC |
| | TAATTATAAGGACCTGATGATTGAGTGATAATGCTAGTTTGAAGCATTCTT |
| | AGTAAGAAAGTGATTTTTTATAAATGGTTTATAGAATAAATTGTACAGCGT |
| | TTAATTGGACTTGCTCTCTGAAATAACGTAAAATTGTAGTGAGGAGGACG |
| | GTTACA |
| | GATGAATCAAAAAAAAATATCATTATTCGGAGATCT (SEQ ID NO: 15) |
| P8s | AGATCTGCATCCAGTGAACCTGCCCCCTATTC |
| w/tails | TTTTTTTAGGACAATTATTTCTCATAAAAAGCAGATTTTAGAAAGAAAATTG |
| | TATTTTTTTAACAGCTTTGACTGCCCTTTTTGGAAGAGTTTATGTATAATAG |
| | AATTAGTTAGTTTTGCTATTGATATAGCAGCAGAAATGGAGAGATAT |
| | GATGAATCAAAAAAAAATATCATTATTCGGAGATCT (SEQ ID NO: 16) |

Table 3. Oligonucleotides used for PCR and qPCR.

| Oligonucleotide | sequence |
|---|---|
| leuA-upstream | CGAGGACCGAGAGACGTCCTCACG (SEQ ID NO: 17) |
| leuA-downstream | GCCCAATTCCATCTCCCGCAAGTGTC (SEQ ID NO: 18) |
| RT3-gadB-F | ATGCTCCCTTTGTTGAGCCA (SEQ ID NO: 19) |
| RT3-gadB-R | ACGCCACAAAACCCAACCTA (SEQ ID NO: 20) |
| LI16srRNA2-F | ACGAGACTGCCGGTGATAAA (SEQ ID NO: 21) |
| LI16srRNA2-R | GAGTTGCAGCCTACAATCCG (SEQ ID NO: 22) |

Animals. Female C57BL/6 mice were obtained from Envigo (Envigo RMS, Inc., Indianapolis, IN, USA), except in one experiment where another provider was also used (The Jackson Laboratory, Inc., Bar Harbor, ME, USA). The mice arrived at Eastern Washington University when they were 8 weeks old and were given time to acclimate before immunization at 10 weeks. At the time of disease induction, they weighed 20 g. They were housed in Eastern Washington University's vivarium in wire-top cages (46 cm×25 cm×20 cm) with bedding. Animals were placed in cages randomly with 5 animals per cage. The room environment was kept at 22±1° C. and 23-33% humidity with a 12-hour light/dark cycle. All animals had free access to food and water. When mice reached an EAE clinical score of 2.5 or higher crushed food soaked in water was placed in a shallow dish at the bottom of the cage to help facilitate access. Mice were fed Teklad 2018 pellet food containing plenty of glutamate for bacterial synthesis of GABA. All animal care and procedures followed Eastern Washington University's Institutional Animal Care and Use Committee (IACUC) policies and approved protocols.

EAE Induction and Clinical Scoring. Mice were induced with EAE using the Hook Kit™ for EAE induction (Hooke Laboratories, EK-2110). To induce, each mouse was given a subcutaneous injection of 250 μg myelin oligodendrocyte glycoprotein 35-55 (MOG35-55) emulsified in complete Freund's adjuvant on the flanks at 2 sites on day 0. The same day and 48 hours later the mice received an intraperitoneal injection of 200 ng of Bordetella pertussis toxin (200 μL). In this model, the onset of the disease (where symptoms are first observed) is typically around days 9-14 and the peak of the disease occurs 3-4 days later.

All mice were monitored for the duration of the experiment, up to 28 days, and scored daily using the EAE clinical score scale for disease severity. The clinical scores are based on the degree of paralysis the animal exhibits. 0 is a healthy animal with no disease; 0.5, a distal limp tail; 1, completely limp tail or isolated weakness of gait without a limp tail; 1.5, a limp tail and hind limb weakness; 2, unilateral partial hind limb paralysis; 2.5, bilateral partial hind limb paralysis; 3, complete bilateral hind limb or partial hind and front limb paralysis; 3.5, complete bilateral hind limb paralysis and partial front limb paralysis. 5, moribund or dead animal. In accordance with IACUC policies to minimize undue suffering, mice displaying a score of 3.5 or higher (unable to right themselves when placed on their sides) for more than two consecutive days were euthanized. Thereafter the mouse was listed as a 5 (the clinical score for a dead mouse). Mice were euthanized via carbon dioxide asphyxiation followed by cervical dislocation. Body weights were measured weekly and expressed as % body weight at time of EAE induction.

Characterization of GABA produced by GAD-L. lactis. A preliminary assay was done with supernatants from each strain of L. lactis (P8s-GAD-L. lactis, pGh9: ISS1-L. lactis, WT-L. lactis, P2-GAD-L. lactis, and P5-GAD-L. lactis) at stationary growth phase, after samples were centrifuged to remove cells. The supernatants from the strains were tested in triplicate with a GABA-specific enzyme-linked immunosorbent assay (ELISA) using a GABA ELISA (LDN® BA E-2500), with assay controls provided by the ELISA kit, and pGh9: ISS1-L. lactis and WT L. lactis as controls for GABA production by P8s-GAD-L. lactis.

GABA levels produced by the different strains at different growth phases were compared. To create a growth curve, stationary phase bacteria were grown overnight in GM17 broth and 2 replicates of each strain were diluted to an OD600 0.2 (optical density) with GM17 media except for P2-GAD-L. lactis (which was removed due to low GABA production levels). After 1 hour of growth, their OD600 levels were tested and every ½ hour afterwards. Samples were plated to obtain colony forming unit counts (CFUs) and supernatants were collected at OD600 0.5, 1.0, 1.5 and 2 for ELISAs after centrifugation (to remove cells).

Collected samples along with GM17 media as a control were centrifuged and cell pellets removed then tested with a GABA ELISA kit (AVIVA Systems Biology, GABA ELISA Kit OKEH02564). The supernatant samples of P8s-GAD-L. lactis, pGh9: ISS1-L. lactis, P5-GAD-L. lactis, and the GM17 media were all in triplicate. WT-L. lactis was run in duplicate. The ELISA was run twice with both replicates from the growth curve experiment and results were combined.

Treatment with GAD L. lactis and EAE severity. Experiments were conducted to determine the impact of P8s-GAD-L. lactis on EAE severity. Mice were randomly divided into 3 treatment groups: a medium only group (medium), a pGh9: ISS1-L. lactis group (pGh9: ISS1), and a P8s-GAD-L. lactis group (P8s). The medium group was treated with 0.1 mL of GM17 media. The pGh9: ISS1 and P8s groups were treated with 5×10⁸ CFU a day of their respective strains of bacteria suspended in 0.1 mL of GM17 media. Mice were treated from day 0 to end of the experiment, 5 times a week (Robert et al. Diabetes 2014, 63, 2876-2887). Treatments were administered via oral gavage. Mice were weighed weekly and EAE clinical scores were measured daily.

The P8s and pGh9: ISS1 bacterial cultures for treatment were started weekly on GM17 agar plates containing 5 μg/mL erythromycin from −80° C. archived strains. Incubated at 30° C., these plates were then used to inoculate overnight cultures of ERM GM17 media. New ERM GM17 media was made every 3 days and it was stored at 3° C. Morning cultures were diluted to OD600 0.2. The bacteria were incubated for 3 hours to OD600 1.5 at 30° C. The ERM GM17 media was removed by centrifugation and the cell pellets were resuspended in GM17 media to achieve 5×10⁸ CFU per 0.1 mL media. New overnight cultures were made daily with a dilution of 2 mL broth to 25 μL previous overnight culture.

Statistics. Repeated measures and mixed-effect ANOVA followed by Tukey's multiple comparison post-hoc test was used to estimate group differences for EAE clinical scores, body weights, and body weight changes. Group differences in disease onset and disease severity were evaluated using non-parametric Kruskal-Wallis followed by Dunn's multiple comparisons tests. Means of the GABA levels quantified by ELISA were compared by mixed-effect ANOVA followed by Tukey's multiple comparison post-hoc test. P values below 0.05 were considered significant.

### Example 7

This Example demonstrated that treatment of mice with P8s-GAD-L. lactis, a genetically engineered L. lactis with enhanced GABA production, is protective against colitis induced in the mice as measured by survival and colon length.

Inflammatory bowel disease (IBD) is a chronic disease of the gastrointestinal (GI) tract characterized by an inflammatory reaction that includes Crohn's disease and ulcerative colitis. Both conditions cause a plethora of symptoms, including diarrhea, rectal bleeding, pain, and abdominal cramps. In ulcerative colitis, the inflammatory process and lesions are associated with the colon and the rectum, while in Crohn's disease, the immunopathological signs of the disease can be observed throughout the entire GI tract. Multiple animal models exist for the study of IBD.

The 2,4,6-trinitrobenzenesulfonic acid (TNBS) model of IBD in BALB/c mice was used, whereby colitis was induced by the administration of TNBS via rectum. The colitis results in body weight loss and high mortality. The disease promotes intestinal epithelium disruption, inflammation, and shortness of colon length. To test the immunomodulatory effects of GAD L. lactis probiotic bacterium, the TNBS model was used to compare survival rates and colon length in mice treated orally with P8s-GAD-L. lactis, L. lactis (P), and sterile water.

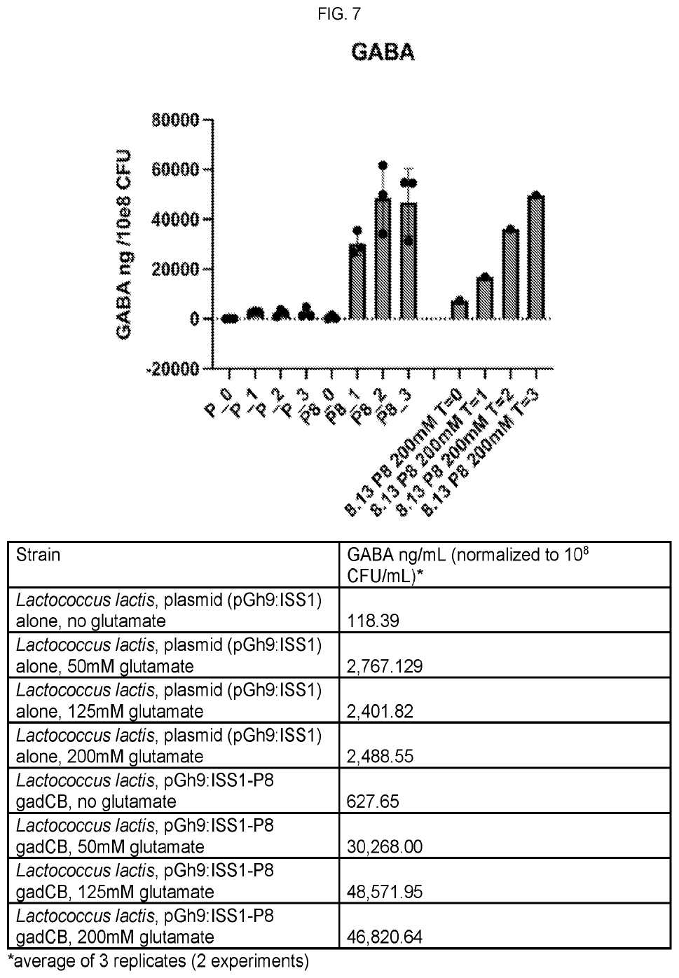

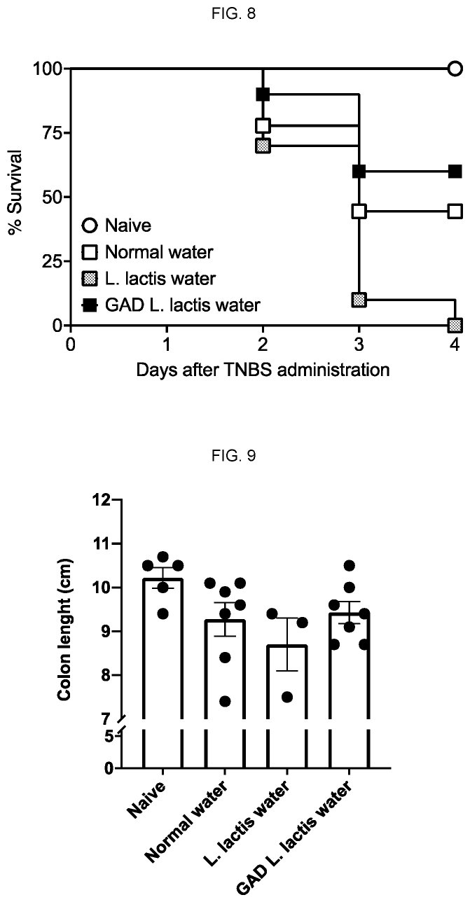

Ten (10) female BALB/c mice at 8 weeks old were randomly divided into four groups: Group 1, Colitis group control (n=10), drinking normal water for two weeks before and after colitis induction; Group 2, The L. lactis (P) group (n=10), drinking L. lactis (pGh9: ISS1 plasmid only) drinking water for two weeks before and after the colitis induction. Group 3, The P8s-GAD-L. lactis group (n=10) received P8s-GAD-L. lactis in their drinking water for two weeks before and after colitis induction. Group 4, Healthy control group (n=5). The concentration of L. lactis strains in the drinking water was 5×10⁸ CFU/mouse. The drinking water used for all treatments was deionized and autoclaved. Disease was induced in two stages: on day-7, mice of groups 1, 2, and 3 were pre-sensitized so the skin on the back of the mouse was shaved for a 1.5×1.5 cm square. Sensitization causes mice to be more susceptible to disease. 150 μl of TNBS solution as a 5% TNBS emulsion (4 volumes of acetone/olive oil), was applied to the shaved skin. On day 0, the TNBS solution was administered into the rectum. A 3.5 F catheter connected to a 1 ml syringe was used to administer 100 μl of 5% TNBS solution (weight/volume) in autoclaved water and 1 volume of absolute ethanol. Disease was monitored for five days, and survival rates and colon length determined. The P8s-GAD-L. lactis group had a 60% survival rate, greater than the 0% survival rate of group 2 (L. lactis (P)) (FIG. 8). The length of the colon was on average 0.73 cm longer in the P8s-GAD-L. lactis group than in the L. lactis (P) group (FIG. 9). The P8-GAD-L. lactis had a 92% colon length retention (compared to the naive mice (group 4)), and higher than mice in the L. lactis (P) group (85% colon length retention) in comparison to the naive group (FIG. 9).

### REFERENCES

CN110257447A
KR20060057398A
Bhat, R.; Axtell, R.; Mitra, A.; Miranda, M.; Lock, C.; Tsien, R. W.; Steinman, L. Inhibitory Role for GABA in Autoimmune Inflammation. Proceedings of the National Academy of Sciences of the United States of America 2010, 107, 2580-2585.
Blancato, V. S.; Magni, C. A Chimeric Vector for Efficient Chromosomal Modification in Enterococcus Faecalis and Other Lactic Acid Bacteria. Lett. Appl. Microbiol. 2010, 50, 542-546, doi: 10.1111/j. 1472-765X.2010.02815.x.
Bolotin, A.; Wincker, P.; Mauger, S.; Jaillon, O.; Malarme, K.; Weissenbach, J.; et al. The Complete Genome Sequence of the Lactic Acid Bacterium Lactococcus lactis ssp. lactis IL1403. Genome Res. 2001, 11, 731-753.
Bravo, J. A.; Forsythe, P.; Chew, M. V.; Escaravage, E.; Savignac, H. M.; Dinan, T. G.; Bienenstock, J.; Cryan, J. F. Ingestion of Lactobacillus strain regulates emotional behavior and central GABA receptor expression in a mouse via the vagus nerve. Proceedings of the National Academy of Sciences, 2011, 108 (38), 16050-16055.
Cao, G.; Edden, R. A. E.; Gao, F.; Li, H.; Gong, T.; Chen, W.; Liu, X.; Wang, G.; Zhao, B. Reduced GABA Levels Correlate with Cognitive Impairment in Patients with Relapsing-Remitting Multiple Sclerosis. European radiology 2018, 28, 1140-1148.
Colombo, M.; Castilho, N. P. A.; Todorov, S. D.; Nero, L. A. Beneficial Properties of Lactic Acid Bacteria Naturally Present in Dairy Production. BMC Microbiol 2018, 18, 219, doi: 10.1186/s12866-018-1356-8.
De Stefano, N.; Giorgio, A. GABA: A New Imaging Biomarker of Neurodegeneration in Multiple Sclerosis? Brain 2015, 138, 2467-2468.
Enoch, M-A. The Role of GABA_{A} Receptors in the Development of Alcoholism. Pharmacol Biochem Behav. 2008, 90 (1), 95-104.
Guo, T.; Kong, J.; Zhang, L.; Zhang, C.; and Hu, S. Fine Tuning of the Lactate and Diacetyl Production through Promoter Engineering in Lactococcus lactis. PLOS ONE 2012, 7, e36296. doi: 10.1371/journal.pone.0036296.
Hyland, N. P., & Cryan, J. F. A gut feeling about GABA: focus on GABA_{B} receptors. Frontiers in pharmacology, 2010, 1, 124.
Jensen, P. R.; and Hammer, K. Artificial promoters for metabolic optimization. Biotechnol. Bioeng. 1998, 58, 191-195.
Jensen, P. R.; and Hammer, K. The Sequence of Spacers between the Consensus Sequences Modulates the Strength of Prokaryotic Promoters. Appl. Environ. Microbiol. 1998, 64, 82-87. doi: 10.1128/AEM.64.1.82-87.1998.
Lindholm, A.; and Palva, A. Constitutive production of the receptor-binding domain of the F18 fimbriae on the surface of Lactococcus lactis using synthetic promoters. Biotechnol Lett 2009, 32, 131. doi: 10.1007/s10529-009-0122-2.
Lyu, C.; Zhao, W.; Peng, C.; Hu, S.; Fang, H.; Hua, Y.; Yao, S.; Huang, J.; Mei, L. Exploring the contributions of two glutamate decarboxylase isozymes in Lactobacillus brevis to acid resistance and γ-aminobutyric acid production. Microbial cell factories, 2018, 17 (1), 1-14.
Lyu, C. J.; Fei, J. Y.; Yan, J. P.; Xu, Q. C.; Mei, J. Q.; Yue, H. Y.; Chen, L.; Mei, L.; Huang, J.; Yao, S. J. Improvement of γ-aminobutyrate biosynthesis by genetically engineered Lactococcus lactis. Biochemical Engineering Journal, 2020, 157, 107525.
MacCormick C. A.; Griffin H. G.; Gasson M. J. Construction of a food-grade host/vector system for Lactococcus lactis based on the lactose operon. FEMS Microbiol Lett, 1995, 127, 105-109.
Maguin, E.; Prevost, H.; Ehrlich, S. D.; and Gruss, A. Efficient insertional mutagenesis in lactococci and other gram-positive bacteria. J. Bacteriol. 1996, 178, 931-935.
Mazzoli, R.; Pessione, E. The Neuro-Endocrinological Role of Microbial Glutamate and GABA Signaling. Frontiers in Microbiology 2016, 7, 1934.
Ngo, D-H.; and Vo, T. S. An Updated Review on Pharmaceutical Properties of Gamma-Aminobutyric Acid. Molecules. 2019, 24 (15), 2678.
Ogaugwu, C. E.; Cheng, Q.; Fieck, A.; Hurwitz, I.; and Durvasula, R. Characterization of a Lactococcus lactis promoter for heterologous protein production. Biotechnol Rep (Amst) 2017, 17, 86-92. doi: 10.1016/j.btre.2017.11.010.
Robert, S.; Gysemans, C.; Takiishi, T.; Korf, H.; Spagnuolo, I.; Sebastiani, G.; Van Huynegem, K.; Steidler, L.; Caluwaerts, S.; Demetter, P.; et al. Oral Delivery of Glutamic Acid Decarboxylase (GAD)-65 and IL10 by Lactococcus Lactis Reverses Diabetes in Recent-Onset NOD Mice. Diabetes 2014, 63, 2876-2887.
Song, A. A.-L.; In, L. L. A.; Lim, S. H. E.; Rahim, R. A. A Review on Lactococcus Lactis: From Food to Factory. Microbial cell factories 2017, 16, 55.
Stromnes, I. M.; Goverman, J. M. Active Induction of Experimental Allergic Encephalomyelitis. Nature Protocols 2006, 1, 1810-1819.
Takala T. M.; Saris P. E. A food-grade cloning vector for lactic acid bacteria based on the nisin immunity gene nisl. Appl Microbiol Biotechnol 2002, 59, 467-471.
Thibessard, A.; Fernandez, A.; Gintz, B.; Decaris, B.; Leblond-Bourget, N. Transposition of pGh9: ISS1 is random and efficient in Streptococcus thermophilus CNRZ368. Can. J. Microbiol. 2002, 48:473-478.
Tian, J.; Chau, C.; Hales, T. G.; Kaufman, D. L. GABA (A) Receptors Mediate Inhibition of T Cell Responses. Journal of Neuroimmunology 1999, 96, 21-28.
Tian, J.; Lu, Y.; Zhang, H.; Chau, C. H.; Dang, H. N.; Kaufman, D. L. Gamma-Aminobutyric Acid Inhibits T Cell Autoimmunity and the Development of Inflammatory Responses in a Mouse Type 1 Diabetes Model. The Journal of Immunology 2004, 173, 5298-5304.
Tian, J.; Yong, J.; Dang, H.; Kaufman, D. L. Oral GABA Treatment Downregulates Inflammatory Responses in a Mouse Model of Rheumatoid Arthritis. Autoimmunity 2011, 44, 465-470, doi: 10.3109/08916934.2011.571223.
Vossen, J. M. van der; Lelie, D. van der; and Venema, G. Isolation and characterization of Streptococcus cremoris Wg2-specific promoters. Appl. Environ. Microbiol. 1987, 53, 2452-2457.
Yalçinkaya, N.; Akcan, U.; Örçen, A.; Küçükali, C. \. I.; Türkoğlu, R.; Kürtüncü, M.; Tüzün, E. Reduced Fecal GABA Levels in Multiple Sclerosis Patients. Multiple Sclerosis and Related Disorders 2016, 9, 60-61.
Zhu, D.; Liu, F.; Xu, H.; Bai, Y.; Zhang, X.; Saris, P. E. J.; Qiao, M. Isolation of Strong Constitutive Promoters from Lactococcus Lactis Subsp. Lactis N8. FEMS Microbiol Lett 2015, 362, doi: 10.1093/femsle/fnv107.

1. A genetic construct comprising:
a constitutive promoter operably linked to:
a gene encoding a glutamic acid decarboxylase; and
a gene encoding a glutamate/GABA antiporter.

2. The genetic construct of claim 1, wherein the constitutive promoter comprises a P2, a P5, or a P8 promoter.

3. The genetic construct of claim 2, wherein the P2 promoter has a sequence as set forth in SEQ ID NO: 12; the P5 promoter has a sequence as set forth in SEQ ID NO: 13; or the P8 promoter has a sequence as set forth in SEQ ID NO: 5 or SEQ ID NO: 6.

4. The genetic construct of claim 1, wherein the gene encoding the glutamic acid decarboxylase comprises gadB.

5. The genetic construct of claim 4, wherein the gadB comprises Lactococcus lactis gadB having at least 80% sequence identity to a sequence as set forth in SEQ ID NO: 1.

6. The genetic construct of claim 1, wherein the gene encoding the glutamate/GABA antiporter comprises gadC.

7. The genetic construct of claim 6, wherein the gadC comprises Lactococcus lactis gadC having at least 80% sequence identity to a sequence as set forth in SEQ ID NO: 2.

8. The genetic construct of claim 1, wherein the genetic construct further comprises a selectable marker.

9. The genetic construct of claim 8, wherein the selectable marker confers erythromycin resistance.

10. The genetic construct of claim 1, wherein the promoter and the genes are part of an expression cassette.

11. The genetic construct of claim 10, wherein the genetic construct further comprises an upstream homology arm and a downstream homology arm flanking the expression cassette that are homologous to a gene in a probiotic bacterium.

12. The genetic construct of claim 11, wherein the upstream homology arm has a sequence having at least 90% sequence identity to the sequence as set forth in SEQ ID NO: 9.

13. The genetic construct of claim 11, wherein the downstream homology arm has a sequence having at least 90% sequence identity to the sequence as set forth in SEQ ID NO: 10.

14. The genetic construct of claim 1, wherein the genetic construct comprises a sequence as set forth in SEQ ID NO: 11.

15. The genetic construct of claim 1, wherein the genetic construct comprises 5′ to 3′: the promoter, the gene encoding a glutamate/GABA antiporter, and the gene encoding a glutamic acid decarboxylase.

16. A cell comprising a genetic construct of any one of claims 1-15.

17. The cell of claim 16, wherein the cell comprises a probiotic bacterium.

18. The cell of claim 17, wherein the probiotic bacterium comprises Lactococcus lactis.

19. A method of producing gamma-aminobutyric acid (GABA), comprising culturing the probiotic bacterium of claim 17 in media.

20. A method of preparing genetically modified probiotic bacteria that produce gamma-aminobutyric acid (GABA), comprising introducing a genetic construct of any one of claims 1-15 into probiotic bacteria to obtain genetically modified probiotic bacteria; and culturing the genetically modified probiotic bacteria in media.

21. The method of claim 20, wherein the culturing comprises growing the genetically modified probiotic bacteria at 20° C. to 50° C.

22. The method of claim 20, wherein the culturing further comprises adding glutamic acid HCl to the media.

23. The method of claim 20, wherein the culturing comprises growing the genetically modified probiotic bacteria to an OD600 of 0.5 to 2.

24. The method of claim 20, wherein the genetically modified probiotic bacteria produces an amount of GABA that is 2-fold to 200-fold greater as compared to an amount of GABA produced by a control.

25. The method of claim 24, wherein the control is probiotic bacteria of the same genus or species that have not been genetically modified or that have been genetically modified with a control plasmid.

26. The method of claim 20, wherein the genetically modified probiotic bacteria produce 500 ng/ml GABA to 60,000 ng/ml GABA.

27. A composition comprising the probiotic bacteria of claim 17 and a pharmaceutically acceptable carrier.

28. The composition of claim 27, wherein the composition comprises an oral composition.

29. The composition of claim 27, wherein the composition includes a solid or a liquid.

30. The composition of claim 29, wherein the solid includes a lyophilized powder.

31. The composition of claim 27, wherein the composition is part of a dairy product.

32. The composition of claim 31, wherein the dairy product comprises yogurt, milk, cheese, kefir, ice cream, or butter.

33. A method of treating a disease or disorder in a subject in need thereof comprising administering a therapeutically effective amount of the composition of claim 27.

34. The method of claim 33, wherein the disease or disorder is associated with GABA deficiency, impaired GABA receptor mediated signaling, and/or an excess of excitatory neurotransmitters.

35. The method of claim 34, wherein the disease or disorder associated with GABA deficiency, impaired GABA receptor mediated signaling, and/or an excess of excitatory neurotransmitters comprises alcoholism, depression, anxiety, autism, multiple sclerosis, schizophrenia, Parkinson's Disease, Huntington's disease, epilepsy, post-traumatic stress disorder (PTSD), or stroke and its complications.

36. The method of claim 33, wherein the disease or disorder is associated with inflammation.

37. The method of claim 33, wherein the disease or disorder is an inflammatory autoimmune disease.

38. The method of claim 37, wherein the inflammatory autoimmune disease comprises cachexia, inflammatory bowel diseases (IBD), psoriatic arthritis, rheumatoid arthritis, Type 1 diabetes, Type 2 Diabetes, Sjögren's syndrome, systemic lupus erythematosus, celiac disease, Graves' disease, Hashimoto's thyroiditis, Addison's disease, dermatomyositis, psoriasis, chronic inflammatory demyelinating polyneuropathy, Guillain-Barre syndrome, myasthenia gravis, or vasculitis.

39. The method of claim 38, wherein the IBD is Crohn's disease or ulcerative colitis.


SEQUENCE LISTING (27 sequences)

SEQ ID NO: 1 - protein, 466 aa, Lactococcus lactis
MLYGKENRDEAEFLEPIFGSESEQVDLPKYKLAQQSIEPRVAYQLVQDEMLDEGNARLNLATFCQTYMEPEAVKLMSQTLEKNAIDKSEYPRTTEIENRCVNMIADLWNASEKEKFMGTSTIGSSEACMLGGMAMKFSWRKRAEKLGLDINAKKPNLVISSGYQVCWEKFCIYWDIEMREVPMDKEHMSINLDKVMDYVDEYTIGVVGIMGITYTGRYDDIKALDNLIEEYNKQTDYKVYIHVDAASGGLYAPFVEPELEWDFRLKNVISINTSGHKYGLVYPGVGWVLWRDKKYLPEELIFKVSYLGGELPTMAINFSHSASQLIGQYYNFVRYGFDGYKAIHERTHKVAMFLAKEIEKTGMFEIMNDGSQLPIVCYKLKEDSNRGWNLYDLADRLLMKGWQVPAYPLPKNLENEIIQRLVIRADFGMNMAFNYVQDMQEAIEALNKAHILYHEEPENKTYGFTH

SEQ ID NO: 2 - protein, 503 aa, Lactococcus lactis
MNQKKISLFGFFALTASMVLTVYEYPTFATSKLHLVFFLLLGGLLWFLPVALCAAEMATVEGWKNGGIFSWVSQTLGERFGFAAIFFQWFQITVGFVTMIYFILGALSYVLNFQALNTDPLIKFIGLLIIFWGLTFSQLGGTQRTAKLVKAGFVVGIVIPSIILFGLAAAYFIGGNPIEIPINKHAFVPDFSQVSTLVVFVSFILAYMGVEASASHINELENPKRNYPLAMILLVILAISLDAIGGFSVAAVIPQKDLSLSAGVIQTFQTLILHFNHHLGWLVKVIALMIAFGVMGEVSSWVVGPSRGMFAAAQRGLLPKFLRKTNTHEVPVPLVMIQGIIVTLWGAVLTFGGGGNNLSFLVAISLTVVIYLVGYLLFFIGYFVLIYKKQNLKRTYNVPGKRVGKTIIAGIGFLLSIFALFISFVPPASIAKNETHTYQMILLISFVVTAILPFIVYELHDKRGHDTIEEPRHFKARDVNPAIYPAARGEHHIIKKEEHILKH

SEQ ID NO: 3 - DNA, 1401 bp, Lactococcus lactis
atgttatacggaaaagaaaatcgcgatgaagcagagttcttggaaccaatttttggttcagaaagtgaacaagtggatttgcctaaatataaattagctcaacaatcaattgaacctcgagtggcctatcagttagttcaagatgaaatgttagatgaagggaacgctcgtttaaatttagccacattctgtcaaacttatatggaacctgaagcagtcaaactaatgagtcaaaccttggaaaaaaatgcaattgataaatcggaatatccaagaacaactgaaattgagaaccgttgcgtcaacatgatcgctgacctttggaatgcgagtgaaaaagaaaaatttatggggacttcaacgattggttcttcagaagcttgtatgcttggtggaatggccatgaagttttcttggcgcaagcgagcagaaaaattgggattagatattaatgcgaaaaagccaaacttagttatttcatctggttatcaagtttgctgggaaaaattctgtatttattgggatattgaaatgcgagaagtgccaatggataaagaacatatgtcaatcaatttggacaaggtgatggattatgttgatgagtacacgattggtgtagttggtattatggggattacttatactggtcgttatgatgatatcaaagctttggataatttaattgaagaatataataaacagacagactataaagtttatattcacgtagatgctgcttcaggaggactttatgctccctttgttgagccagaacttgagtgggatttccgtttgaaaaatgtcatttcaatcaatacctcaggccataaatatggtttagtttatcctggtgtaggttgggttttgtggcgtgacaaaaaatatttaccagaagaattaatttttaaagtaagttatcttggaggagaactaccaacgatggccattaatttttctcatagtgcctctcaattaattggtcaatattataattttgtacgttatggatttgatggatataaagctattcatgagagaacacataaagtagccatgtttttagcaaaagaaattgaaaaaactggaatgtttgaaattatgaacgatgggtcacaattgccaattgtctgctataaattaaaagaagattcaaatcgaggttggaatctttatgatttggcggaccgtttattaatgaagggatggcaagtgcctgcttatccacttcccaaaaatttggaaaatgaaatcattcaacgtttagtgattcgagcagattttgggatgaatatggcatttaactatgttcaagatatgcaagaagcaattgaggctttaaataaggctcatattctatatcatgaagagcctgaaaataaaacatatggatttactcactaa

SEQ ID NO: 4 - DNA, 1512 bp, Lactococcus lactis
atgaatcaaaaaaaaatatcattattcggtttttttgcattaaccgcttcaatggttttgactgtctatgagtatccaacttttgccacgtcaaaattacatttggtgttctttttacttctcggaggactactctggtttttgcccgtagcgctctgtgcagcagaaatggcgacggttgaaggttggaaaaatggtggaatttttagttgggtgagccaaactttaggggagcgctttggttttgcagccatattttttcagtggtttcaaattacagttggctttgtcactatgatctatttcattttaggggccctctcttatgtgttaaattttcaggcacttaatacagatccattgattaaatttattggtttactaatcattttttggggattgacattttctcagttaggtgggacacaaagaactgctaaattggtaaaagctggttttgtagttgggatagtgattccatcgattatcttgtttggattagctgcggcatattttatcggaggtaatcctatagagataccaattaataagcatgcttttgtaccagatttttcacaagtatcaactttagtagtttttgtttcttttattcttgcttatatgggggtagaagcttcagcttcgcacattaatgaacttgaaaatccaaagagaaattatcccttagcaatgattttattagtaatattagctatttctttagatgccataggtggattttctgtagcagcggttattcctcaaaaagacttatcattaagtgcaggggtaattcaaacttttcaaacgttaatcttacattttaatcatcatttgggatggttagttaaagtaattgcactaatgattgcctttggggttatgggagaagtgagttcatgggttgttggtccttctagagggatgtttgcagcagcacaaagaggcttattaccaaaatttttacgtaaaacaaatacgcatgaagtccctgttcctttagttatgattcaaggaatcattgttacactttggggcgctgtattaacttttggaggaggaggaaataatttatctttcttagttgccatttcactgacagtagtgatttatttggtaggttatctcttattctttattggttactttgttttaatctataaaaaacaaaatttaaagcgcacttataatgttccaggtaaaagagttggaaaaacaatcattgcgggaattggatttttattatcaatttttgctctatttatttcctttgttcctccagcatcaattgcgaaaaatgagactcatacttaccaaatgatacttcttataagttttgttgtgactgctatcttgccatttattgtttatgaattgcatgataaaaggggacatgatactattgaagaaccaaggcactttaaagcaagagatgtgaaccccgcgatttatccagcagctcgtggtgagcatcatattattaaaaaagaagaacatatcttaaaacattaa

SEQ ID NO: 5 - DNA, 151 bp, Artificial Sequence
  synthesized P8 promoter (short)
tttttttaggacaattatttctcataaaaagcagattttagaaagaaaattgtatttttttaacagctttgactgccctttttggaagagtttatgtataatagaattagttagttttgctattgatatagcagcagaaatggagagatat

SEQ ID NO: 6 - DNA, 169 bp, Artificial Sequence
  synthesized P8 promoter
gataaaatttctaatgatttttttaggacaattatttctcataaaaagcagattttagaaagaaaattgtatttttttaacagctttgactgccctttttggaagagtttatgtataatagaattagttagttttgctattgatatagcagcagaaatggagagatata

SEQ ID NO: 7 - protein, 513 aa, Lactococcus lactis
MRKIEFFDTSLRDGEQTPGVSFSISEKVTIAKQLEKWRISVIEAGFSAASPDSFEAVKQIADSLNDTAVTALARCVISDIDKAVEAVKGAKYPQIHVFIATSPIHMKYKLKISPEEVLKNIDKCVRYARERVEVVEFSPEDATRTELNFLLEAVQTAVDAGATYINIPDTVGYTTPEEYGKIFKFLIDNTKSDREIIFSPHCHDDLGMAVANSLAAIKAGAGRVEGTVNGIGERAGNAALEEIAVALHIRKDFYQAQSPLKLSETAATAELISQFSGIAIPKNKAIVGANAFAHESGIHQDGVLKNAETYEIITPELVGIKHNSLPLGKLSGRHAFSEKLTELNIAYDDESLAILFEKFKKLADKKKEITDADIHALFTGETVKNLAGFILDNVQIDGHKALVQLKNQEEEIYVSQGEGSGSVDAIFKAIDKVFNHQLKLISYSVDAVTDGIDAQATTLVSVENLSTGTIFNAKGVDYDVLKGSAIAYMNANVLVQKENLQGKVEQISAHDGI

SEQ ID NO: 8 - DNA, 1542 bp, Lactococcus lactis
atgcgaaaaattgaattctttgacacaagtttgagagatggcgaacagacaccgggcgttagtttctccatttcagaaaaagtaacgattgctaaacaactggaaaaatggaggatttctgtcatagaggctggtttttctgcggcaagtccagatagttttgaagcagtaaagcaaattgctgattctttgaatgatacggctgtcactgcattagctcgctgtgttatttcagatatcgataaagcggttgaagcggtaaagggggctaaatatccgcaaattcatgttttcattgcaacttcacctattcacatgaaatataaacttaaaatcagtcccgaagaagttttgaaaaatattgataagtgtgtgagatacgcacgtgaacgggtcgaggttgttgagttttctccagaggatgcaacaagaacggagttgaattttcttttagaggctgttcaaacggctgtcgatgctggagcaacttatattaatattcctgacactgtcggttatacgacaccagaagaatatggaaaaatttttaaatttttgattgataatactaagtctgaccgagaaattatttttagtccacattgtcatgatgatttaggaatggctgtagctaattcattagctgcaattaaagctggggctgggagagttgaaggaactgtcaatggtattggagagcgagctgggaatgctgctcttgaagaaattgctgtggcactacatattcgtaaagatttttatcaggcacaaagtcctttaaaactttcagaaactgctgcaacggcagaactaatttcacaattttcaggaattgctattccaaaaaataaagcaattgttggtgctaatgcttttgcacacgaatcaggaattcatcaagatggtgtccttaaaaatgctgaaacttatgaaattattacaccagaacttgtcggaataaagcataattcgttgcctttaggtaaactttctggtcgtcatgcttttagtgaaaaattgacggaacttaatattgcttatgacgatgaaagtcttgcaattttatttgaaaaatttaaaaaattagctgacaagaaaaaagaaattactgacgcagatattcatgccttgtttacaggagaaacggtaaaaaatctagctggatttatacttgataatgttcaaattgatgggcacaaggcattggtgcaactaaaaaatcaagaagaggaaatttatgttagccaaggagaggggtcaggttcagtggatgcaatttttaaagctattgataaagtctttaatcatcaactaaaattaatttcctattcagttgatgctgtaactgatggaattgatgcacaagcaacgactttggtttctgttgaaaatctatctacaggcactatatttaatgctaaaggtgttgattatgatgtattgaaaggaagcgccattgcttacatgaacgctaatgttttagttcaaaaagaaaatttacaaggaaaggttgaacaaatttcagctcatgatggaatttaa

SEQ ID NO: 9 - DNA, 369 bp, Artificial Sequence
  synthesized upstream leuA homology arm
caagtttgagagatggcgaacagacgccgggcgttagtttctgcatttcagaaaaagtaacgattgctaaacaactggaaaaatgggggatttctgtcatagaggctggttttcctgcggcaagtccagatagttttgaagcagtaaagcaaattgctgattctttgaatgatacggctgtcactgcattagctcgttgtgttatctcagatatcgataaagcggttgaagcggtaaagggggctaaatatccgcaaattcatgttttcattgccacttcacctattcacatgaaatataaacttaaaatcagtcccgaagaagttttgaaaaatattgataaatgtgtgaaatacgcacgtgaacggg

SEQ ID NO: 10 - DNA, 262 bp, Artificial Sequence
  synthesized downstream leuA homology arm
gaatagggggcaggttcactggatgcaatttttaaagctattgataaagtctttaatcatcaactaaaattaatttcctattcagttgatgctgtaactgatggaattgatgcacaagcaacgactttggtttctgttgaaaatctatctacaggcactatatttaatgctaaaggtgttgattatgatgtattgaaaggaagcgccattgcttacatgaacgctaatgttttagttcaaaaagaaaatttacaaggaaagg

SEQ ID NO: 11 - DNA, 3933 bp, Artificial Sequence
  synthesized genetic construct including upstream leuA homology arm, P8 short promoter, gadC gene, gadB gene, and downstream leuA homology arm
ctgcagccatcatgagctgaaatttgttcaacctttccttgtaaattttctttttgaactaaaacattagcgttcatgtaagcaatggcgcttcctttcaatacatcataatcaacacctttagcattaaatatagtgcctgtagatagattttcaacagaaaccaaagtcgttgcttgtgcatcaattccatcagttacagcatcaactgaataggaaattaattttagttgatgattaaagactttatcaatagctttaaaaattgcatccagtgaacctgccccctattctttttttaggacaattatttctcataaaaagcagattttagaaagaaaattgtatttttttaacagctttgactgccctttttggaagagtttatgtataatagaattagttagttttgctattgatatagcagcagaaatggagagatatgatgaatcaaaaaaaaatatcattattcggtttttttgcattaaccgcttcaatggttttgactgtctatgagtatccaacttttgccacgtcaaaattacatttggtgttctttttacttctcggaggactactctggtttttgcccgtagcgctctgtgcagcagaaatggcgacggttgaaggttggaaaaatggtggaatttttagttgggtgagccaaactttaggggagcgctttggttttgcagccatattttttcagtggtttcaaattacagttggctttgtcactatgatctatttcattttaggggccctctcttatgtgttaaattttcaggcacttaatacagatccattgattaaatttattggtttactaatcattttttggggattgacattttctcagttaggtgggacacaaagaactgctaaattggtaaaagctggttttgtagttgggatagtgattccatcgattatcttgtttggattagctgcggcatattttatcggaggtaatcctatagagataccaattaataagcatgcttttgtaccagatttttcacaagtatcaactttagtagtttttgtttcttttattcttgcttatatgggggtagaagcttcagcttcgcacattaatgaacttgaaaatccaaagagaaattatcccttagcaatgattttattagtaatattagctatttctttagatgccataggtggattttctgtagcagcggttattcctcaaaaagacttatcattaagtgcaggggtaattcaaacttttcaaacgttaatcttacattttaatcatcatttgggatggttagttaaagtaattgcactaatgattgcctttggggttatgggagaagtgagttcatgggttgttggtccttctagagggatgtttgcagcagcacaaagaggcttattaccaaaatttttacgtaaaacaaatacgcatgaagtccctgttcctttagttatgattcaaggaatcattgttacactttggggcgctgtattaacttttggaggaggaggaaataatttatctttcttagttgccatttcactgacagtagtgatttatttggtaggttatctcttattctttattggttactttgttttaatctataaaaaacaaaatttaaagcgcacttataatgttccaggtaaaagagttggaaaaacaatcattgcgggaattggatttttattatcaatttttgctctatttatttcctttgttcctccagcatcaattgcgaaaaatgagactcatacttaccaaatgatacttcttataagttttgttgtgactgctatcttgccatttattgtttatgaattgcatgataaaaggggacatgatactattgaagaaccaaggcactttaaagcaagagatgtgaaccccgcgatttatccagcagctcgtggtgagcatcatattattaaaaaagaagaacatatcttaaaacattaaaaaattggaggatgtacctatgttatacggaaaagaaaatcgcgatgaagcagagttcttggaaccaatttttggttcagaaagtgaacaagtggatttgcctaaatataaattagctcaacaatcaattgaacctcgagtggcctatcagttagttcaagatgaaatgttagatgaagggaacgctcgtttaaatttagccacattctgtcaaacttatatggaacctgaagcagtcaaactaatgagtcaaaccttggaaaaaaatgcaattgataaatcggaatatccaagaacaactgaaattgagaaccgttgcgtcaacatgatcgctgacctttggaatgcgagtgaaaaagaaaaatttatggggacttcaacgattggttcttcagaagcttgtatgcttggtggaatggccatgaagttttcttggcgcaagcgagcagaaaaattgggattagatattaatgcgaaaaagccaaacttagttatttcatctggttatcaagtttgctgggaaaaattctgtatttattgggatattgaaatgcgagaagtgccaatggataaagaacatatgtcaatcaatttggacaaggtgatggattatgttgatgagtacacgattggtgtagttggtattatggggattacttatactggtcgttatgatgatatcaaagctttggataatttaattgaagaatataataaacagacagactataaagtttatattcacgtagatgctgcttcaggaggactttatgctccctttgttgagccagaacttgagtgggatttccgtttgaaaaatgtcatttcaatcaatacctcaggccataaatatggtttagtttatcctggtgtaggttgggttttgtggcgtgacaaaaaatatttaccagaagaattaatttttaaagtaagttatcttggaggagaactaccaacgatggccattaatttttctcatagtgcctctcaattaattggtcaatattataattttgtacgttatggatttgatggatataaagctattcatgagagaacacataaagtagccatgtttttagcaaaagaaattgaaaaaactggaatgtttgaaattatgaacgatgggtcacaattgccaattgtctgctataaattaaaagaagattcaaatcgaggttggaatctttatgatttggcggaccgtttattaatgaagggatggcaagtgcctgcttatccacttcccaaaaatttggaaaatgaaatcattcaacgtttagtgattcgagcagattttgggatgaatatggcatttaactatgttcaagatatgcaagaagcaattgaggctttaaataaggctcatattctatatcatgaagagcctgaaaataaaacatatggatttactcactaaaaaaaccgatattcatcttaagaaaaatcaaaagcgatatagttaatctatatcgcttttattttgatgataaattttgtttttttctatttagtcataagtttgtcagtgattatttttcataatatttatctaaaatctccatttttaaaggagtaatcttagataatggggctgcccgttcacgtgcgtatttcacacatttatcaatatttttcaaaacttcttcgggactgattttaagtttatatttcatgtgaataggtgaagtggcaatgaaaacatgaatttgcggatatttagccccctttaccgcttcaaccgctttatcgatatctgagataacacaacgagctaatgcagtgacagccgtatcattcaaagaatcagcaatttgctttactgcttcaaaactatctggacttgccgcaggaaaaccagcctctatgacagaaatcccccatttttccagttgtttagcaatcgttactttttctgaaatgcagaaactaacgcccggcgtctgttcgccatctctcaaacttgtgtcctgcag

SEQ ID NO: 12 - DNA, 230 bp, Artificial Sequence
  synthesized P2 promoter
tttctcctatcttttctatttgacatctaaatccattataaggcaaagtgtattaaaaagacagcttcactatgattagtacacaaataatcatttaaaaagtgtaaataattttttataaataacgaatcaaaaagtttgaccattactgaccaaagtattataattagaatgtagtgagaaaaagataataattactactaactatgaaatcgaaatgaggtgttttt

SEQ ID NO: 13 - DNA, 210 bp, Artificial Sequence
  synthesized P5 promoter
gaaaaagaaaatgtttttgtatttttagaatcccttttctataaatcaattctaattataaggacctgatgattgagtgataatgctagtttgaagcattcttagtaagaaagtgattttttataaatggtttatagaataaattgtacagcgtttaattggacttgctctctgaaataacgtaaaattgtagtgaggaggacggttaca

SEQ ID NO: 14 - DNA, 300 bp, Artificial Sequence
  P2 with tails promoter clone sequence
agatctgcatccagtgaacctgccccctattctttctcctatcttttctattttgacatctaaatccattataaggcaaagtgtattaaaaagacagcttcactatgattagtacacaaataattcatttaaaaagtgtaaataattttttataaataacgaatcaaaaagtttgaccattactgaccaaagtattataattagaatgtagtgagaaaaagataataattactactaactatgaaatcgaaatgaggtgtttttgatgaatcaaaaaaaaatatcattattcggagatct

SEQ ID NO: 15 - DNA, 278 bp, Artificial Sequence
  P5 with tails promoter clone sequence
agatctgcatccagtgaacctgccccctattcgaaaaagaaaatgtttttgtatttttagaatcccttttctataaatcaattctaattataaggacctgatgattgagtgataatgctagtttgaagcattcttagtaagaaagtgattttttataaatggtttatagaataaattgtacagcgtttaattggacttgctctctgaaataacgtaaaattgtagtgaggaggacggttacagatgaatcaaaaaaaaatatcattattcggagatct

SEQ ID NO: 16 - DNA, 219 bp, Artificial Sequence
  P8s with tails promoter clone sequence
agatctgcatccagtgaacctgccccctattctttttttaggacaattatttctcataaaaagcagattttagaaagaaaattgtatttttttaacagctttgactgccctttttggaagagtttatgtataatagaattagttagttttgctattgatatagcagcagaaatggagagatatgatgaatcaaaaaaaaatatcattattcggagatct

SEQ ID NO: 17 - DNA, 24 bp, Artificial Sequence
  PCR primer for leuA upstream
cgaggaccgagagacgtcctcacg

SEQ ID NO: 18 - DNA, 26 bp, Artificial Sequence
  PCR primer for leuA downstream
gcccaattccatctcccgcaagtgtc

SEQ ID NO: 19 - DNA, 20 bp, Artificial Sequence
  RT3-gadB-F forward primer for gadB
atgctccctttgttgagcca

SEQ ID NO: 20 - DNA, 20 bp, Artificial Sequence
  RT3-gadB-R reverse primer for gadB
acgccacaaaacccaaccta

SEQ ID NO: 21 - DNA, 20 bp, Artificial Sequence
  Ll16srRNA2-F forward primer for 16srRNA
acgagactgccggtgataaa

SEQ ID NO: 22 - DNA, 20 bp, Artificial Sequence
  Ll16srRNA2-R reverse primer for 16s rRNA
gagttgcagcctacaatccg

SEQ ID NO: 23 - DNA, 203 bp, Artificial Sequence
  synthesized P1 promoter
tctttaagttcataagtcatcaaaatttgctgacagactttcaatagagaacttctatttctgtcagtaattttttccactattttaaagaaattcacttcatcaattataccataaaccattttttaacagttttctttttacaaattgacaaaataagagtaaaattaaaatatagataaaaacttatgaaggagtcgcct

SEQ ID NO: 24 - DNA, 201 bp, Artificial Sequence
  synthesized P3 promoter
gcactgattcttttttcagtgcttttttttataaaaattggacaaaatagacatgttgtctaaaaactatttcagattcttgcaatcattttcttcttgtactatgatgaaattagtaagagattggaggagaattagaagtaaagagactaaaaaatttagtcaaactgatgttgtttagattagaggaggttaattatt

SEQ ID NO: 25 - DNA, 213 bp, Artificial Sequence
  synthesized P4 promoter
aacttcctaaagatttcaaaatcaaagttactttcttgttgttttaaattttacactttttgtaatgaactgtcaaactttaaaaagtagaaaatcaatttgcttgaattcacaattgcaaaaagatatactcaaattacaagagagaattacaaatacaacttaagtattcaattcataaagcaattgcacagttctattaaggaggaaaaa

SEQ ID NO: 26 - DNA, 203 bp, Artificial Sequence
  synthesized P6 promoter
agccatttttttgcaaaaaaagcatattagtaactaacaaaaatataaacaaaagtaaaataatagaatttaatgtacgaaatttattacaaatttgtattatattatgctataatatcctagaatcggtaatttttataagaaaatgtaatcaataaaatacatctcttgcaaaaatgatttaaaatcaaaaggaggacatt

SEQ ID NO: 27 - DNA, 351 bp, Artificial Sequence
  synthesized P7 promoter
agtgattattataaaagacaattctgataaagagttgttttttttaatttcttaagcaaatcttattcttgataagcaaactatttttgtgctataataaaaatatcttcagggcaccgtgtaattcgggaccggcggtaaatagggctttgaccttatgactccgcgattcgctacggcgattgaagcagtgagaatctgctagcgacagtaaagtctggatggaagaagatgaacaatttttgtagtgtaatcaacttcggttggattactgtttttagccaaaccaaaaatgtctatcaacttcctcgaaatttgaagaattattttctcatatttggaggttttttt